(19) United States
(12) Patent Application Publication
(10) Pub. No.: US 2005/0203079 A1
(43) Pub. Date: Sep. 15, 2005

(54) Compounds having affinity at 5ht2C receptor and use thereof in therapy

(75) Inventors: Federica Damiani, Verona (IT); Dieter Hamprecht, Verona (IT); Albert Andrzej Jaxa-Chamiec, Verona (IT); Fabrizio Micheli, Verona (IT); Alessandro Pasquarello, Verona (IT); Giovanna Tedesco, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED

(21) Appl. No.: 10/511,769

(22) PCT Filed: Apr. 17, 2003
(86) PCT No.: PCT/EP03/04180

§ 371 Date: May 2, 2005

(30) Foreign Application Priority Data
Apr. 19, 2002 (GB) 0209029.8
Sep. 6, 2002 (GB) 0220781.9

(51) Int. Cl. A61K 31/55; A61K 31/4439; A61K 31/4025; A61K 31/4015; C07D 43/02

(52) U.S. Cl. 514/212.03; 548/543; 546/290; 546/268.1; 540/524; 514/424; 514/422; 514/340; 514/345

(57) ABSTRACT

Compounds of formula (I) or a pharmaceutically acceptable salt thereof are disclosed:

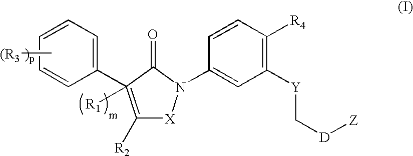

wherein R₁ is hydrogen, hydroxy, fluoro, chloro, C₁₋₆alkyl, C₃₋₇cycloalkyl, C₃₋₇cycloalkyloxy, C₁₋₆alkoxy or haloC₁₋₆alkoxy; m is 0 when 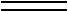
is a double bond and m is 1 when 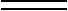
is a single bond; R₂ is hydrogen, halogen, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group; X is —(CH₂—CH₂)—, —(CH═CH)—, —(CH₂)₃—, —C(CH₃)₂—, —(CH═CH—CH₂)—, —(CH₂—CH═CH)— or a group —(CHR₅)— wherein R₅ is hydrogen, halogen, hydroxy, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy or C₁₋₆alkylthio; R₃ is halogen, cyano, C₁₋₆alkyl, C₃₋₇cycloalkyl, C₃₋₇cycloalkyloxy, C₁₋₆alkoxy, C₁₋₆alkylthio, hydroxy, amino, mono- or di-C₁₋₆alkylamino, an N-linked 4 to 7 membered heterocyclic group, nitro, haloC₁₋₆alkyl, haloC₁₋₆alkoxy, aryl, arylC₁₋₆alkyl, arylC₁₋₆alkyloxy, arylC₁₋₆alkylthio or COOR₆, CONR₇R₈ or COR₉ wherein R₆, R₇, R₈ and R₉ are independently hydrogen or C₁₋₆alkyl; p is 0, 1 or 2 or 3; R₄ is hydrogen, halogen, hydroxy, cyano, nitro, C₁₋₆alkyl, C₁₋₆alkanoyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group; Y is oxygen, sulfur, —CH₂— or NR₁₀ wherein R₁₀ is hydrogen or C₁₋₆alkyl; D is a single bond, —CH₂—, —(CH₂)₂— or —CH═CH—; and Z is an optionally substituted C-linked 4 to 7 membered heterocyclic group containing at least one nitrogen, an optionally substituted N-linked 4 to 7 membered heterocyclic group, or Z is —NR₁₁R₁₂ where R₁₁ and R₁₂ are independently hydrogen or C₁₋₆alkyl. Methods of preparation and uses thereof in therapy, particularly for CNS disorders such as depression and anxiety, are also disclosed.

This invention relates to novel compounds having pharmacological activity, processes for their preparation, to compositions containing them and to their use in the treatment of CNS and other disorders.

WO 96/23783, WO 97/46699 and WO 97/48700 all disclose a series of indoline derivatives which are 5-HT_{2C} receptor antagonists and which are claimed to be useful in the treatment of various CNS disorders.

A novel class of compounds possessing 5-HT_{2C} receptor activity has been found. The present invention therefore provides, in a first aspect, a compound of formula (I) or a pharmaceutically acceptable salt thereof: 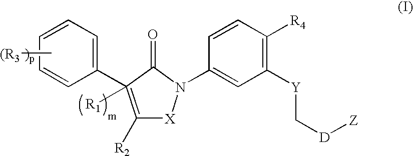
wherein:
R₁ is hydrogen, hydroxy, fluoro, chloro, C₁₋₆alkyl, C₃₋₇cycloalkyl, C₃₋₇cycloalkyloxy, C₁₋₆alkoxy or haloC₁₋₆alkoxy;
m is 0 when 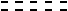
   is a double bond and m is 1 when 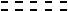
   is a single bond;
R₂ is hydrogen, halogen, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;
X is —(CH₂—CH₂)—, —(CH═CH)—, —(CH₂)₃—, —C(CH₃)₂—, —(CH═CH—CH₂)—, —(CH₂—CH═CH)— or a group —(CHR₅)— wherein R₅ is hydrogen, halogen, hydroxy, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy or C₁₋₆alkylthio;
R₃ is halogen, cyano, C₁₋₆alkyl, C₃₋₇cycloalkyl, C₃₋₇cycloalkyloxy, C₁₋₆alkoxy, C₁₋₆alkylthio, hydroxy, amino, mono- or di-C₁₋₆alkylamino, an N-linked 4 to 7 membered heterocyclic group, nitro, haloC₁₋₆alkyl, haloC₁₋₆alkoxy, aryl, arylC₁₋₆alkyl, arylC₁₋₆alkyloxy, arylC₁₋₆alkylthio or COOR₆, CONR₇R₈ or COR₉ wherein R₆, R₇; R₈ and R₉ are independently hydrogen or C₁₋₆alkyl;
p is 0,1 or 2or 3;
R₄ is hydrogen, halogen, hydroxy, cyano, nitro, C₁₋₆alkyl, C₁₋₆alkanoyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;
Y is oxygen, sulfur, —CH₂— or NR₁₀ wherein R₁₀ is hydrogen or C₁₋₆alkyl;
D is a single bond, —CH₂—, —(CH₂)₂— or —CH═CH—; and
Z is an optionally substituted C-linked 4 to 7 membered heterocyclic group containing at least one nitrogen, an optionally substituted N-linked 4 to 7 membered heterocyclic group, or Z is —NR₁₁R₁₂ where R₁₁ and R₁₂ are independently hydrogen or C₁₋₆alkyl.

The following terms, whether used alone or as part of another group are to be given the following meanings, unless otherwise stated.

The term “halogen” and its abbreviated form “halo” are used herein to describe fluorine, chlorine, bromine or iodine.

The term “alkyl” is used herein to describe a straight chain or branched fully saturated hydrocarbon group. “C₁₋₆alkyl” refers to alkyl groups having from one to six carbon atoms, including all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “C₁₋₆alkanoyl” refers to an alkanoyl group having from 1 to 6 carbon atoms, such as methanoyl (or “formyl”), ethanoyl (or “acetyl”), propanoyl, isopropanoyl, butanoyl, isobutanoyl, sec-butanoyl, pentanoyl, neopentanoyl, sec-pentanoyl, isopentanoyl, tertpentanoyl and hexanoyl.

The term “C₁₋₆alkoxy” refers to a straight chain or branched chain alkoxy (or “alkyloxy”) group having from one to six carbon atoms, including all isomeric forms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “C₃₋₇cycloalkyl” refers to a cycloalkyl group consisting of from 3 to 7 carbon atoms, such as cyclopropane, cyclobutane, cyclopentane, cyclohexane and cycloheptane. Optional substituents for C₃₋₇cycloalkyl includes one or more halogen, hydroxy, oxo, C₁₋₆alkyl, cyano, CF₃, OCF₃, C₁₋₆alkoxy and C₁₋₆alkanoyl.

The term “C₁₋₆alkylthio” refers to a straight chain or branched chain alkylthio group having from one to six carbon atoms, such as methylthio, ethylthio, propylthio, isopropylthio, butylthio, isobutylthio, sec-butylthio, tert-butylthio, pentylthio, neopentylthio, sec-pentylthio, n-pentylthio, isopentylthio, tert-pentylthio and hexylthio.

The term “mono- or di-C₁₋₆alkylamino” refers to an amino group which is substituted by one C₁₋₆alkyl group or an amino group which is substituted by two C₁₋₆alkyl groups, the two amino groups being the same or different. Examples of monoC1-6alkylamino include methylamine, ethylamine, propylamine, isopropylamine, butylamine, isobutylamine, sec-butylamine, tert-butylamine, pentylamine, neopentylamine, sec-pentylamine, n-pentylamine, isopentylamine, tert-pentylamine and hexylamine. Examples of di-C1-6alkylamino include dimethylamine, diethylamine, dipropylamine, diisopropylamine, dibutylamine, diisobutylamine, disec-butylamine, ditert-butylamine, dipentylamine, dineopentylamine, dihexylamine, butylmethylamino, isopropylmethylamino, ethylisopropylamino, ethylmethylamino, etc.

The term “aryl” is used herein to describe groups such as phenyl or naphthyl, which may be optionally substituted by one or more of C₁₋₆alkyl (to form “arylC₁₋₆alkyl”), halogen, CF₃ or C₁₋₆alkoxy (to form “arylC₁₋₆alkoxy”).

The terms “halo C₁₋₆alkoxy” or “haloC₁₋₆alkyl” are used to describe a C₁₋₆alkoxy or a C₁₋₆alkyl group, respectively, substituted with one or more halogens. Examples include —CHCl₂, —CF₃, —OCF₃, etc.

The term “heterocyclic group” is used herein to describe a stable aromatic or non-aromatic ring containing 1, 2 or 3 heteroatoms selected from nitrogen, sulphur and oxygen. Suitable examples of 4 to 7 membered heterocyclic groups include azetidinyl, pyrrolidinyl, imidazolidinyl, pyrazolidinyl, oxazolinyl, isothiazolidinyl, thiazolidinyl, pyrrolyl, pyrrolinyl, pyrazolinyl, imidazolyl, pyrazolyl, isothiazolyl, thiazolyl, piperidyl, piperazinyl, morpholinyl, pyridyl, pyridazinyl, pyrimidinyl, pyrazinyl, azepinyl, azepanyl, dioxolanyl, thienyl, tetrahydrothienyl, tetrahydrofuryl, dioxanyl and dithianyl.

The term “N-linked heterocyclic group” is used herein to describe a heterocyclic group which is linked to the remainder of the molecule via a nitrogen atom. Suitable examples of 4 to 7 membered N-linked heterocyclic groups include azetidinyl, pyrrolidinyl, imidazolidinyl, pyrazolidinyl, oxazolinyl, isothiazolidinyl, thiazolidinyl, pyrrolyl, pyrrolinyl, pyrazolinyl, imidazolyl, pyrazolyl, piperidyl, piperazinyl, morpholinyl and azepanyl.

The term “C-linked heterocyclic group containing at least one nitrogen” is used herein to describe a heterocyclic group which Is contains at least one nitrogen atom and is linked to the remainder of the molecule via a carbon atom. Suitable examples of 4 to 7 membered C-linked heterocyclic groups containing at least one nitrogen include azetidinyl, pyrrolidinyl, imidazolidinyl, pyrazolidinyl, pyrrolyl, pyrrolinyl, pyrazolinyl, imidazolyl, pyrazolyl, piperidyl, piperazinyl, morpholinyl, pyridyl, pyridazinyl, pyrimidinyl, pyrazinyl, azepinyl and azepanyl.

More than one optional substituent may be present in the N-linked or C-linked heterocyclic group, which may be the same or different, and may be attached to any carbon atom of the heterocyclic group or an available nitrogen atom.

Suitable optional substituents for the N-linked or C-linked heterocyclic group include C₁₋₆alkyl, amino, mono- or di-C₁₋₆alkylamino, aryl, arylC₁₋₆alkyl, arylamino, hydroxy, C₁₋₆alkylamido, hydroxyC₁₋₆alkyl, C₁₋₆alkoxycarbonyl, halogen, haloC1-6alkyl, a heteroaromatic group (such as indole or benzimidazole), an aromatic or non-aromatic N-linked or C-linked heterocyclic group or an aromatic or non-aromatic heterocycleC₁₋₆alkyl optionally substituted by C₁₋₆alkyl. Examples of aromatic or non-aromatic heterocycleC₁₋₆alkyl include heterocycle-methyl (such as pyridinyl-methyl and benzimidazolyl-methyl) and heterocycle-ethyl (such as morpholinyl-ethyl and indolyl-ethyl).

Substituents in the N-linked or C-linked heterocyclic group may form a bridge structure, to form a group such as for example 2-oxa-5-azabicyclo[2.2.1]heptyl. Such a bicyclic group may be further substituted by the substituents listed above. More than one substituent may be present on the same carbon atom to form spiro structures such as 1,4 and 1,5 dioxa Spiro compounds.

When X is a group —(CHR₅)—, preferably R₅ is hydrogen. Preferably X is —CH₂—.

When 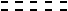
is a single bond, preferably R₁ is hydrogen, hydroxy or C₁₋₆alkoxy.

Preferably R₂ is hydrogen.

When p is 2 or 3, R₃ may be the same or different. Preferably p is 1 or 2 and R₃ is/are halogen, particularly chloro or fluoro, attached at the 3 or the 3,4-positions of the phenyl ring.

Preferably R₄ is C₁₋₆alkoxy (particularly methoxy), OCF₃, halogen or cyano.

Preferably Y is oxygen.

Preferably D is —CH₂—.

Preferably Z is an optionally substituted N-linked 4 to 7 membered heterocyclic group, in particular piperidyl. Preferred substituents include halogen (particularly fluoro) and C₁₋₆alkyl (particularly methyl).

Preferred compounds are compounds of formula (Ia): 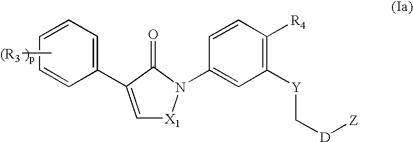
wherein R₃, p, R₄, Y, D, Z, 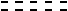
are as defined for formula (I) and X₁ is —CH₂— or —HC(OH)—. Preferred features of formula (I) also apply to formula (Ia).

Preferred compounds include:
1. 3-(3,4-Dichloro-phenyl)-3-hydroxy-1-[4-methoxy-3-(2-piperidin-1-yl-ethoxy)-phenyl)]-pyrrolidin-2-one
2. 3-(3,4-Dichloro-phenyl)-3-hydroxy-1-[4-methoxy-3-(2-morpholin-4-yl-ethoxy)-phenyl]-pyrrolidin-2-one
3. 3-(3,4-Dichloro-phenyl)-1-[4-methoxy-3-(2-piperidin-1-yl-ethoxy)-phenyl]-pyrrolidin-2-one
4. 1-[4-Chloro-3-(2-piperidin-1-yl-ethoxy)-phenyl]-3-(3,4-dichloro-phenyl)-pyrrolidin-2-one
5. 1-[4-Chloro-3-(2-piperidin-1-yl-ethoxy)-phenyl]-3-(3,4-dichloro-phenyl)-3-hydroxy-pyrrolidin-2-one
6. 3-(3,4-Dichloro-phenyl)-1-(4-methoxy-3-[2-(4methyl-piperidin-1-yl)-ethoxy]-phenyl)-pyrrolidin-2-one
7. 3-(3,4-Dichloro-phenyl)-1-[4-methoxy-3-(2-piperidin-1-yl-ethoxy)-phenyl]-1,5-dihydro-pyrrol-2-one
8. 3-(3,4-Dichloro-phenyl)-1-[4-methoxy-3-(2-morpholin-4-yl-ethoxy)-phenyl]-1,5-dihydro-pyrrol-2-one
9. 1-[4-Chloro-3-(2-piperidin-1-yl-ethoxy)-phenyl]-3-(3,4-dichloro-phenyl)-1,5-dihydro-pyrrol-2-one
10. 3-(3,4-Dichloro-phenyl)-1-[4-methoxy-3-(2-pyrrolidin-1-yl-ethoxy)-phenyl]-1,5-dihydro-pyrrol-2-one
11. 3-(3-Fluoro-phenyl)-1-[4-methoxy-3-(2-piperidin-1-yl-ethoxy)-phenyl]-1,5-dihydro-pyrrol-2-one
12. 3-(3,4-Dichloro-phenyl)-1-(-3-[2-(4,4-difluoro-piperidin-1-yl)-ethoxy]-4-methoxy-phenyl)-1,5-dihydro-pyrrol-2-one
13. 3-(3-Fluoro-phenyl)-5-hydroxy-1-[4-methoxy-3-(2-piperidin-1-yl-ethoxy)-phenyl]-pyrrolidin-2-one
14. 1-[4-Chloro-3-(2-piperidin-1-yl-ethoxy)-phenyl]-3-(3,4-dichloro-phenyl)-5-hydroxy-pyrrolidin-2-one
15. 3-(3,4-Dichloro-phenyl)-1-(-3-[2-(4,4-difluoro-piperidin-1-yl)ethoxy]-4-methoxy-phenyl)-5-hydroxy-pyrrolidin-2-one
16. 3-(3-Fluoro-phenyl)-1-[4-methoxy-3-(2-piperidin-1-yl-ethoxy)-phenyl]-pyrrolidin-2-one
17. 3-(3,4-Dichloro-phenyl)-1-[4-methoxy-3-(2-pyrrolidin-1-yl-ethoxy)-phenyl]-pyrrolidin-2-one
18. 3-(3,4-Dichloro-phenyl)-1-[4-methoxy-3-(2-piperidin-1-yl-ethoxy)-phenyl]-3-methyl-pyrrolidin-2-one
19. 3-(3-Chloro-phenyl)-1-{4-methoxy-3-[2-(4-methyl-piperidin-1-yl)-ethoxy]-phenyl}-1,5-dihydro-pyrrol-2-one
20. 3-(3,4-Dichloro-phenyl)-1-[4methoxy-3-(2-piperidin-1-yl-ethoxy)-phenyl]-3,4-dihydro-1H-pyridin-2-one
21. 3-(3,4-Dichloro-phenyl)-1-[4methoxy-3-(2-piperidin-1-yl-ethoxy)-phenyl]-piperidin-2-one
22. 3-(3,4-Dichloro-phenyl)-1-(4-methoxy-3-[2-(4-methyl-piperidin-1-yl)-ethoxy]-phenyl)-3,4-dihydro-pyrrol-2-one
23. 3-(3,4Dichloro-phenyl)-1-(4-methoxy-3-[2-(4-methyl-piperidin-1-yl)-ethoxy]-phenyl)-4-methyl-1,5-dihydro-pyrrol-2-one
24. 3-(4-Chloro-phenyl)-1-(4-methoxy-3-[2-(4-methyl-piperidin-1-yl)-ethoxy]-phenyl)-3,4-dihydro-pyrrol-2-one
25. 3-(4Chloro-phenyl)-1-(4-methoxy-3-[2-piperidin-1-yl)-ethoxy]-phenyl)-3,4-dihydro-pyrrol-2-one
26. 3-(3,4-Dichloro-phenyl)-1-[4-methoxy-3-(piperidin-3-ylmethoxy)-phenyl]-pyrrolidin-one
27. 3-(3,4-Dichloro-phenyl)-1-[4-methoxy-3-(1-methyl-piperidin-3-ylmethoxy)-phenyl]-pyrrolidin-one
28. N-{4-[3-(3,4-dichlorophenyl)-2-oxo-2,5-dihydro-1H-pyrrol-1-yl]-2-[2-(1-piperidinyl)ethoxy]phenyl}acetamide
29. N-{4-[3-(3,4dichlorophenyl)-2-oxo-pyrrolidin-1-yl]-2-[2-(1-piperidinyl)ethoxy]phenyl}acetamide
30. 3-(3,4-Dichloro-phenyl)-1-{4-methoxy-3-[2-(4methyl)-piperidin-1-yl-ethoxy]-phenyl}-3-methyl-pyrrolidin-2-one
31. 3-(3-Chloro-phenyl)-1-[4-methoxy-3-(2-piperidin-1-yl-ethoxy)phenyl]-pyrrolidin-2-one
32. 3-(3-Trifluoromethyl-phenyl)-1-[4-methoxy-3-(2-piperidin-1-yl-ethoxy)phenyl]-pyrrolidin-2-one
33. 3-(3-Trifluoromethyl-phenyl)-1-[4-methoxy-3-(2-piperidin-1-yl-ethoxy)phenyl]-1,5 dihydro-pyrrol-2-one
34. 3-(3-Chloro-phenyl)-5-methoxy-1-[4-methoxy-3-(2-piperidin-1-yl-ethoxy)-phenyl]-pyrrolidin-2-one
35. 3-(3-Chloro-phenyl)-1-[4-methoxy-3-(2-piperidin-1-yl-ethoxy)-phenyl]-1,5-dihydro-pyrrol-2-one
36. 3-(4Fluoro-phenyl)-1-[4-methoxy-3-(2-piperidin-1-yl-ethoxy)-phenyl]-1,5-dihydro-pyrrol-2-one
37. 3-(4Fluoro-phenyl)-1-[4-methoxy-3-(2-piperidin-1-yl-ethoxy)-phenyl]-pyrrolidin-2-one
38. 1-{4-Methoxy-3-[2-(4methyl-piperidin-1-yl)ethoxy]-phenyl}-3-(4methyl-phenyl)-1,5-dihydro-pyrrol-2-one
39. 1-{4-Methoxy-3-[2-(piperidin-1-yl)-ethoxy]-phenyl}-3-(4-methyl-phenyl)-1,5-dihydro-pyrrol-2-one
40. 3-(4-Bromo-phenyl)-1-{4-methoxy-3-[2-(4-methyl-piperidin-1-yl)-ethoxy]-phenyl}-1,5-dihydro-pyrrol-2-one
41. 1-{4-Methoxy-3-[2-(4-methyl-piperidin-1-yl)-ethoxy]-phenyl}-3-(4-trifluoromethyl-phenyl)-1,5-dihydro-pyrrol-2-one
42. 3-(2-Chloro-phenyl)-1-{4-methoxy-3-[2-(4methyl-piperidin-1-yl)-ethoxy]-phenyl}-1,5-dihydro-pyrrol-2-one
43. 3-(3,4-Dichloro-phenyl)-3-hydroxy-1-{4-methoxy-3-[2-(4-methyl-piperidin-1-yl)-ethoxy]-phenyl}-pyrrolidin-2-one
44. 3-(3,4-Dichloro-phenyl)-3-fluoro-1-{4methoxy-3-[2-(4methyl-piperidin-1-yl)-ethoxy]-phenyl}-pyrrolidin-2-one
45. 3-(3,4Dichloro-phenyl)-1-{4-methoxy-3-[(1-methyl-pyrrolidin-2-yl)-methoxy]-phenyl}-pyrrolidin-2-one
46. 3-(3,4-Dichloro-phenyl)-1-{4-methoxy-3-[(1-methyl-pyrrolidin-2-yl)-methoxy]-phenyl}-3,4-dihydro-pyrrol-2-one
   and pharmaceutically acceptable salts thereof.

The compounds of formula (I) can form acid addition salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid.

The compounds of this invention may be in crystalline or non-crystalline form, and, if crystalline, may optionally be hydrated or solvated. This invention includes within its scope stoichiometric hydrates as well as compounds containing variable amounts of water.

Certain compounds of formula (I) are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises:
(a) reacting a compound of formula (II): 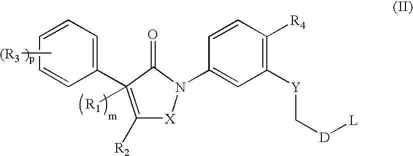
   wherein R₁, R₂, R₃, R₄, m, p, X, 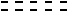
   Y and D are as defined for formula (I), and L is a leaving group, with a compound of formula (Ill):
   Z-H (III)
   wherein Z is as defined for formula (I); or
(b) cyclising a compound of formula (IV): 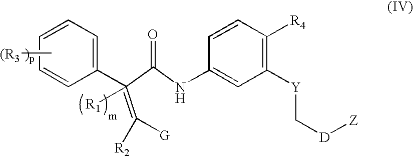
   wherein R₁, R₂, m, R₃, p, R₄, Y, D, Z and 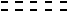
   are as defined for formula (I) and G is a group —X═CH₂, wherein X is as defined for formula (I), dehydrogenated as required;
optionally followed by:
   removing any protecting groups; and/or
   converting a compound of formula (I) into another compound of formula (I); and/or
   forming a pharmaceutically acceptable salt.

For the reaction of process (a), suitably L is mesylate. The reaction may take place in a solvent such as DMF In the presence of sodium iodide and potassium carbonate.

The reaction of process (b) suitably takes place in a solvent such as THF in the presence of OsO₄ and NalO₄.

Compounds of formula (I) can be converted into further compounds of formula (I) using standard techniques. For example, and by way of illustration rather than limitation, a compound wherein X is —(HCOH)— may be converted to a compound wherein X is —(CH₂)— by using a suitable reducing agent such as triethylsilane-trifluoroacetic acid using dichloromethane as solvent, and a compound wherein R₁ is hydroxy may be converted to compound wherein m is 0 and 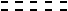
is a double bond by an elimination reaction in TFA.

Compounds of formulae (II), (III) and (IV) are commercially available or may be prepared according to methods described herein or may be prepared according to known methods or by analogous methods thereto.

Those skilled in the art will appreciate that it may be necessary to protect certain groups to carry out the above processes. Suitable protecting groups and methods for their attachment and removal are conventional in the art of organic chemistry, such as those described in Greene T. W. ‘Protective groups in organic synthesis’ New York, Wiley (1981).

Pharmaceutically acceptable salts may be prepared conventionally by reaction with the appropriate acid or acid derivative.

In another aspect, the present invention provides a pharmaceutical composition comprising a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol) syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented In unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The compounds of the present invention have affinity for the 5-HT_{2C} receptor. The affinity can be determined by assessing their ability to displace [³H]-mesulergine from rat or human 5-HT_{2C} clones expressed In 293 cells in vitro, as described in WO 94/04533.

All the Example compounds were tested according to this assay and were found to have pKi values >5.8. Some compounds show a considerably higher affinity in the range of 7.0 to >9.0 in human cells.

The intrinsic activity of the compounds of this invention can be determined according to the [³⁵S]GTP_{γ}S functional assay which is described in WO 99/07700.

Compounds of formula (I) and their pharmaceutically acceptable salts are of use in the treatment of certain CNS disorders such as depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, vascular dementia with depressed mood, schizoaffective disorder or the depressed type, and depressive disorders resulting from general medical conditions including, but not limited to, myocardial infarction, diabetes, miscarriage or abortion, etc), anxiety including generalised anxiety and social anxiety disorder, schizophrenia, panic disorder, agoraphobia, social phobia, epilepsy, obsessive compulsive disorder and post-traumatic stress disorder, pain (particularly neuropathic pain), migraine, memory disorders, including dementia, amnesic disorders and age-associated memory impairment, disorders of eating behaviours including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof, Alzheimer's disease, motor disorders such as Parkinson's disease, dementia in Parkinson's disease, neuroleptic-induced Parkinsonism and tardive dyskinesias, as well as other psychiatric disorders, disorders associated with spinal trauma and/or head injury such as hydrocephalus, gastrointestinal disorders such as IBS (Irritable Bowel Syndrome), Crohn's disease, ulcerative colitis, non-steroidal anti-inflammatory drug induced damage) as well as microvascular diseases such as macular oedema and retinopathy.

It is to be understood that, as used herein, the term “treatment” refers to alleviation of established symptoms as well as prophylaxis.

Thus the present invention also provides a compound of formula (I) or a pharmaceutically acceptable salt thereof, for use as a therapeutic substance. In particular, the present invention provides a compound of formula (I) or a pharmaceutically acceptable salt thereof for use in the treatment of the above disorders. In particular the invention provides a compound of formula (I) or a pharmaceutically acceptable salt thereof for use as a therapeutic substance In the treatment of a CNS disorder. Preferably the CNS disorder is depression or anxiety.

Compounds of the invention may be administered in combination with other active substances such as 5HT3 antagonists, NK-1 antagonists, serotonin agonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

The invention further provides a method of treatment of the above disorders in mammals including humans, which comprises administering to the sufferer a therapeutically safe and effective amount of a compound of formula (I) or a pharmaceutically acceptable salt thereof. In particular the invention provides a a method of treatment of a CNS disorder in mammals including humans, which comprises administering to the sufferer a therapeutically safe and effective amount of a compound of formula (I) or a pharmaceutically acceptable salt thereof. Preferably the disorder is depression or anxiety.

In another aspect, the invention provides for the use of a compound of formula (I) or a pharmaceutically acceptable salt thereof in the manufacture of a medicament for use in the treatment of the above disorders. In particular the present invention provides the use of a compound of formula (I) or a pharmaceutically acceptable salt thereof in the manufacture of a medicament for use in the treatment of a CNS disorder. Preferably the CNS disorder is depression or anxiety.

The composition of the present invention may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months. When administered in accordance with the invention, no unacceptable toxicological effects are expected with the compounds of the invention.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the present invention.
Preparation 1: 1-Benzyl-4,4-difluoro-piperidine

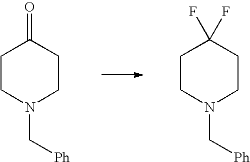

Procedure:

Chem. Pharm. Bull. 1993, 41, 11, 1971.

NMR (¹H, CDCl₃): δ 7.4-7.25 (m, 5H), 3.55 (s, 2H), 2.54 (m, 4H), 1.99 (m, 4H). MS (m/z):212 [MH]⁺
Preparation 2: 4,4-Difluoro-piperidine Hydrochloride

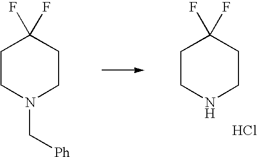

Procedure:

Chem. Pharm. Bull. 1993, 41, 11, 1971.

NMR (¹H, DMSO-d6): δ 9.22 (bs, 2H), 3.20 (m, 4H), 2.25 (m, 4H). MS (m/z): 122 [MH-HCl]⁺
Preparation 3: 2-Chloro-1-(4,4-difluoro-piperidin-1-yl)-ethanone

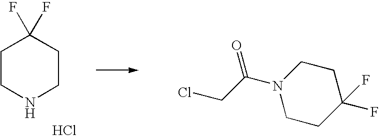

Procedure:

To a solution of 4,4-difluoro-piperidine hydrochloride (1120 mg, 7.13 mmol) in anh. CH₂Cl₂ (10 ml) was added, at 0° C. and. under N₂, TEA (2.5 eq, 2.18 ml) and chloroacetyl chloride (1.1 eq, 661 μl). The reaction was stirred at room temperature for 1 hour. The solution was diluted with water (30 ml) and extracted with ethyl acetate (3×25 ml). The combined organic extracts were dried over anhydrous Na₂SO₄, filtered and concentrated to dryness in vacuo to give 1128 mg of the title product (yield: 80.3%).

NMR (¹H, DMSO-d6): δ 4.44 (s, 2H), 3.55 (m, 4H), 2.0 (m, 4H). MS (m/z): 198 [MH]⁺, 1Cl
Preparation 4: 2-Chloro-5-nitro-phenol

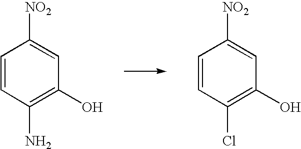

Procedure:

J. Chem. Soc. 1896, 69,1326.

NMR (¹H, DMSO-d6): δ 11.3 (s, 1H), 7.75 (d, 1H), 7.66 (d, 1H), 7.64 (s, 1H).
Preparation 5: Acetic Acid 2-(2-methoxy-5-nitro-phenoxy)-ethyl Ester

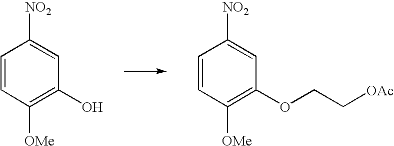

Procedure:

To a solution of 2-methoxy-5-nitro-phenol (2202 mg, 13.02 mmol) in acetone (110 ml), under N₂, was added K₂CO₃ (4.45 eq., 8000 mg) and 2-bromoethyl acetate (2.1 eq, 3 ml). The mixture was heated to reflux for 20 hours, filtered and then concentrated in vacuo. The crude product was triturated with AcOEt/cHex 1/1 (70 ml) to give 2999 mg of the title product as a white solid (90.2%, mp: 120-121.3° C.).

NMR (¹H, CDCl₃): δ 7.9 (dd, 1H), 7.75 (d, 1H), 6.9 (d, 1H), 4.45 (m, 2H), 4.3 (m, 2H), 3.95 (s, 3H), 2.05 (s, 3H).
Preparation 6: 1-[2-(2-Methoxy-5-nitro-phenoxy)-ethyl]-piperidine

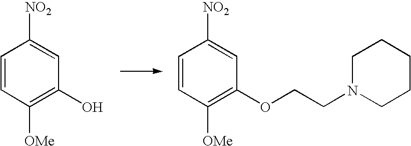

Procedure:

To a solution of 2-methoxy-5-nitrophenol (1.5 g, 8.87 mmol) in DMF (18 ml) at room temperature were added K₂CO₃ (2.70 g, 19.52 mmol) and (1-(2-chloroethyl)piperidin hydrochloride) (1.79 g, 9.75 mmol). The suspension was stirred at room temperature for 24 hours. The suspension was diluted with water (20 ml) and extract with EtOAc (2×20 ml), the combined organic phases were dried over Na₂SO₄, filtered and concentrated to dryness in vacuo, to obtain the title product as a yellow oil.

NMR (¹H, CDCl₃): δ 7.85 (dd, 1H), 7.75 (d, 1H), 6.90 (d, 1H), 4.20 (t, 2H), 3.90 (s, 3H), 2.80 (t, 2H), 2.45-2.60 (m, 4H), 1.55-1.65 (m, 4H) (m, 4H), 1.35-1.50 (m, 2H).

MS (m/z): 281[MH]⁺.
Preparation 7: 1-(4,4-Difluoro-piperidine-1-yl)-2-(2-methoxy-4-nitro-phenoxy)-ethanone

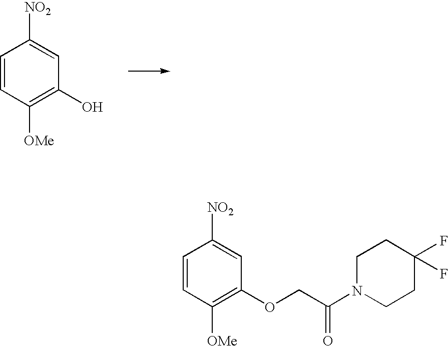

Procedure:

2-Methoxy-nitro-phenol (177 mg, 1.05 mmol), was dissolved in dry DMF (2 ml) under N₂. Potassium carbonate (158 mg, 1.1 eq) was added and the mixture was stirred for 15 min, then 2-chloro-1-(4,4-difluoro-piperidin-1-yl)-ethanone (228 mg, 1.1 eq) dissolved in dry DMF (3 ml) was added. After 20 hours a saturated solution of NH₄Cl was added and the mixture extracted with CH₂Cl₂ (2×25 ml). The organic phase was washed with NaOH 0.1 N (50 ml), water (50 ml), dried over Na₂SO₄ and concentrated in vacuo to give 405 mg of crude title product (mp 127-130° C.) that was utilized without further purification.

NMR (¹H, CDCl₃): δ 7.98 (dd, 1H), 7.77 (d, 1H), 6.96 (d, 1H), 4.84 (s, 2H), 3.98 (s, 3H), 3.8-3.65 (m, 4H), 2.15-1.95 (m, 4H). MS (m/z): 331 [MH]⁺.
Preparation 8: 1-[2-(2-Chloro-5-nitro-phenoxy)-ethyl]-piperidine

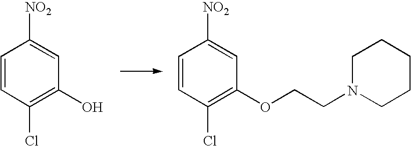

NMR (¹H, CDCl₃): δ 7.84 (d, 1H), 7.81 (dd, 1H), 7.52 (d, 1H), 4.30 (t, 2H), 2.92 (t, 2H), 2.6 (m, 4H), 1.5-1.7 (m, 6H). MS (m/z): 285 [MH]⁺, 1Cl.
Preparation 9: Acetic Acid 2-(5-amino-2-methoxy-phenoxy)-ethyl Ester

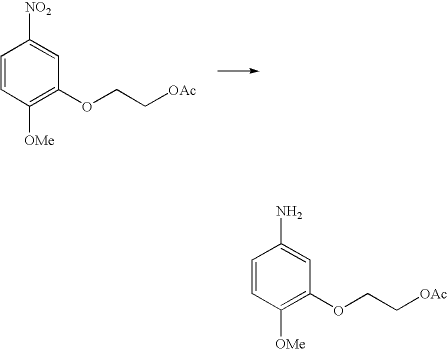

Procedure:

To a suspension of acetic acid 2-(2-methoxy-5-nitro-phenoxy)-ethyl ester (2904 mg, 11.38 mmol) in methanol (85 ml) was added, under N₂, ammonium formate (5.4 eq, 3900 mg) and Pd/C 10% (cat, 1100 mg). The reaction was stirred at room temperature for 1.5 hours, then filtered on celite pad and concentrated to dryness in vacuo. The crude product was then dissolved in CH₂Cl₂ (50 ml) and the organic phase was washed with brine (2×30 ml) and water (1×30 ml). The combined organic extracts were dried over anhydrous Na₂SO₄ filtered and concentrated to dryness in vacuo. Flash chromatography of the crude product (silica gel, cHex/AcOEt 4/6) gave 2299 mg of the title product as a white foam (yield: 89.7%).

NMR (¹H, CDCl₃): δ 6.7 (dd, 1H), 6.3 (d, 1H), 6.25 (dd, 1H), 4.4 (m, 2H), 4.15 (m, 2H), 3.75 (s, 3H), 2.05 (s, 3H). MS (m/z): 226.3 [MH]⁺.
Preparation 10: 4-Methoxy-3-(2-piperidin-1-yl)-ethoxy-phenylamine Hydrochloride

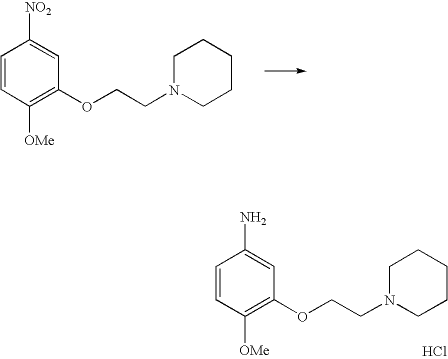

Procedure:

To a solution of the compound of Preparation 6 (2.30 g, 8.21 mmol) in MeOH (43.18 ml) were added a solution of NH₄Cl (5 eq, 2.20 g) in H₂O (36.83 ml) and iron (3 eq, 1.38 g). After stirring at reflux for 3 hours, the solution was concentrated in vacuo and the crude was tritured with CH₂Cl₂ to obtain 1.82 g of th title compound as a brown gum (yield: 76%).

(¹H, DMSO-d6): δ 10.4-10.0 (broad, 1H), 6.72 (d, 1H), 6.34(d, 1H), 6.20 (dd, 1H), 5.4-5.0 (broad, 2H), 4.24 (t, 2H), 3.64 (s, 3H), 3.52 (bs, 2H), 3.42 (t, 2H), 3.00 (bs, 2H), 1.9-1.3 (bs, 6H). MS (m/z): 251[MH]⁺.
Preparation 11: 4-Chloro-3-(2-piperidin-1-yl-ethoxy)-phenylamine

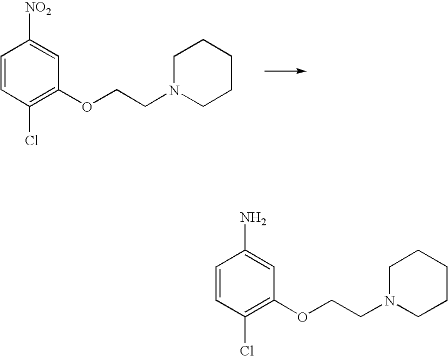

Procedure:

To a solution of 1-[2-(2-chloro-5-nitro-phenoxy)ethyl]-piperidine (782 mg, 2.75 mmol) in MeOH (8 ml) were added a solution of NH₄Cl (5 eq, 735 mg) in H₂O (8 ml) and iron (3 eq, 457 mg). After stirring at reflux for 12 hours, the solution was concentrated in vacuo and chromatographated over silica gel (CH₂Cl₂/MeOH 85/15) to give 757 mg of the title product as a green foam (yield: 87%).

NMR (¹H, CD₃OD): δ 7.0 (d, 1H), 6.4 (d, 1H), 6.2 (dd, 1H), 4.25 (t, 2H), 3.35 (t, 2H), 3.25 (s, 2H), 3.15 (m, 4H), 1.8-1.5 (m, 6H). MS (m/z): 255 [MH]⁺, 1Cl.
Preparation 12: 2-(3,4-Dichloro-phenyl)-pent-4-enoic Acid

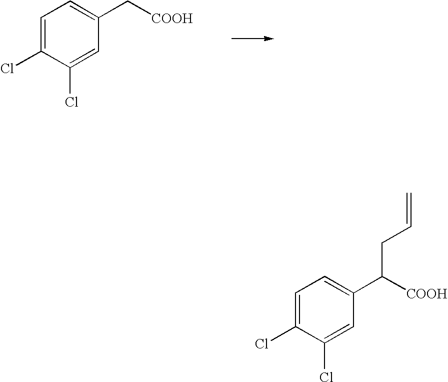

Procedure:

A solution of 3,4dichloro-phenylacetic acid (1.47 g, 7.17 mmol) in anh. THF (21 ml), under N₂, was treated with lithium bis(trimethylsilyl) amide (1M solution in THF, 2.2 eq., 16 ml) at −78° C. for 30 minutes before allyliodide(1.65 eq., 1.1 ml) was added. The mixture was stirred for 3 hours at room temperature and quenched with water (20 ml). The reaction mixture was acidified to pH=4 with HCl 1N. The product was extracted with ethyl acetate (2×15 ml) and the organic phase was washed with brine (1×15 ml), dried over Na₂SO₄ and concentrated in vacuo. The crude product was purified by flash chromatography (silica gel, AcOEt/cHex 2/8) to give 1137 mg of the title product as a pale yellow solid (81%).

NMR (¹H, CDCl₃): δ 7.4 (m, 2H), 7.15 (dd, 1H), 5.7 (m, 1H), 5.05 (m, 2H), 3.6 (t, 1H), 2.8 (m, 1H), 2.5 (m, 1H). MS (m/z): 244 [MH]⁺ +2Cl.
Preparation 13: 2-(3-Fluoro-phenyl)-pent-4-enoic Acid

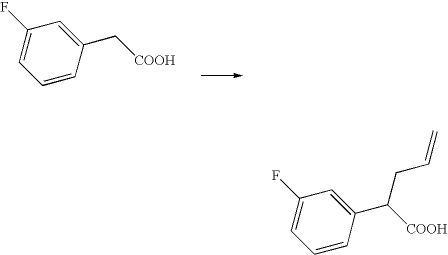

Procedure:

To a solution of 3-fluorophenylacetic acid (1.0 g, 6.49 mmol) in THF (10 mL), at −78° C. was added lithium bis(trimethylsilyl)amide (14.28 mmol). To solution was stirred at this temperature for 30 min and allyl bromide (0.84 mL. 9.74 mmol) was added. The solution was stirred at room temperature for 3 hours. Then the solution ws diluted with water, acidified with HCl 2 N and extracted with EtOAC (3×). The combined organic phases were dried over anhydrous Na₂SO₄ filtered and concentrated to dryness in vacuo. Flash chromatography of the crude product (silica gel, cHex/AcOEt 7/3) gave 1.120 g of the title product (yield=89%, mp=49° C.)

NMR (¹H, DMSO-d6): δ 12.50 (bs, 1H), 7.35 (m, 1 H), 7.00-7.25 (m, 3H), 5.60-5.80 (m, 1H), 4.90-5.10 (m, 1H), 3.65 (t, 1H), 2.60-2.70 (m, 1H), 2.35-2.50 (m, 1H).
Preparation 14: Acetic Acid 2-(5-[2-(3,4-dichloro-phenyl)-pent-4-enoylamine]-2-methoxy-phenoxy)-ethyl Ester

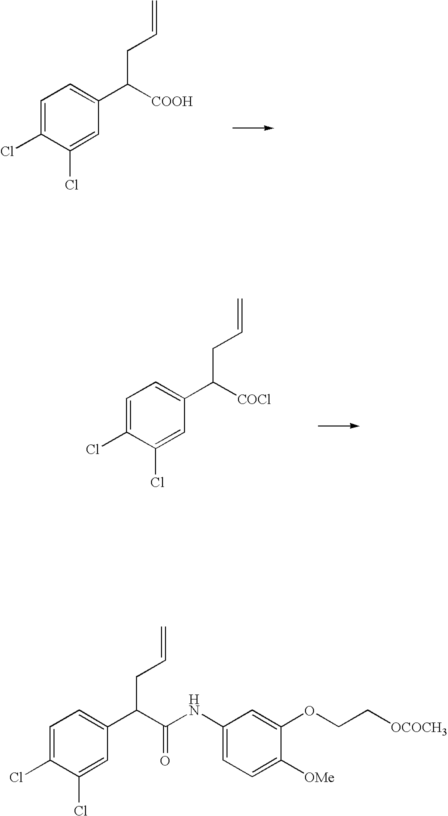

Procedure:

To a solution of 2-(3,4-dichloro-phenyl)-pent-4-enoic acid (222.4 mg, 0.907 mmol) in anh. CH₂Cl₂ (4 ml), under N₂, was added, at 0° C., oxalyl chloride (1.9 eq., 0.15 ml) and DMF (cat). The reaction was stirred at 0° C. for 15′ and at room temperature for 18 hours. The reaction mixture was concentrated to dryness in vacuo and the crude intermediate was then dissolved, under N₂, in anh. CH₂Cl₂ (5 ml). Acetic acid 2-(5-amino-2-methoxy-phenoxy)-ethyl ester (VVSI/6002/42/1) (1.22 eq, 250 mg) and TEA (2 eq., 0.25 ml) were added to the reaction mixture at 0° C. The reaction was stirred at 0° C. for 15′ and at room temperature for 18 hours. The reaction mixture was concentrated to dryness in vacuo The crude intermediate was then dissolved in CH₂Cl₂ and the organic phase was washed with NH₄Cl sat (2×10 ml) and brine (2×10 ml). Flash chromatography of the crude product (silica gel, cHex/AcOEt 7/3) gave 259 mg of the title product as a white (yield: 63.1%, mp: 112.7-114.2)

NMR (¹H, DMSO-d6): δ 10.02 (s, 1H), 7.60 (m, 2H), 7.36 (dd, 1H), 7.29 (d, 1H), 7.07 (dd, 1H), 6.88 (d, 1H), 5.70 (m, 1H), 5.07 (m, 1H), 4.98 (m, 1H), 4.30 (t, 2H), 4.09 (t, 2H), 3.74 (m, 1H), 3.70 (s, 3H), 2.75 (m, 1H), 2.45 (m, 1H), 2.02 (s, 3H) MS (m/z): 452 [MH]⁺+2Cl mp: 112.7-114° C.
Preparation 15: Acetic Acid 2-(5-[2-(3-fluoro-phenyl)-pent-4-enoylamino]-2-methoxy-phenoxy)-ethyl Ester

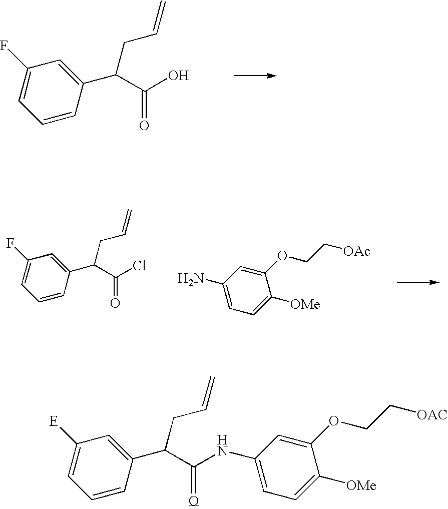

NMR (¹H, CDCl₃): δ 7.36 (d, 1H), 7.34 (m, 1H), 7.15 (d, 1H), 7.10 (dd, 1H), 7.04 (bs, 1H), 7.00 (m, 1H), 6.77-6.84 (m, 1H), 5.74 (m, 1H), 5.00-5.14 (m, 2H), 4.42 (t, 2H), 4.22 (t, 2H), 3.82 (s, 3H), 3.51 (t, 1H), 2.96 (m, 1H), 2.58 (m, 1H), 2.08 (s, 3H). MS (m/z):402 [MH]⁺. mp: 108.7-109.3° C.
Preparation 16: 2-(3,4-Dichloro-phenyl)-pent-4-enoic-acid [4-chloro-3-(2-piperidin-1-yl-ethoxy)-phenyl]-amide

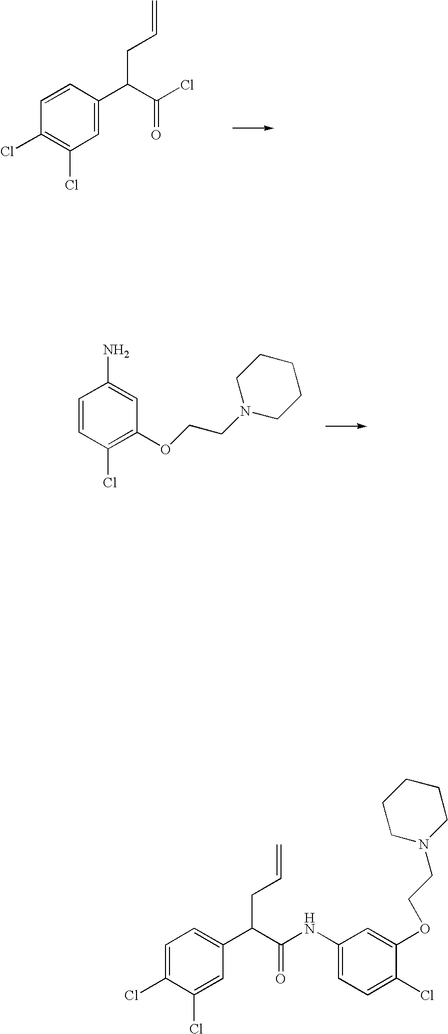

NMR (¹H, CD₃OD): δ 7.58 (d, 1H), 7.54 (d, 1H), 7.48 (d, 1H), 7.33 (dd, 1H), 7.27 (d, 1H), 7.0 (dd, 1H), 5.77 (m, 1H), 5.15-5.0 (m, 2H), 4.23 (t, 2H), 3.70 (t, 1H), 3.02 (t, 2H), 2.9 (m, 1H), 2.82 (m, 4H), 2.5 (m, 1H), 1.69 (m, 4H), 1.54 (m, 2H). MS (m/z): 481 [MH] +3Cl.
Preparation 17: 2-(3,4-Dichloro-phenyl)-pent-4-enoic Acid (3-[2-(4,4-difluoro-piperidin-1-yl)-ethoxy]-4-methoxy-phenyl)-amide

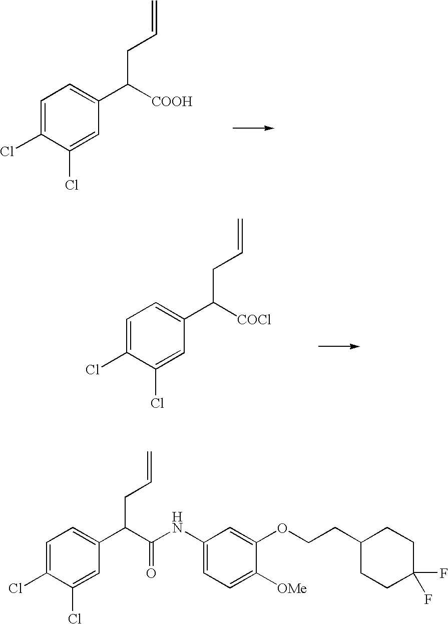

NMR (¹H, DMSO-d6): δ 9.96 (s, 1H), 7.57 (d, 1H), 7.55 (d, 1H), 7.32 (dd, 1H), 7.25 (d, 1H), 7.01 (dd, 1H), 6.82 (d, 1H), 5.61 (m, 1H), 5.06-4.93 (m, 2H), 3.96 (m, 2H), 3.72 (t, 1H), 3.65 (s, 3H), 2.72 (t, 2H), 2.56 (t, 4H), 2.45 (m, 2H), 1.9 (m, 4H) MS (m/z): 513[MH]⁺+2Cl.
Preparation 18: Acetic Acid 2-(5-[3-(3,4-dichloro-phenyl)-5-hydroxy-2oxo-pyrrolidine-1-yl]-2-methoxy-phenoxy)-ethyl Ester

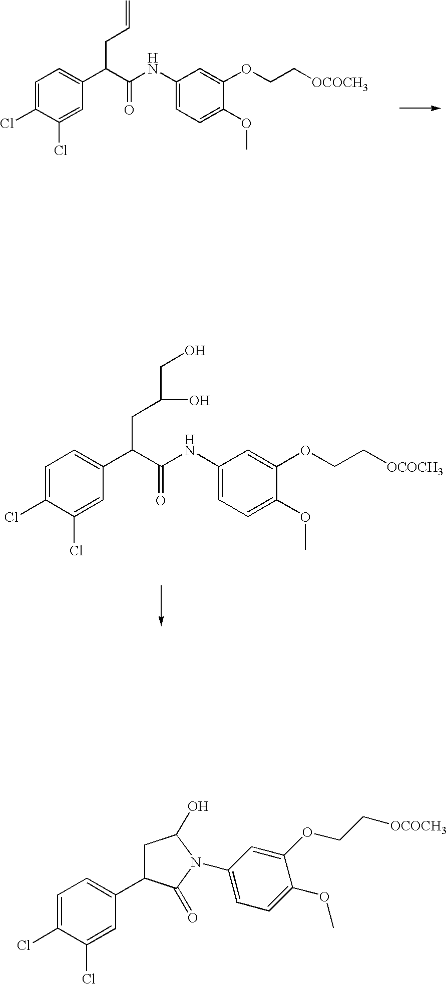

Procedure:

To a solution acetic acid 2-(5-[2-(3,4-dichloro-phenyl)-pent-4-enoylamine]-2-methoxy-phenoxy)-ethyl ester (950 mg, 2.1 mmol) in acetone/H₂O 8/1 (37.5/4.6 ml) was added N-methyl-morpholine-N-oxide (2 eq, 493 mg) and OsO₄ 4 wt % sol. in water (cat, 1 ml). The reaction was stirred at room temperature for 18 hours and then quenched with 40 ml of Na₂SO₃ sat. After 15 minutes stirring the diol was extracted with ethyl acetate (2×20 ml), dried over Na₂SO₄, filtered and concentrated to dryness in vacuo. The crude product was then dissolved in THF/H₂O 1/1 (20/20 ml) and potassium periodate (1.5 eq, 673.7 mg) was added. The reaction was stirred at room temperature for 2 hours. The solution was diluted with water (10 ml) and extracted with ethyl acetate (3×10 ml). The combined organic extracts were dried over anhydrous Na₂SO₄ filtered and concentrated to dryness in vacuo. Flash chromatography of the crude product (silica gel, cHex/AcOEt 5/15) gave 170 mg of one diastereoisomer of the title product, 227 mg of the other diastereoisomer, and 236 mg of a mixture of both as a white foam (yield: 66% in 2 steps)

(51-1)

NMR (¹H, DMSO-d6): δ 7.61 (d, 1H), 7.60 (d, 1H), 7.32 (dd, 1H), 7.26 (d, 1H), 7.11 (dd, 1H), 6.98 (d, 1H), 6.44 (d, 1H), 5.60 (t, 1H), 4.31 (t, 2H), 4.18 (t, 2H), 4.13 (t, 1H), 3.75 (s, 3H), 2.5-2.3 (m, 2H), 2.03 (s, 3H).

MS (m/z): 454 [MH] +2Cl.

(51-3)

NMR (¹H, DMSO-d6): δ 7.68 (d, 1H), 7.63 (d, 1H), 7.41 (dd, 1H), 7.12 (d, 1H), 6.89 (m, 2H), 6.52 (d, 1H), 5.66 (m, 1H), 4.31 (m, 2H), 4.13 (m, 2H), 3.87 (dd, 1H), 3.75 (s, 3H), 2.9 (m, 2H), 2.02 (s, 3H), 1.9 (m, 1H).
Preparation 19: Acetic Acid 2-(5-[3-(3,4-dichloro-phenyl)-2-oxo-pyrrolidine-1-yl]-2-methoxy-phenoxy)-ethyl Ester

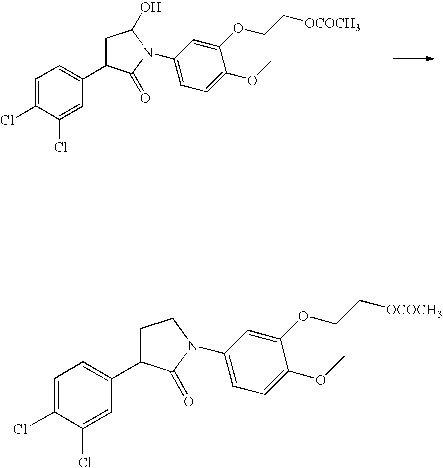

Procedure:

To a solution of acetic acid 2-(5-[3-(3,4dichloro-phenyl)-5-hydroxy-2-oxo-pyrrolidine-1-yl]-2-methoxy-phenoxy)-ethyl ester (46 mg, 0.101 mmol) and triethylsilane (1.5 eq, 24 μl) in CH₂Cl₂ (1 ml) was added trifluoroacetic acid (10 eq, 120 μl) dropwise. After stirring for 2 hours at room temperature, the solution was concentrated in vacuo, and then the solution was diluted with water (10 ml) and extracted with ethylacetate (3×10 ml). The combined organic extracts were dried over anhydrous Na₂SO₄, filtered and concentrated to dryness in vacuo. The crude product was purified by trituration in ether/petroleum ether 1/1 to give 30 mg of the title product as a white solid (yield: 69%) (mp: 113° C.)

NMR (¹H, DMSO-d6): δ 7.62 (d, 1H), 7.61 (d, 1H), 7.47 (d, 1H), 7.33 (dd, 1H), 7.09 (dd, 1H), 6.98 (d, 1H), 4.30 (t, 2H), 4.14 (t, 2H), 3.99 (t, 1H), 3.86 (m, 2H), 3.74 (s, 3H), 2.55-2.2 (m+m, 1H+1H), 2.02 (s, 3H). MS (m/z): 375 [MH]⁺, 2Cl.
Preparation 20: 3-(3,4-Dichloro-phenyl)-1-[3-(2-hydroxy-ethoxy)-4-methoxy-phenyl]-pyrrolidine-2-one

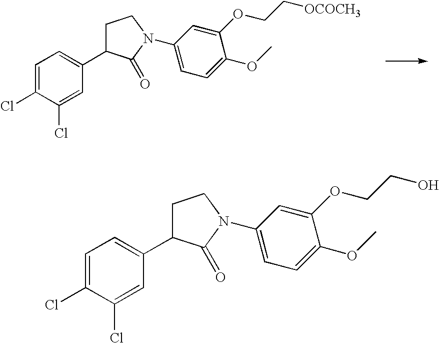

Procedure:

To a solution of acetic acid 2-(5-[3-(3,4-dichloro-phenyl)-2-oxo-pyrrolidine-1-yl]-2-methoxy-phenoxy)-ethyl ester (524 mg, 1.19 mmol) in EtOH 95% (10 ml), at r.t, was added LiOH.H₂O (2 eq., 100 mg). After stirring for 2 hours at room temperature, the solution was concentrated in vacuo, and then diluted with water (10 ml) and extracted with ethylacetate (3×10 ml). The combined organic extracts were dried over anhydrous Na₂SO₄, filtered and concentrated to dryness in vacuo. to give 470 mg of the title product (yield: 99%).

NMR (¹H, DMSO-d6): δ 7.62 (s, 1H), 7.61 (d, 1H), 7.47 (d, 1H), 7.33 (dd, 1H), 7.05 (dd, 1H), 6.96 (d, 1H), 4.82 (t, 1H), 3.99 (t, 1H), 3.92 (t, 2H), 3.86 (m, 2H), 3.70 (t, 2H), 3.74 (s, 3H), 2.55-2.22 (m, 2H). MS (m/z): 396 [MH]⁺+Cl
Preparation 21: 2-(3-Fluoro-phenyl)-pent-4-enoic Acid [3-(2-hydroxy-ethoxy)-4-methoxy-phenyl]-amide

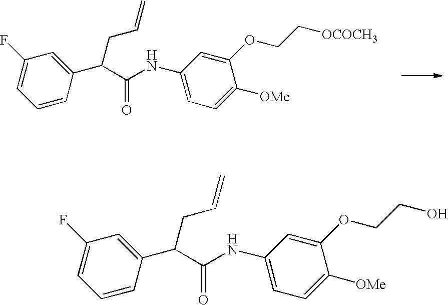

Procedure:

To a solution of a compound of Preparation 15 (0.15 g, 0.37 mmol) in MeOH (1 mL) and THF (1 mL) at 45° C. was added an aqueous solution of KOH (5 M, 0.5 mL). After 15 min the mixture was cooled to 25° C. Aqueous HCl (1 M, 3 mL) and EtOAc were added. The organic layer was collected, dried (MgSO₄), filtered and evaporated in vacuo to give the title compound (0.13 g, 0.36 mmol, 99%) as a colourless solid.

NMR (¹H, CDCl₃, 300 MHz): δ 7.27-7.40 (m, 2H), 6.93-7.15 (m, 4H), 6.71-6.85 (m, 2H), 5.62-5.80 (m, 1H), 4.98-5.11 (m, 2H), 4.05-4.11 (m, 2H), 3.86-3.90 (m, 2H), 3.80 (s, 3H), 3.50 (t, 1H), 2.90-3.02 (m, 1H), 2.46-2.60 (m, 1H), 1.91 (bs, 1H+H₂O). MS (m/z): 360 [MH]⁺.
Preparation 22: Methansulfonic Acid 2-(5-[3-(3,4dichloro-phenyl)-2-oxo-pyrrolidin-1-yl]-2-methoxy-phenoxy)-ethyl Ether

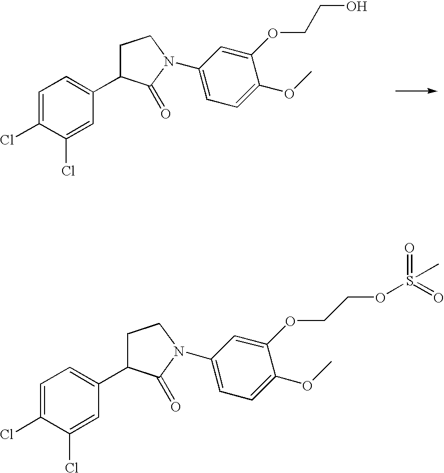

Procedure:

To a solution of alcohol, 3-(3,4-Dichloro-phenyl)-1-[3-(2-hydroxy-ethoxy)-4-methoxy-phenyl]-pyrrolidine-2-one (470 mg, 1.18 mmol) in anh. CH₂Cl₂ (15.6 ml), at 0° C., under N₂, was added Et₃N (5 eq., 822 μl) and CH₃SO₂Cl (2 eq., 180 μl). The reaction was stirred at room temperature for 3 hours. The reaction mixture was diluted with water (20 ml) and extracted with AcOEt (3×25 ml). The combined organic extracts were dried over Na₂SO₄, filtered and concentrated in vacuo concentrated to dryness in vacuo. to give 560 mg of the tile compound as a light yellow foam (yield: 99%).

NMR (¹H, DMSO-d6): δ 7.62 (m, 2H), 7.47 (d, 1H), 7.33 (dd, 1H), 7.13 (dd, 1H), 7.00 (d, 1H), 4.52 (m, 2H), 4.00 (m, 1H), 3.87 (m, 2H), 3.76 (s, 3H), 3.24 (s, 3H), 2.53 (m, 1H), 2.21 (m, 1H). MS (m/z): 474 [MH]⁺+Cl
Preparation 23: Methanesulfonic Acid 2-(5-[2-(3-fluoro-phenyl)-pent-4-enoylamino]-2-methoxy-phenoxy)ethyl Ester

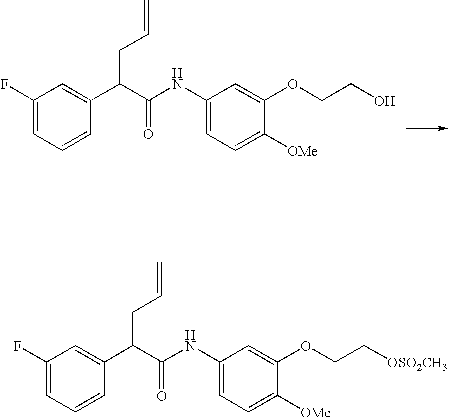

Procedure:

To a partial solution of compound of Preparation 21 (0.13 g, 0.36 mmol) in DCM (2 mL) and THF (2 ml was added N,N-diisopropylethylamine (0.13 mL) followed by methanesulfonyl chloride (0.037 mL) resulting in complete dissolution of the starting material. After 1.5 h water was added with stirring. After an additional 3 min the mixture was partitioned between aqueous HCl (1 M) and a 1:1 mixture of EtOAc and petroleum ether (40-60° C.). The organic layer was washed (water, brine) and the solvents removed in vacuo to give the title product as a slightly yellow film (0.18 g, quant.) containing residual ethyl acetate.

NMR (¹H, CDCl₃, 300 MHz): δ 7.26-7.37 (m, 2H), 7.04-7.16 (m, 3H), 6.94-7.02 (m, 1H), 6.73-6.84 (m, 2H), 5.65-5.80 (m, 1H), 4.97-5.11 (m, 2H), 4.53-4.58 (m, 2H), 4.21-4.26 (m, 2H), 3.77 (s, 3H), 3.50 (t, 1H), 3.13 (s, 3H), 2.87-3.00 (m, 1H), 2.48-2.57 (m, 1H). MS (m/z): 438 [MH]⁺.
Preparation 24: 2-(3-fluoro-phenyl)-pent-4-enoic Acid [4-methoxy-3-(2-piperidin-1-yl-ethoxy)-phenyl]-amide

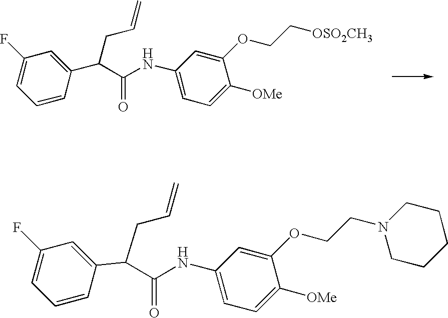

NMR (¹H, CDCl₃): δ 7.25-7.35 (m, 1H), ), 7.10-7.20 (m, 3H), 6.85-7.00 (m, 2H), 6.75 (d, 1H), 5.6-5.8 (m, 1H), 4.9-5.15 (m, 2H), 4.1-4.2 (m, 3H), 3.80 (s, 3H), 3.5 (t, 1H), 2.90-3.05 (m, 2H), ), 2.6-2.75 (m, 2H), 2.5-2.6 (m, 1H), 1.95-2.25 (m, 2H), 1.65-1.85 (m, 4H), 1.4-1.6 (m, 2H). MS (m/z): 427 [MH]⁺.
Preparation 25: 4,4-Difluoro-1-[2-(2-methoxy-4-nitro-phenoxy)-ethyl]-piperidine

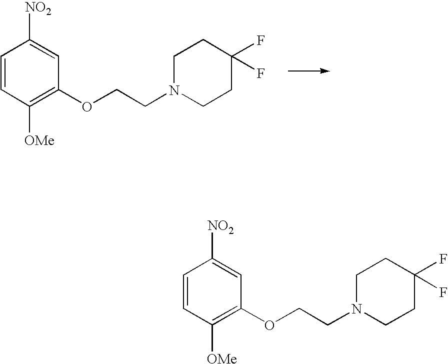

Procedure:

1-(4,4-Difluoro-piperidine-1-yl)-2-(2-methoxy-4-nitro-phenoxy)-ethanone (320 mg, 0.96 mmol) was dissolved in anh. THF (20 ml), 1M BH₃.THF (2.2 eq, 2.4 ml) was added dropwise to the solution, and the mixture was heated at reflux for 5 hours. The solution was cooled to room temperature, CH₃OH (10 ml) was added, and the solvent was removed under reduced pressure. The residue was dissolved in CH₃OH (10 ml), 6N HCl (20 ml) was added, and the solution was heated to reflux for 1 hour. The CH₃OH was removed under reduced pressure and the remaining aqueous solution was made basic (pH>10) with 2.5N NaOH. The basic solution was extracted with ethyl acetate (3×20 ml). The combined organic extracts were dried over anhydrous Na₂SO₄ and the solvent was removed under reduced pressure to give 258 mg of the title product as a light yellow oil. (yield: 85%)

NMR (¹H. CDCl₃): δ 7.91 (dd, 1H), 7.77 (d, 1H), 6.89 (d, 1H), 4.19 (t, 2H), 2.90 (t, 2H), 2.71 (m, 4H), 2.01 (m, 4H).
Preparation 26: 4-[2-(4,4Difluoro-piperidin-1-yl)-ethoxy]-3-methoxy-phenylamine

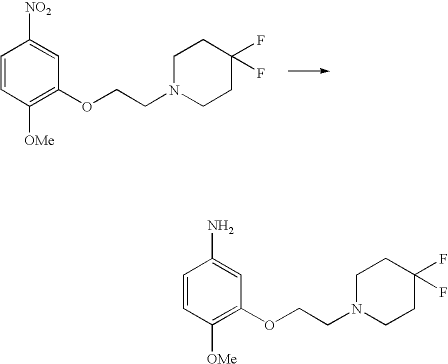

NMR (¹H, CDCl₃): δ 6.73 (d, 1H), 6.34 (d, 1H), 6.29 (dd, 1H), 4.18 (t, 2H), 3.78 (s, 3H), 3.02 (t, 2H), 2.89 (bs, 4H), 2.13 (m, 4H).
Preparation 27: 4-Methoxy-3-(2-pyrrolidin-1-yl-ethoxy)-phenylamine

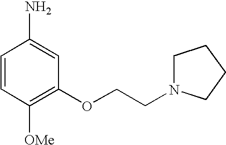

The title compound (brown solid) was prepared in an analogous manner to Preparations 9 and 11 using 2-methoxy-5-nitrophenol and 1-(2-chloroethyl)pyrrolidine hydrochloride.

NMR (¹H, CDCl₃): δ 6.70 (d, 1H), 6.30 (s, 1H), 6.20 (d, 1H), 4.08 (t, 2H), 3.75 (s, 3H), 3.40 (bs, 2H), 2.90 (t, 2H), 2.65-2.55 (m, 4H), 1.80-1.74 (m, 4H). MS (m/z): 237 [MH]⁺.
Preparation 28: 2-(3,4-Dichloro-phenyl)-pent-4-enoic-acid [4-methoxy-3-(2-pyrrolidin-1yl-ethoxy)-phenyl]-amide

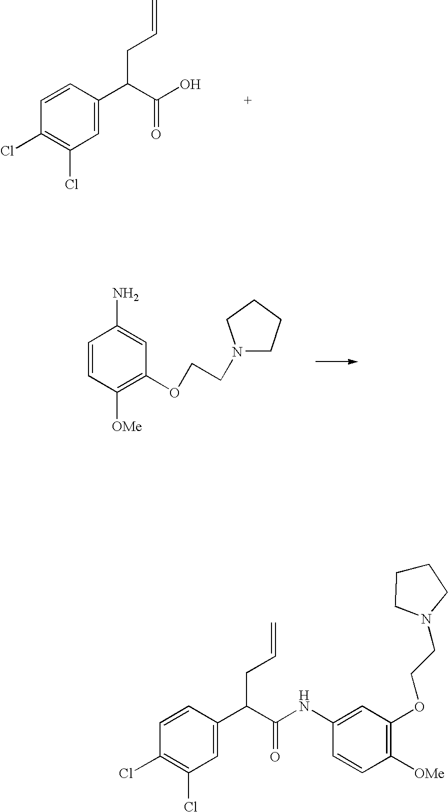

To a solution of 2-(3,4-dichloro-phenyl)-pent-4-enoic acid (0.19 g) in dry CH₂Cl₂ (3 ml), under N₂, was added, at 0° C., oxalyl chloride (0.10 ml) and DMF (cat). The reaction was allowed to warm to room temperature. After 1 h the reaction mixture was concentrated to dryness in vacuo. To this material was added a DMF (dry, 2 ml) solution of 4-methoxy-3-(2-pyrrolidin-1-yl-ethoxy)-phenylamine (0.22 g) which had been converted to the hydrochloride salt by treatment with HCl/Et₂O (1 eq.) followed by evaporation. The mixture was heated at 110° C. for 2 h, then partitioned between aqueous NaHCO₃ and EtOAc. The organic layer was washed (brine), concentrated and purified by column chromatography (silica gel, CH₂Cl₂/MeOH/NH₃) to give the title compound (0.18 g) as an orange oil.

NMR (¹H, CDCl₃): δ 7.50-7.35 (m, 3H), 7.15 (s, 1H), 6.90 (d, 1H), 6.73 (d, 1H), 5.77-5.62 (m, 1H), 5.10-4.96 (m, 2H), 4.15-4.05 (m, 2H), 3.80 (s, 3H), 3.46 (t, 1H), 2.96-2.83 (m, 3H), 2.70-2.54 (m, 4H), 2.40 (bs, 2H), 1.85-1.73 (m, 4H). MS (m/z): 463 [MH](⁺, 2Cl.
Preparation 29: 3-(3,4-Dichloro-phenyl)-5-hydroxy-1-[4-methoxy-3-(2-pyrrolidin-1-yl-ethoxy)-phenyl]-pyrrolidin-2-one

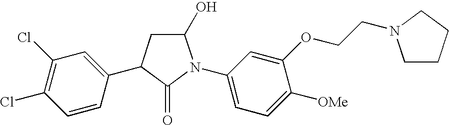

The title compound (colourless foam, ca. 1:1 mixture of diastereoisomers) was prepared from 2-(3,4dichloro-phenyl)-pent-4-enoic-acid [4-methoxy-3-(2-pyrrolidin-1-yl-ethoxy)-phenyl]-amide in an analogous manner to the one described for Example 15.

NMR (¹H, CDCl₃): δ 7.44 (s, 0.5H), 7.28-7.12 (m, 2H), 7.06 (s, 0.5H), 6.95-6.80 (m, 2H), 6.68 (d, 1H), 5.54-5.44 (m, 1H), 3.96-3.85 (m, 3H), 3.67 (s, 3H), 3.60-3.53 (m, 0.5H), 3.78-3.70 (m, 2.5H), 2.47-2.18 (m, 5.5H), 2.00-1.91 (m, 0.5H), 1.67-1.55 (m, 4H). MS (m/z): 465 [MH]⁺, 2Cl.
Preparation 30: [4-Methoxy-3-(2-piperidin-1-yl-ethoxy)-phenyl]-acetic Acid 2-piperidin-1-yl-ethyl Ester

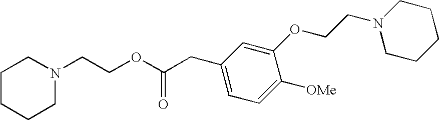

A mixture of (3-hydroxy-4-methoxy-phenyl)-acetic acid (1.85 g), dry DMF (25 ml), K₂CO₃ (5.9 g) and N-chloroethylpiperidine hydrochloride (3.74 g) was heated at 40° C. for 5 h. Volatiles were then removed in vacuo and the residue partitioned between water and EtOAc. The organic layer was washed (brine) and concentrated to give the title (3.76 g) compound as an orange oil.

NMR (¹H, CDCl₃): δ 6.93-6.80 (m, 3H), 4.22 (t, 2H), 4.14 (t, 2H), 3.82 (s, 3H), 3.55 (s, 2H), 2.82 (t, 2H), 2.66-2.40 (m, 10H), 1.66-1.40 (m, 12H).
Preparation 31: [4-Methoxy-3-(2-piperidin-1-yl-ethoxy)-phenyl]-acetic Acid Methyl Ester

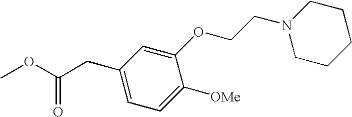

[4-Methoxy-3-(2-piperidin-1-yl-ethoxy)-phenyl]-acetic acid 2-piperidin-1-yl-ethyl ester (6.3 g) in MeOH (6 ml), THF (6 ml) and water (6 ml) containing KOH (1.7 g) was heated at 45° C. for 1 h and then allowed to cool to 25° C. over 90 min. With stirring in an ice bath conc. aqueous HCl (6 ml) was then added. The mixture was evaporated to dryness. The material was heated at reflux with HCl in MeOH (1 M) for 4 h, concentrated and extracted with DCM. The mixture was filtered and the solvent removed in vacuo to give the title compound (4.4 g) as an orange oil.

NMR (¹H, CDCl₃): δ 6.88-6.79 (m, 3H), 4.18 (t, 2H), 3.84 (s, 3H), 3.69 (s, 3H), 3.55 (s, 2H), 2.87 (t, 2H), 2.56 (bs, 4H), 1.66 (bs, 4H), 1.46 (bs, 2H).
Preparation 32: 2-[4-Methoxy-3-(2-piperidin -1-yl-ethoxy)-phenyl]-pent-4-enoic Acid Methyl Ester

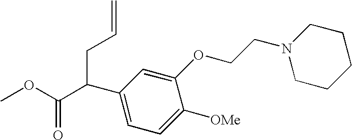

Procedure:

To a solution of [4-methoxy-3-(2-piperidin-1-yl-ethoxy)-phenyl]-acetic acid methyl ester (2.1 g) in THF (dry, 15 ml) at −78° C. was slowly added lithium bis(trimethylsilyl)amide (1 M in THF, 8.2 ml). The solution was stirred at this temperature for 15 min before allyl bromide (0.59 ml) was added. After additional 30 min water and EtOAc were added with stirring. The mixture was allowed to warm to 25° C., layers separated and the organic layer washed (brine), concentrated and submitted to column chromatography (silica gel, CH₂Cl₂/MeOH/NH₃) to give the title compound (1.3 g) as a colourless oil.

NMR (¹H, CDCl₃): δ 6.90-6.80 (m, 3H), 5.76-5.67 (m, 1H), 5.10-4.98 (m, 2H), 4.18 (bs, 2H), 3.83 (s, 3H), 3.65 (s, 3H), 3.56 (t, 1H), 2.86 (bs, 2H), 2.83-2.73 (m, 1H), 2.65-2.45 (m, 5H), 1.65 (bs, 4H), 1.47 (bs, 2H). MS (m/z): 348 [MH]⁺.
Preparation 33: 2-[4-Methoxy-3-(2-piperidin-1-yl-ethoxy)-phenyl]-pent-4-enoic Acid Hydrochloride Salt

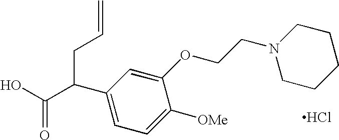

2-[4Methoxy-3-(2-piperidin-1-yl-ethoxy)-phenyl]-pent-4-enoic acid methyl ester (1.3 g) in MeOH (2 ml), THF (2 ml) and water (2 ml) containing KOH (0.42 g) was heated at 45° C. for 1 h and then allowed to cool to 25° C. The mixture was evaporated to dryness. THF (5 ml) and conc. aqueous HCl (0.62 ml) were added, the mixture concentrated, extracted with DCM, filtered and the solvent removed in vacuo to give the title compound (1.2 g) as an off-white foam.

NMR (¹H, CD₃OD): δ 7.05 (d, 1H), 6.96 (dd, 1H), 6.91 (d, 1H), 5.81-5.70 (m, 1H), 5.02 (dd, 1H), 4.92 (dd, 1H), 4.31 (dd, 2H), 3.83 (s, 3H), 3.48-3.42 (m, 3H), 3.37-3.28 (m, 4H), 2.78-2.69 (m, 1H), 2.44-2.35 (m, 1H), 1.91-1.84 (m, 4H), 1.72-1.63 (m, 2H). MS (m/z): 334 [MH]⁺.
Preparation 34: 2-[4-Methoxy-3-(2-piperidin-1-yl-ethoxy)-phenyl]-pent-enoic Acid (3,4-dichlorophenyl)-amide

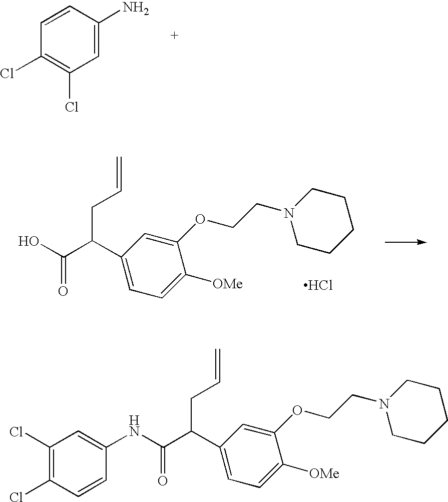

To a solution of 2-[4-methoxy-3-(2-piperidin-1-yl-ethoxy)-phenyl]-pent-4-enoic acid hydrochloride salt (0.46 g) in dry CH₂Cl₂ (4 ml), under N₂, was added, at 0° C., oxalyl chloride (0.11 ml) and DMF (cat). After 30 min the reaction mixture was concentrated to dryness in vacuo. To this material was added toluene (dry, 4 ml) and 3,4-dichloroaniline (0.20 g). The mixture was heated at 105° C. for 4 h, then partitioned between aqueous NaHCO₃ and EtOAc. The organic layer was washed (brine), concentrated and purified by column chromatography (silica gel, CH₂Cl₂/MeOH/NH₃) to give the title compound (0.35 g) as a slightly brown oil.

NMR (¹H, CDCl₃): δ 7.74 (s, 1H), 7.55 (bs, 1H), 7.31 (bs, 2H), 6.96 (bs, 1H), 6.91-6.83 (m, 2H), 5.77-5.68 (m, 1H), 5.09 (d, 1H), 5.01 (d, 1H), 4.23-4.16 (m, 2H), 3.85 (s, 3H), 3.53 (t, 1H), 2.99-2.91 (m, 1H), 2.83 (t, 2H), 2.55 (bs, 4H), 1.68-1.62 (m, 4H), 1.47 (bs, 3H). MS (m/z): 477 [MH]⁺, 2Cl.
Preparation 35: 2-[4-Methoxy-3-(2-piperidin-1-yl-ethoxy)-phenyl]-propanoic Acid Methyl Ester

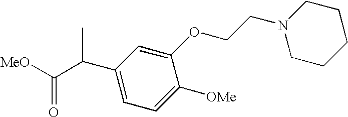

To a solution of [4-methoxy-3-(2-piperidin-1-yl-ethoxy)-phenyl]-acetic acid methyl ester (0.60 g) in THF (dry, 6 ml) at −78° C. was slowly added lithium bis(trimethylsilyl)amide (1 M in THF, 2.3 ml). The solution was stirred at this temperature for 15 min before iodomethane (0.12 ml) was added, then allowed to warm to 25° C. After 16 aqueous NaHCO₃ and EtOAc were added with stirring, layers separated and the organic layer washed (brine), concentrated and submitted to column chromatography (silica gel, CH₂Cl₂/MeOH/NH₃) to give the title compound (0.27 g) as a slightly yellow oil.

NMR (¹H, CD₃OD): δ 6.88-6.75 (m, 3H), 4.12 (t, 2H), 3.80 (s, 3H), 3.67-3.34 (m, 4H), 2.78 (t, 2H), 2.50 (bs, 4H), 1.65-1.37 (m, 9H). MS (m/z): 322 [MH]⁺.
Preparation 36: 2-(3-Chloro-phenyl)-pent-4-enoic Acid {3-[2-(4-methyl-piperidin-1-yl)-ethoxy]-4-methoxy-phenyl}-amide

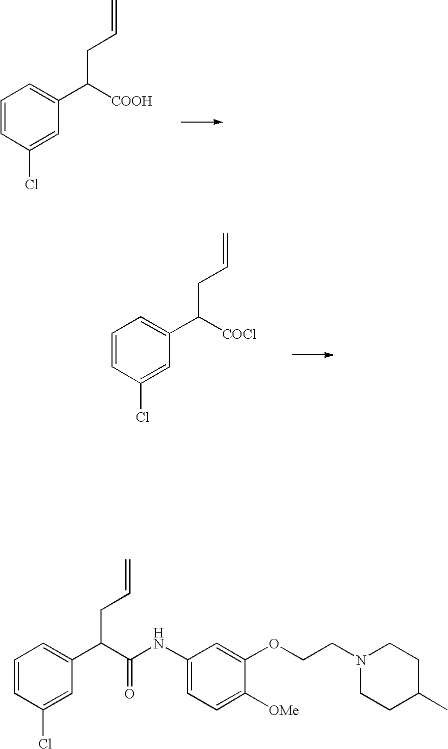

The title compound was prepared, in an analogous manner to the compound of Preparation 14, in 317 mg yield of brow oil (y=73%) from 2-(3,4-dichloro-phenyl)-pent-4-enoic acid (200 mg).

MS (m/z): 457 [MH]⁺, 1Cl.
Preparation 37: 3-(3Chloro-phenyl)-5-hydroxy-1-{4-methoxy-3-[2-(4-methyl-piperidin-1-yl)-ethoxy]-phenyl}-pyrrolidin-2-one

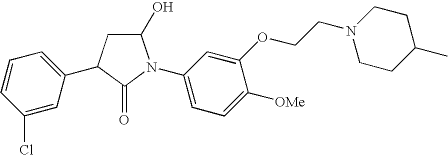

The title compound was prepared, in an analogous manner to the compound of Example 15, in 176 mg yield as brow oil (y=55%) from 2-(3chloro-phenyl)-pent-4-enoic acid {4-methoxy-3-[2-(4-methylpiperidin)-1-yl-ethoxy]-phenyl}-amide (317 mg).

MS (m/z): 459 [MH]⁺, 1Cl.
Preparation 38: 5-(tert-Butyl-dimethyl-silanyloxy)-2-(3,4-dichloro-phenyl)-pentanoic Acid

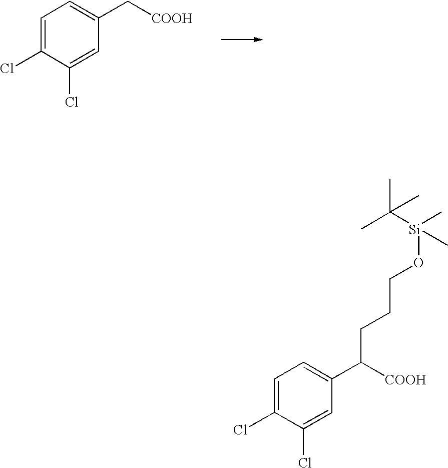

Procedure:

A solution of (3,4-Dichloro-phenyl)-acetic acid (1.5 g, 7.3 mmol) in anh. THF (15 ml), under N₂, was treated with lithium bis(trimethylsilyl) amide (1M solution in THF, 2.2 eq., 16 ml) at −78° C. for 30 minutes before (3-bromo-propoxy)-tert-butyl-dimethyl-silane (1.7 eq., 2.9 ml) was added. The mixture was stirred for 2 hours at room temperature and quenched with saturated solution of Ammonium Chloride (20 ml). The product was extracted with ethyl acetate (2×15 ml) and the organic phase was washed with brine (1×15 ml), dried over Na₂SO₄ and concentrated in vacuo. The crude product was purified by flash chromatography (silica gel, AcOEt/cHex 1/1 then AcOEt) to give 16 g of the title product as a pale yellow oil (56%).

NMR (¹H, DMSO-d6): δ 12.6 (sb, 1H), 7.58 (d, 1H), 7.53 (d, 1H), 7.27 (dd, 1H), 3.59 (t, 1H), 3.54 (t, 2H), 2.00 (m, 1H), 1.7 (m, 1H), 1.4 (m, 1H), 1.3 (m, 1H), 0.82 (s, 9H), −0.01 (s, 3H), −0.02 (s, 3H).
Preparation 39: 5-(tert-Butyl-dimethyl-silanyloxy)-2-(3,4-dichloro-phenyl)-pentanoic Acid [4-methoxy-3-(2-piperidin-1-yl-ethoxy)-phenyl]-amide

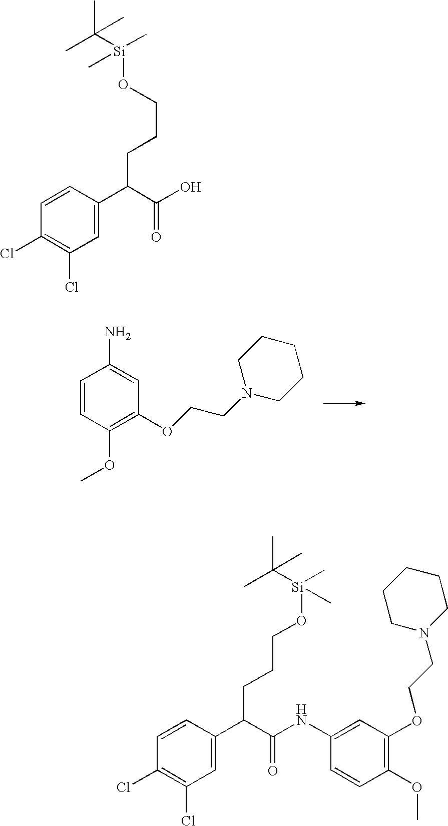

Procedure:

To a solution of 5-(tert-butyl-dimethyl-silanyloxy)-2-(3,4-dichloro-phenyl)-pentanoic acid (780 mg, 2.1 mmol) in anh. DMF (5 ml), under N₂, was added 4methoxy-3-(2-piperidin-1-yl-ethoxy)-phenylamine (1 eq., 523 mg) followed by 1-hydroxybenzotriazole (1.2 eq., 360 mg) and 1-(dimethylaminopropyl)-3-ethylcarbodiimide hydrochloride (1.2 eq., 476 mg). The reaction was stirred at room temperature for 16 h. AcOEt was added (10 ml) and the organic phase was washed with H₂O (10 ml), NaHCO₃ sat. (10 ml) brine (10 ml), dried over Na₂SO₄ and concentrated in vacuo to obtain the title product as a pale yellow foam 1.25 g (98%).

NMR (¹H, CDCl₃): δ 7.75 (s, 1H), 7.45 (s, 1H), 7.3 (d, 1H), 7.2 (m, 1H), 7.00 (d, 1H), 6.7 (d, 1H), 4.1 (m, 2H), 3.75 (s, 2H), 3.5 (m, 2H), 2.6 (m, 4H), 1.6-1.3 (m, 8H), 0.9 (s, 9H), 0.5 (s, 6H)
Preparation 40: 2-(3,4dichloro-phenyl)-5-hydroxy Pentanoic Acid [4-methoxy-3-(2-piperidin-1yl-ethoxy)-phenyl]-amide

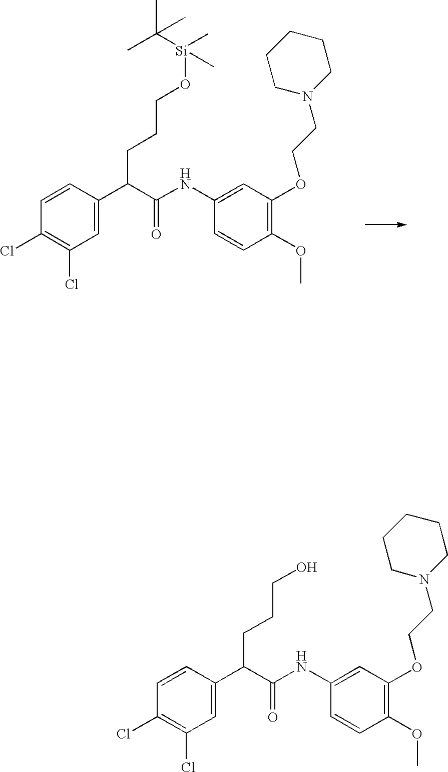

Procedure:

To a solution of 5-(tert-butyl-dimethyl-silanyloxy)-2-(3,4-dichloro-phenyl)-pentanoic acid [4-methoxy-3-(2-piperidin-1-yl-ethoxy)-phenyl]-amide (780 mg, 2.1 mmol) in anh. DMF (2 ml), under N₂, triethylamine trihydrofluoride (0.5 eq., 0.17 ml) was added. The reaction was stirred at room temperature for 18 h. AcOEt was added (10 ml) and the organic phase was washed with NaHCO₃ sat (10 ml), dried over Na₂SO₄ and concentrated in vacuo to obtain the title product as a white foam 1 g (100%).

NMR (¹H, DMSO-d6): δ 9.98 (s, 1H), 7.6 (m, 2H), 7.34 (d, 1H), 7.29 (d, 1H), 7.07 (d, 1H), 7.2 (m, 1H), 6.85 (d, 1H), 4.42 (t, 1H), 3.98 (t, 2H), 3.69 (s, 3H), 3.64 (t, 1H), 3.39 (q, 2H), 2.66 (bs, 2H), 2.46 (bm, 4H), 2.0 (m, 1H), 1.7 (m, 1H), 1.49 (bm, 4H), 1.37 (m, 2H), 1.33 (m, 2H). MS (m/z): 495 [MH]⁺, 2Cl.
Preparation 41: 2-Chloro-1-(4-metyl-piperidin-1-yl)-ethanone

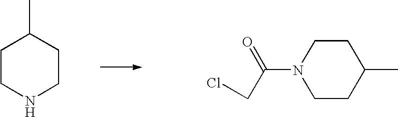

Procedure:

To a solution of 4-metylpiperidine (10 g, 100 mmol) in anh. CH₂Cl₂ (100 ml) was added, at 0° C. and under N₂, TEA (2.5 eq, 35 ml) and chloroacetyl chloride (1.2 eq, 9.6 ml). The reaction was stirred at room temperature for 6 hours. The solution was diluted with water (60 ml) and extracted with ethyl acetate (3×100 ml). The combined organic extracts were dried over anhydrous Na₂SO₄, filtered and concentrated to dryness in vacuo to give 13.6 g of the title product (yield: 78%).

NMR (¹H, CDCl₃): δ 4.57 (d, 1H), 4.11 (d, 2H), 3.82 (d, 1H), 3.12 (t, 1H), 2.67 (t, 1H), 1.67 (m, 4H), 1.22 (m, 1H), 1.02 (d, 3H). MS (m/z): 176 [MH]⁺, 1Cl
Preparation 42: 2-(2-Methoxy-5-nitro-phenoxy)-1-(4-methyl-piperidin-1-yl)-ethanone

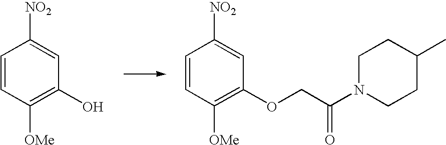

Procedure:

To a solution of 2-methoxy-5-nitrophenol (350 mg, 2.06 mmol) in DMF (11 ml) at room temperature were added K₂CO₃ (312 mg, 2.2 mmol) and 2-chloro-1-(4-methyl-piperidin-1-yl)-ethanone (396 mg, 2.2 mmol). The suspension was stirred at room temperature for 24 hours. The suspension was diluted with water (20 ml) and extracted with EtOAc (2×20 ml), the combined organic phases were washed with NaOH 1N (3×20 ml), dried over Na₂SO₄, filtered and concentrated to dryness in vacuo, to obtain the title product as a red oil (0.38 g, 58%)

NMR (¹H, CDCl₃): δ 8.00 (dd, 1H), 7.76 (d, 1H), 6.98 (d, 1H), 4.87 (s, 3H), 4.55 (d, 1H), 4.02 (s, 3H), 3.85 (d, 1H), 3.13 (t, 1H), 2.68 (t, 1H), 1.68 (m, 2H), 1.27 (m, 2H), 1.01 (d, 3H). MS (m/z): 309 [MH]⁺.
Preparation 43: 1-[2-(2-Methoxy-5-nitro-phenoxy)-ethyl]-4-methyl-piperidine 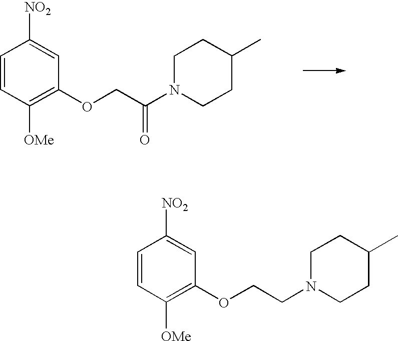
Procedure:

2-(2-Methoxy-5-nitro-phenoxy)-1-(4-methyl-piperidin-1-yl)-ethanone (11 g, 36 mmol) was dissolved in anh. THF (20 ml), 1M BHₛTHF (2.2 eq, 78.5 ml) was added dropwise to the solution, and the mixture was heated at reflux for 4 hours. The solution was cooled to room temperature, CH₃OH (100 ml) was added, and the solvent was removed under reduced pressure. The residue was dissolved in CH₃OH (100 ml) , 6N HCl (200 ml) was added, and the solution was heated to reflux for 0.5 h. The CH₃OH was removed under reduced pressure and the remaining aqueous solution was made basic (pH>10) with 2.5N NaOH. The basic solution was extracted with ethyl acetate (3×80 ml). The combined organic extracts were dried over anhydrous Na₂SO₄ and the solvent was removed under reduced pressure to give 9.4 g of the title product as a red oil. (yield: 89%)

NMR (¹H, CDCl₃): δ 7.91 (dd, 1H), 7.80 (d, 1H), 6.90 (d, 1H), 4.21 (t, 2H), 3.96 (s, 3H), 2.97 (m, 2H), 2.85 (t, 2H), 2.12 (m, 2H), 1.64 (m, 2H), 1.4 (m, 1H), 1.29 (m, 2H), 0.92 (d, 3H). MS (m/z): 295 [MH]⁺.
Preparation 44: 4-Methoxy-3-[2-(4-methyl-piperidin-1-yl)-ethoxy]-phenylamine

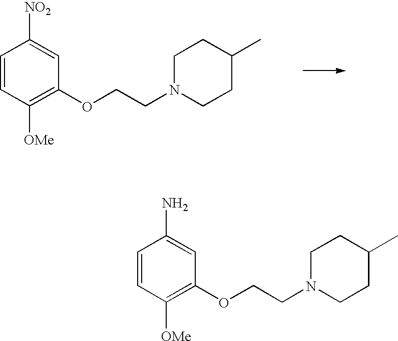

Procedure:

To a solution of 1-[2-(2-methoxy-5-nitro-phenoxy)-ethyl]-4-methyl-piperidine (9.5 g, 32.3 mmol) in abs.EtOH (200 ml) were added Pd/C 10% (1 g) and the reaction mixture was hydrogenated at room temperature and at atmospheric pressure. After 5 hrs the reaction mixture was filtered through a celite pad and the solution was concentrated in vacuo to give 8.5 g of the title product as a brown solid (yield: 99%).

NMR (¹H, DMSO-d₆): δ 6.61 (d, 1H), 6.26 (d, 1H), 6.04 (dd, 1H), 4.60 (bs, 2H), 3.91 (t, 2H), 3.58 (s, 3H), 2.85 (m, 2H), 2.61 (t, 2H), 1.97 (m, 2H), 1.52 (m, 2H), 1.3 (m, 1H), 1.1 (m, 2H), 0.86 (d, 3H).

Preparation 45: 2-(3,4-Dichloro-phenyl)-pent-4-enoic acid (4-methoxy-3-[2-(4-methyl-piperidin-1-yl)-ethoxy]-phenyl)-amide

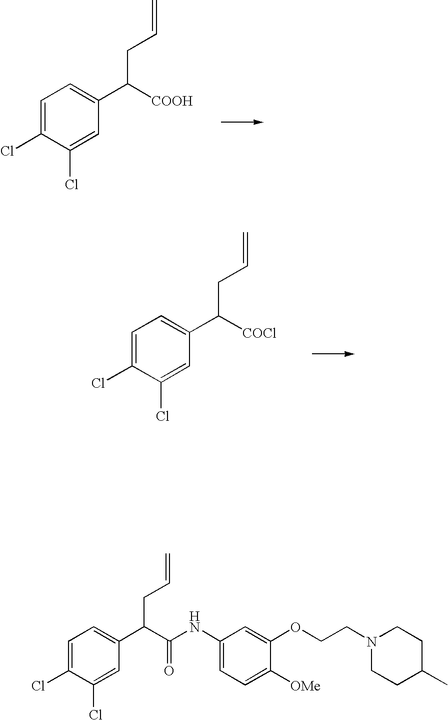

The title compound was prepared in an analogous manner to the compound of preparation 14

NMR (¹H, CDCl₃): δ 7.50 (bs, 1H), 7.43 (d, 1H), 7.25 (dd, 1H), 7.21 (bs, 1H), 6.92 (dd, 1H), 6.78 (d, 1H), 5.74 (m, 1H), 5.13 (d, 1H), 5.03 (d, 1H), 4.14 (t, 2H), 3.81 (s, 3H), 3.48 (t, 1H), 3.03 (d, 2H), 2.95 (m, 1H), 2.86 (t, 2H), 2.55 (m, 1H), 2.18 (t, 2H), 1.65 (d, 2H), 1.4 (m, 1H), 1.32 (t, 1H), 0.94 (d, 3H). MS (m/z): 491[MH]⁺, 2Cl.
Preparation 46: 3-(3,4Dichloro-phenyl)-5-hydroxy-1-(4-methoxy-3-[2-(4-methyl-piperidin-1-yl)-ethoxy]-phenyl)-pyrrolidin-2-one

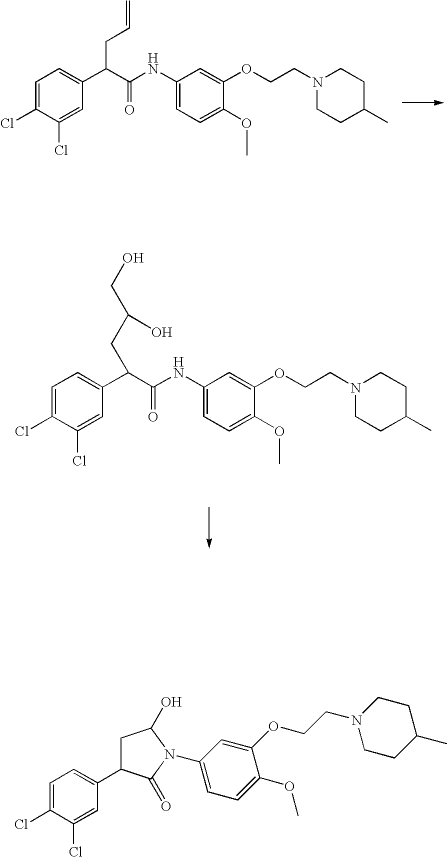

rocedure

To a solution 2-(3,4dichloro-phenyl)-pent-4-enoic add (4-methoxy-3-[2-(4-methyl-piperidin-1-yl)ethoxy]-phenyl)-amide (0.25 g, 0.51 mmol) in acetone/H₂O 8/1 (9.1/1.1 ml) was added N-methyl-morpholine-N-oxide (2 eq, 120 mg) and OSO₄ 4 wt % sol. in water (cat., 100 μl). The reaction was stirred at room temperature for 18 hours and then quenched with 10 ml of Na₂SO₃ sat. After 15 minutes stirring the diol was extracted with ethyl acetate (2×20 ml), dried over Na₂SO₄, filtered and concentrated to dryness in vacuo. The crude product was then dissolved in THF/H₂O 1/1 (515 ml) and potassium periodate (1.5 eq, 0.16 g) was added. The reaction was stirred at room temperature for 2 hours. The solution was diluted with water (10 ml) and extracted with ethyl acetate (3×10 ml). The combined organic extracts were dried over anhydrous Na₂SO₄ filtered and concentrated to dryness in vacuo. Flash chromatography of the crude product (silica gel, CH₂Cl₂/CH₃OH 4/1) gave 0.14 g of the title product as a red oil. (yield: 57% in 2 steps)

NMR (¹H, DMSO-d6): δ 7.61 (m, 2H), 7.32 (dd, 1H), 7.25 (bs, 1H), 7.07 (dd, 1H), 6.95 (d, 1H), 6.45 (d, 1H), 5.61 (m, 1H), 4.20 (t, 1H), 4.01 (t, 2H), 3.74 (s, 3H), 2.67 (t, 2H), 2.90 (d, 2H), 2.45 (m, 1H), 2.35 (m, 1H), 2.20 (t, 2H), 1.56 (d, 2H), 1.23 (t, 2H), 1.3 (m, 1H), 0.87 (d, 3H).

MS (m/z): 493 [MH]⁺, 2Cl.
Preparation 47: (3,4-Dichloro-phenyl)-acetic acid but-2-enyl Ester

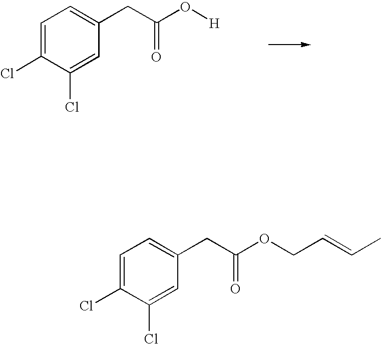

Procedure: To a solution of 3,4-dichloro-phenylacetic acid (2 g, 9.7 mmol) in anh. CH₂Cl₂ (40 ml), under N₂, was added, at 0° C., oxalyl chloride (1.9 eq., 1.6 ml) and DMF (cat). The reaction was stirred at room temperature for 3 hours. The reaction mixture was concentrated to dryness in vacuo and the crude intermediate was then dissolved, under N₂, in anh. CH₂Cl₂ (40 ml). Crotyl alcohol (2 eq, 1.7 ml) and pyridine (1.1 eq., 1.2 ml) were added to the reaction mixture at room temperature. The reaction was stirred at room temperature for 18 hours and then quenched with ammonium chloride (40 ml). The product was extracted with ethyl acetate (3×25 ml) dried over Na₂SO₄, filtered and concentrated to dryness in vacuo. The crude product was purified by flash chromatography (silica gel, AcOEt/cHex 19/1) to give 1.8 g of the title product as a pale yellow oil (72%).

NMR (¹H, DMSO-d₆): 7.57 (d, 1H), 7.56 (bs, 1H), 7.28 (dd, 1H), 5.74 (m, 1H), 5.57 (m, 1H), 4.49 (d, 2H), 3.73 (s, 2H), 1.67 (d, 3H).
Preparation 48: 2(3,4-Dichloro-phenyl)-3-methyl-pent-4-enoic Acid

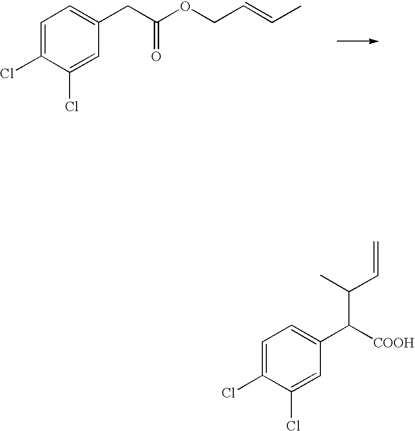

Procedure:

To a stirred solution of lithium diisopropylamide (LDA) (1.8 ml, 3.5 mmol) in anh. THF (2 ml) was added via a syringe, a solution of the ester (3,4-dichloro-phenyl)-acetic acid but-2-enyl ester (0.38 g, 1.43 mmol) in THF (2 ml). After 30 min the solution was treated with trimethylsilyl chloride (0.45 mmol, 2.5 ml) and after a further 30 min at −78° C., the mixture was warmed to ambient temperature during 30 min which was stirred to and heated at reflux for 2 hrs. It was then cooled to ambient temperature before the addition of water (5 ml) and acidification to pH 2 using HCl 1M. The product was extracted into ethyl acetate (3×10 ml). The combined organic extracts were dried over anhydrous Na₂SO₄ and the solvent was removed under reduced pressure. Flash chromatography of the crude product (cHex/EA 411 to 100% EA) gave 0.14 g of the title product as a brown oil. (yield: 38%) which is a mixture of diastereoisomers A/B 50/50.

NMR (¹H, DMSO-d₆): 12.62 (bs, 1H), 7.55 (m, 2H), 7.3 (dd, 1H), 5.81 (m, 1H_{A}), 5.45 (m, 1H_{B}), 5.01 (dd, 2H_{A}), 4.81 (dd, 2H_{B}), 3.43 (d, 1H), 2.78 (m, 1H), 1.06 (d, 3H_{A}), 0.74 (d, 3H_{B}).
Preparation 49: 2-(3,4-Dichloro-phenyl)-3-methyl-pent-4-enoic Acid (4-methoxy-3-[2-(4-piperidin-1-yl)-ethoxy]-phenyl)-amide

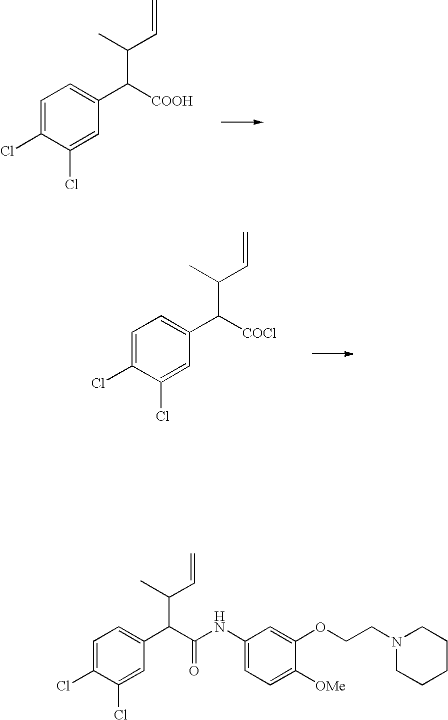

The title compound was prepared in an analogous manner to the compound of Preparation 14 mixture of diastereoisomers: A/B 60/40

NMR (¹H, DMSO-d₆): δ 10.05 (s, 1H_{B}), 9.94 (s, 1H_{A}), 7.65-7.50 (m, 2H), 7.38 (dd, 1H_{A}), 7.34 (dd, 1H_{B}), 7.27 (bs, 1H_{B}), 7.23 (bs, 1H_{A}), 7.06 (dd, 1H_{B}), 7.04 (dd, 1H_{A}), 6.85 (m, 1H), 5.84 (m, 1H_{A}), 5.50 (m, 1H_{B}), 5.10-5.00 (m, 2H_{A}), 4.88-4.82 (m, 2H_{B}), 3.97 (bs, 2H), 3.69 (s, 3H_{B}), 3.68 (s, 3H_{A}), 3.43 (m, 1H), 2.90 (m, 1H), 2.63 (bs, 2H), 2.41 (bs, 4H), 1.48 (bs, 4H), 2.41 (bs, 2H), 1.07 (3H_{B}), 0.76 (3H_{A}). MS (m/z): 491 [MH]⁺, 2Cl
Preparation 50: 2-(4-Chloro-phenyl)-pent-4-enoic Acid (4-methoxy-3-[2-(4-methyl-piperidin-1-yl)-ethoxy]-phenyl)-amide

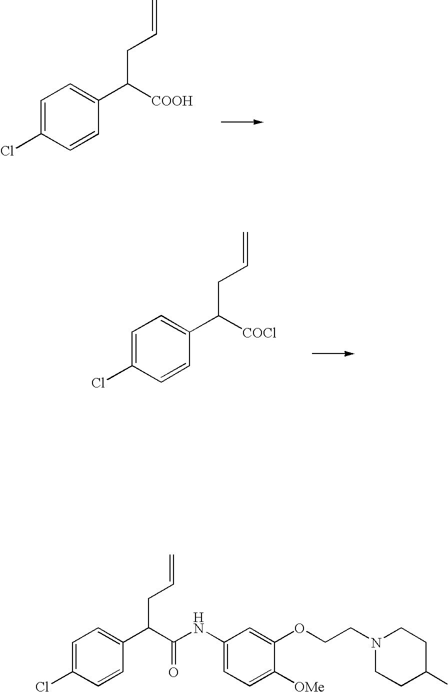

The title compound was prepared in an analogous manner to the compound of Preparation 14

NMR (¹H, DMSO-d₆): δ 9.94 (S, 1H), 7.38 (m, 4H), 7.26 (d, 1H), 7.05 (dd, 1H), 6.84 (d, 1H), 5.68 (m, 1H), 5.0 (dd, 2H), 3.95 (t, 2H), 3.70 (t, 1H), 3.68 (S, 3H), 2.63 (t, 2H), 2.55 (m, 2H), 2.85 (m, 2H), 1.97 (m, 2H), 1.531.08 (m, 4H), 1.3 (m, 1H), 0.86 (d, 3H). MS (m/z): 457 [MH]⁺, 1Cl.
Preparation 51: 2-(4-Chloro-phenyl)-pent-4-enoic Acid (4methoxy-3-[2-(4-piperidin-1-yl)-ethoxy]-phenyl)-amide

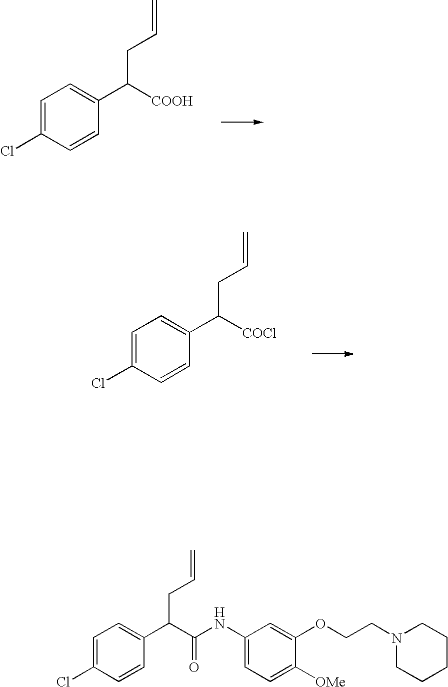

The title compound was prepared in an analogous manner to the compound of Preparation 14

NMR (¹H, DMSO-d₆): δ 9.95 (s, 1H), 7.38 (m, 4H), 7.26 (d, 1H), 7.05 (dd, 1H), 6.84 (d, 1H), 5.7 (m, 1H), 5.0 (dd, 2H), 3.95 (t, 2H), 3.70 (t, 1H), 3.68 (s, 3H), 2.61 (t, 2H), 2.5 (m, 2H), 2.40 (m, 4H), 1.53-1.3 (m, 6H). MS (m/z): 443 [MH]⁺, 1Cl.
Preparation 52: 3-(4-Chloro-phenyl)-5-hydroxy-1-(4-methoxy-3-[2-(4methyl-piperidin-1-yl)-ethoxy]-phenyl)-pyrrolidin-2-one

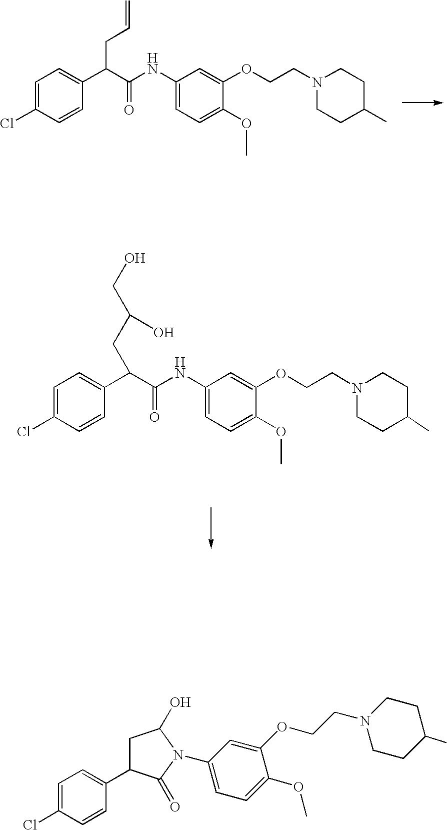

The title compound was prepared in an analogous manner to the compound of Preparation 18.

Two isomers A) and B) are present in 54/46 ratio.

NMR (¹H, DMSO-d6): δ 7.4-7.31 (d+d, 4H), 7.45-6.95 (m, 3H), 6.5-6.4 (d, 1H), 5.65 (m, 1H), 4.00 (t, 2H), 2.67 (t, 2H), 4.12-3.8 (m, 1H), 3.74 (s, 3H) 2.87-2.0 (m, 2H), 2.08-2.0 (m, 4H), 1.56-1.1 (m, 4H), 1.3 (m, 1H), 0.87 (d, 3H). MS (m/z): 459 [MH]⁺, 1Cl.
Preparation 53: 3-(4-Chloro-phenyl)-5-hydroxy-1-(4-methoxy-3-[2-piperidin-1-yl)-ethoxy]-phenyl)-pyrrolidin-2-one

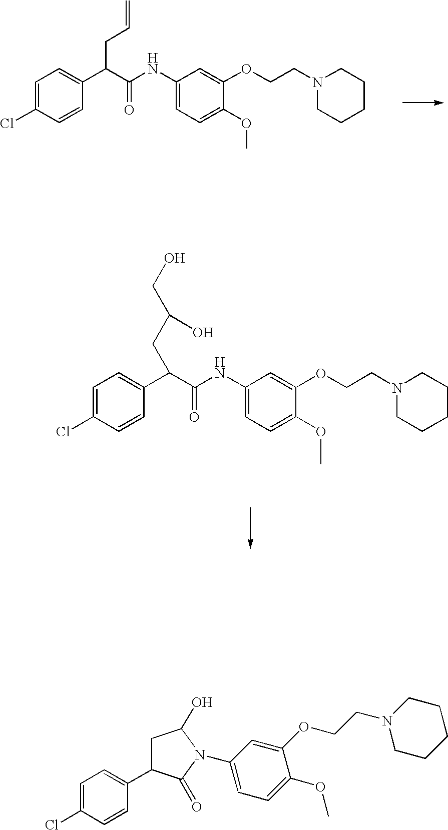

The title compound was prepared in an analogous manner to the compound of Preparation 18.

Two isomers A) and B) are present in 85/15 ratio.

NMR (¹H, DMSO-d6): δ 7.4-7.31 (d+d, 4H), 7.45-6.95 (m, 3H), 6.5-6.4 (d, 1H), 5.65 (m, 1H), 4.00 (t, 2H), 2.62 (t, 2H), 4.12-3.8 (m, 1H), 3.74 (s, 3H), 2.8-1.9 (m, 2H), 2.41 (m, 4H), 1.47 (m, 4H), 1.35 (m, 2H). MS (m/z): 445 [MH]⁺, 1Cl.
Preparation 54: 3-(2-Methoxy-5-nitro-phenoxymethyl)-piperidine-1-carboxylic Acid tert-butyl Ester

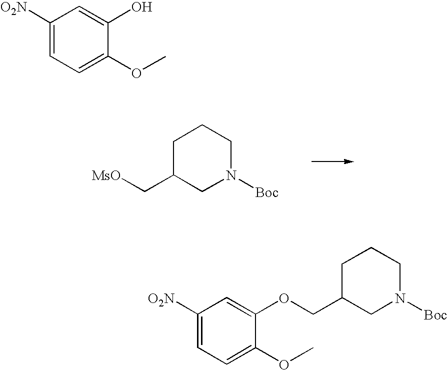

The product was prepared in an analogous manner to Preparation 6.

NMR (¹H, DMSO-d6): δ 7.91 (dd, 1H), 7.74 (d, 1H), 7.18 (d, 1H), 3.97 (d, 2H), 3.94 (bs, 2H), 3.90 (s, 3H), 2.90 (bs, 2H), 1.90 (m, 1H), 1.80 (bs, 1H), 1.60 (bs, 1H), 1.35 (bs, 2H), 1.35 (s, 9H). MS (m/z): 267 [MH-Boc]⁺.
Preparation 55: 3-(5-Amino-2-methoxy-phenoxymethyl)-piperidine-1-carboxylic Acid tert-butyl Ester

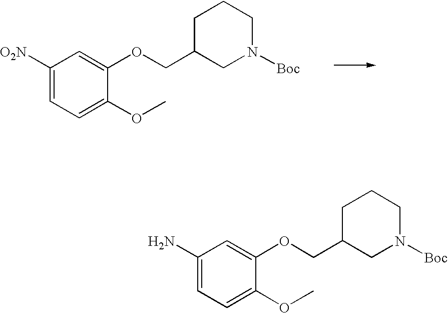

The product was prepared in an analogous manner to Preparation 44.

NMR (¹H, DMSO-d6): δ 6.64 (d, 1H), 6.25 (d, 1H), 6.07 (dd, 1H), 4.69 (bs, 2H), 3.73 (m, 2H), 3.67 (t, 1H), 3.60 (s, 3H), 2.8 (t, 1H), 1.9 (m, 1H), 1.80 (m, 1H), 1.6 (m, 1H), 1.4 (m, 3H), 1.38 (s, 9H), 1.25 (m, 1H). MS (m/z): 237 [MH-Boc]⁺.
Preparation 56: 3-{5-[2-(3,4-Dichloro-phenyl)-pent-4-enoylamino]-2-methoxy-phenoxymethyl}-piperidine-1-carboxylic Acid tert-butyl Ester

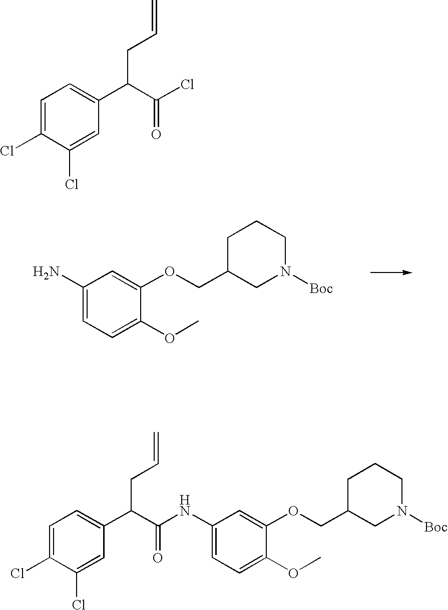

The product was prepared in an analogous manner to Preparation 14.

NMR (¹H, DMSO-d6): δ 9.96 (s, 1H), 7.55 (m, 2H), 7.32 (d, 1H), 7.22 (s, 1H), 7.01 (d, 1H), 6.82 (d, 1H), 5.66 (m, 1H), 5.03 (m, 1H), 4.94 (m, 1H), 3.9 (bs, 1H), 3.70 (m, 4H), 3.66 (s, 3H), 2.78 (m, 2H), 2.70 (m, 1H), 2.40 (m, 1), 1.83 (m, 1H), 1.75 (m, 1H), 1.57 (m, 1H), 1.31 (s, 9H), 1.25 (m, 2H), MS (m/z): 463 [MH-Boc]⁺, 2Cl.
Preparation 57: 3-{5-[3-(3,4-Dichloro-phenyl)-5-hydroxy-2-oxo-pyrrolidin-1-yl]-2-methoxy-phenoxymethyl}-piperidine-1-carboxylic Acid tert-butyl Ester

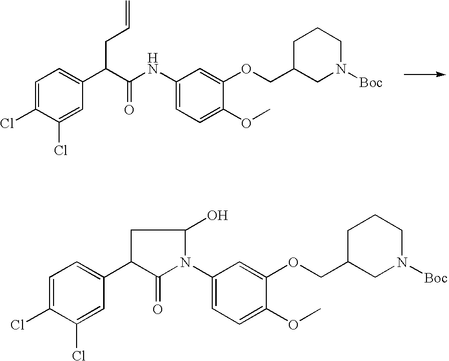

The product was prepared in an analogous manner to Example 15.

MS (m/z): 465 [M-Boc]⁺, 2Cl.
Preparation 58: N-(2-Hydroxy-4-nitro-phenyl)-acetamide

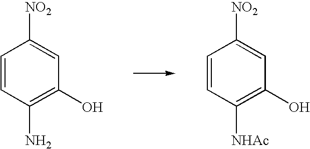

Procedure:

J. Med. Chem. 1998, 41, 13, 2308.

NMR (¹H, DMSO-d6): δ 10.99 (bs, 1H), 9.51 (s, 1H), 8.28 (d, 1H), 7.71 (dd, 1H), 7.66 (d, 1H), 2.2 (s, 3H).
Preparation 59: N-[4-Nitro-2-(2-piperidin-1-yl-ethoxy)-phenyl)-acetamide

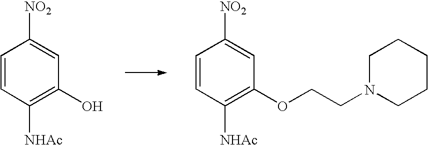

The product was prepared in an analogous manner to Preparation 6.

NMR (¹H, CDCl₃): δ 8.72 (bs, 1H), 8.55 (d, 1H), 7.91 (dd, 1H), 7.80 (d, 1H), 4.25 (t, 2H), 2.77 (t, 2H), 2.50 (m, 4H), 2.26 (s, 3H), 1.62 (m, 4H), 1.48 (m, 2H). MS (m/z): 308 [MH]⁺.
Preparation 60: N-[4-Amino-2-(2-piperidin-1-yl-ethoxy)-phenyl)-acetamide

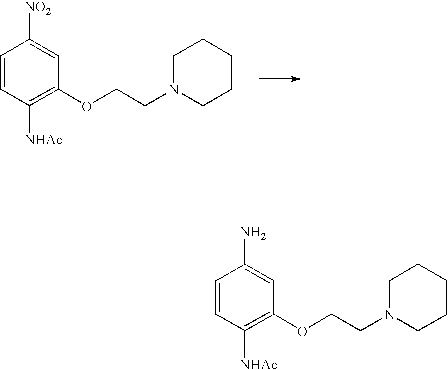

The product was prepared in an analogous manner to Preparation 10.

NMR (¹H, DMSO-d6): δ 8.63 (s, 1H), 7.23 (d, 1H), 6.25 (d, 1H), 6.07 (dd, 1H), 4.90 (s, 2H), 3.96 (t, 2H), 2.61 (t, 2H), 2.20 (s, 3H), 2.41 (m, 4H), 1.48 (m, 4H), 1.37 (m, 2H). MS (m/z): 278 [MH]⁺.
Preparation 61: 2-(3,4-Dichloro-phenyl)-pent-4-enoic Acid [4-acetylamino-3-(2-piperidin-1-yl-ethoxy)-phenyl]-amide

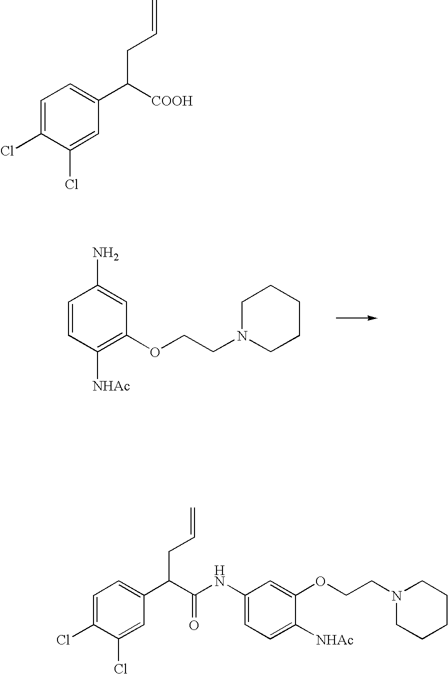

Procedure:

To a solution of 2-(3,4dichloro-phenyl)pent-4-enoic acid (210 mg, 1.08 mmol) in dry DMF (5 ml) were added HOBt (292 mg, 2 eq), EDC (310 mg, 1.5 eq) and TEA (0.376 ml, 2.5 eq). After 5 min. was added N-[4-Amino-2-(2-piperidin-1-yl-ethoxy)-phenyl)-acetamide (300 mg, 1 eq) dissolved in DMF (7 ml). After three days AcOEt was added, the organic phase was washed with 5% aq. NaHCO₃ and water, dried over Na₂SO₄ and concentrated in vacuo to give 398 mg of crude product, that was purified by flash chromatography (CH₂Cl₂/MeOH 9/1) to give 265 mg of the title compound (49% yield).

NMR (¹H, DMSO-d6): δ 10.1 (s, 1H), 8.92 (s, 1H), 7.77 (d, 1H), 7.61 (m, 2H), 7.40 (d, 1H), 7.38 (dd, 1H), 7.05 (dd, 1H), 5.73 (m, 1H), 5.03 (dd, 2H), 4.06 (t, 2H), 3.8 (t, 1H), 2.8 (m, 1H), 2.67 (t, 2H), 2.44 (m, 4H), 2.4 (m, 1H), 2.05 (s, 3H), 1.50 (m, 4H), 1.40 (m, 2H). MS (m/z): 504 [MH]⁺, 2Cl.
Preparation 62: N-[4-[3-(3,4-Dichloro-phenyl)-5-hydroxy-2-oxo-pyrrolidin-1-yl]-2-(2-piperidin-1-yl-ethoxy)-phenyl]-acetamide

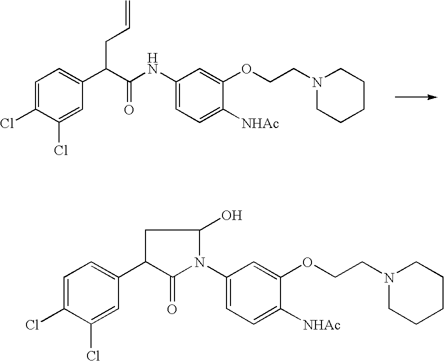

The product was prepared in an analogous manner to Example 15.

The product is a 60/40 mixture of a/b diastereoisomers.

NMR (¹H, DMSO-d6): δ 9.01 (s, 1H), 7.84 (d, 1H), 7.69 (d, 1Ha), 7.63 (m, 2H), 7.42 (dd, 1Hb), 7.40 (d, 1Ha), 7.32 (dd, 1Ha), 7.25 (d, 1Hb), 7.13 (dd, 1Ha), 7.02 (dd, 1Hb), 6.59 (d, 1Hb), 6.51 (d, 1Ha), 5.74, (m, 1Ha), 5.68 (m, 1Hb), 4.21 (m, 1Ha), 4.09 (t, 2H), 3.90 (m, 1Hb), 2.95 (m, 1Hb), 2.68 (m, 2H), 2.5 (m, 1Ha), 2.44 (m, 4H), 2.35 (m, 1Ha), 2.07 (s, 3H), 1.95 (m, 1Hb), 1.49 (m, 4H), 1.38 (m, 2H) . MS (m/z): 506 [MH]⁺, 2Cl.
Preparation 63: 2-(3,4-Dichloro-phenyl)-propionic Acid Methyl Ester

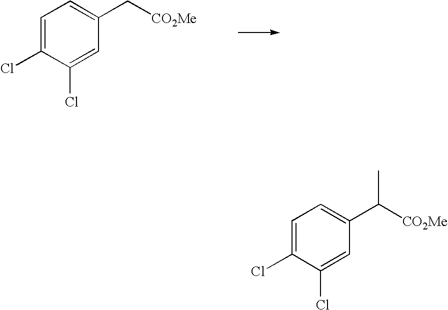

Procedure:

A solution of methyl-3,4-dichloro-phenylacetate (2 g, 9.13 mmol) in anh. THF (30 ml), under N₂, was treated with lithium bis(trimethylsilyl) amide (1 M solution in THF, 1.1 eq., 10 ml) at −78° C. for 30 minutes before methyl iodide (1.2 eq., 0.67 ml) was added. The mixture was stirred for 18 hours at room temperature, then quenched with water (20 ml) and extracted with ethyl acetate (2×15 ml). The organic phase was washed with brine (1×15 ml), dried over Na₂SO₄ and concentrated in vacuo to give 1.68 g of the title product as a orange oil (79%), that was used as a crude.

NMR (¹H, CDCl₃): δ 7.20 (m, 2H), 7.0 (dd, 1H), 3.50 (s, 3H), 3.50 (m, 1H), 1.3 (d, 3H).
Preparation 64: 2-(3,4-Dichloro-phenyl)-2-methyl-pent-4-enoic Acid Methyl Ester

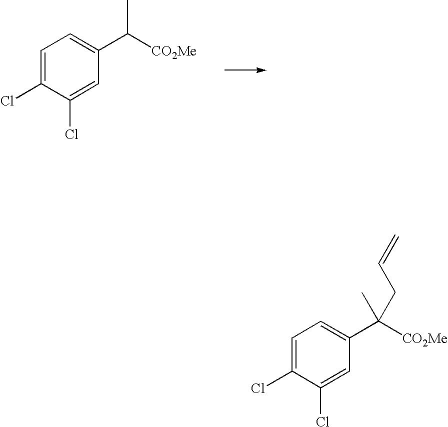

Procedure:

A solution of 2-(3,4-dichloro-phenyl)-propionic acid methyl ester (1.66 g, 7.12 mmol) in anh. THF (25 ml), under N₂, was treated with lithium bis(trimethylsilyl) amide (1M solution in THF, 1.1 eq., 7.8 ml) at −78° C. for 30 minutes before allyliodide (1.2 eq., 0.78 ml) was added. The mixture was stirred for 2 hours at room temperature, then quenched with water (20 ml) and extracted with ethyl acetate (2×15 ml). The organic phase was washed with brine (1×15 ml), dried over Na₂SO₄ and concentrated in vacuo. The crude product was purified by flash chromatography (silica gel, cHex/AcOEt 9/1) to give 1.70 g of the title product as a pale yellow oil (87%).

NMR (¹H, CDCl₃): δ 7.35 (m, 2H), 7.0 (dd, 1H), 5.40 (m, 1H), 4.95 (m, 2H), 3.50 (s, 3H), 2.6 (m, 1H), 2.4 (m, 1H), 1.2 (s, 3H).
Preparation 65: 2-(3,4-Dichloro-phenyl)-2-methyl-pent-4-enoic Acid

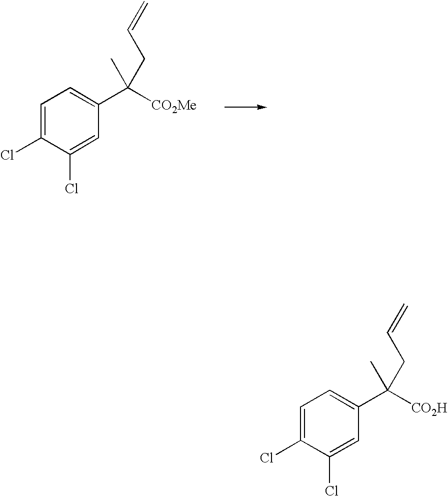

Procedure:

To a solution of 2-(3,4-dichloro-phenyl)-2-methyl-pent-4-enoic acid methyl ester (0.908 g, 3.32 mmol) in MeOH (33 ml), was added NaOH 1M (3 eq., 9.9 ml). The mixture was refluxed for 7 hours, then concentrated and washed with CH₂Cl₂. The aqueous phase was acidified to pH 3 with conc. HCl and extracted with CH₂Cl₂. The combined extracts were dried over Na₂SO₄ and concentrated in vacuo to give 806 mg of the title product as a colorless oil (94%), that was used as crude.

NMR (¹H, CDCl₃): δ 7.30 (m, 2H), 7.10 (dd, 1H), 5.40 (m, 1H), 4.95 (m, 2H), 2.60 (m, 1H), 2.45 (m, 1H), 1.50 (s, 3H).
Preparation 66: 2-(3,4-Dichloro-phenyl)-2-methyl-pent-4-enoic Acid {4-methoxy-3-[2-(4-methyl-piperidin-1-yl)-ethoxy]-phenyl}-amide

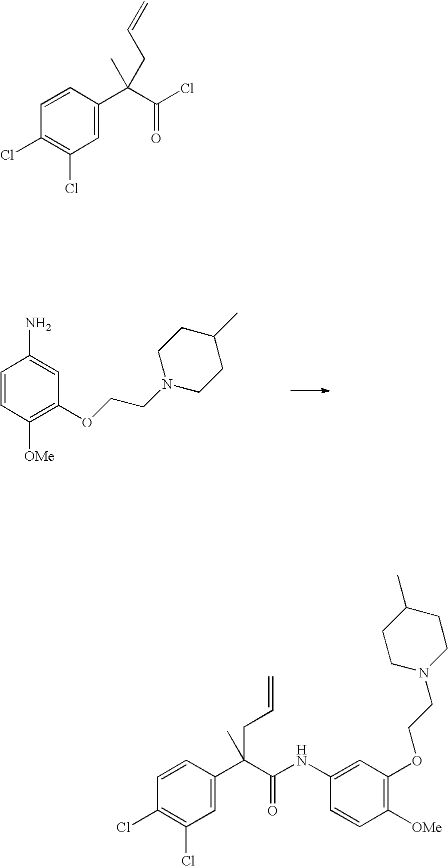

The product was prepared in an analogous manner to Preparation 14.

NMR (¹H, CDCl₃): δ 9.46 (bs, 1H), 7.30-6.80 (m, 5H), 6.59 (d, 1H), 5.35 (m, 1H), 4.94-4.80 (m, 2H), 4.04 (t, 2H), 3.60 (s, 3H), 3.34 (m, 2H), 3.10 (t, 2H), 2.70-2.28 (m, 4H), 1.64-1.29 (m, 1H), 1.43 (s, 3H), 1.25 (m, 4H), 0.72 (d, 3H). MS (m/z): 505 [MH]⁺, 2Cl.
Preparation 67: 3-(3,4-Dichloro-phenyl)-5-hydroxy-1-(4-methoxy-3-[2-(4-methyl piperidin-1-yl)-ethoxy]-phenyl}-3-methyl-pyrrolidin-2-one

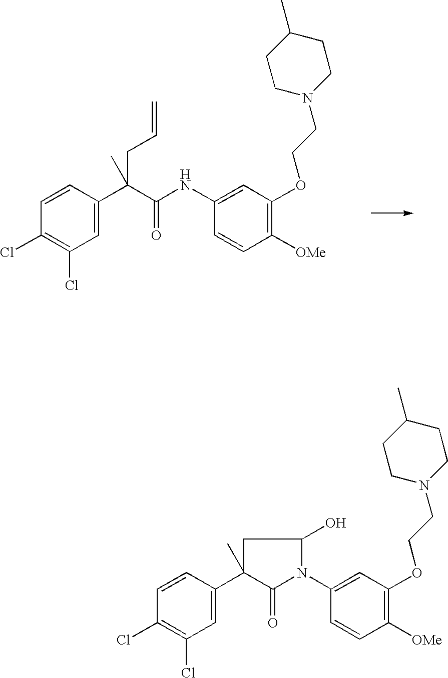

The product was prepared in an analogous manner to Example 15.

MS (m/z): 507 [MH]⁺, 2Cl.
Preparation 68: 2-(3-Chloro-phenyl)-pent-4-enoic Acid

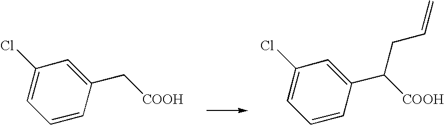

Procedure:

The reaction was carried out as described for Preparation 12 on 1.00 g of 3-chlorophenylacetic acid. The title compound (1.23 g) was recovered as an orange oil.

NMR (¹H, DMSO-d6): δ 12.50 (bs, 1H), 7.15-7.40 (m, 4H), 5.60-5.80 (m, 1H), 4.90-5.10 (m, 2H), 3.70 (t, 1H), 2.60-2.80 (m, 1H), 2.35-2.50 (m, 1H).
Preparation 69: 2-(3-Chloro-phenyl)-pent-4-enoic Acid [4-methoxy-3-(2-piperidin-1-yl-ethoxy)-phenyl]-amide

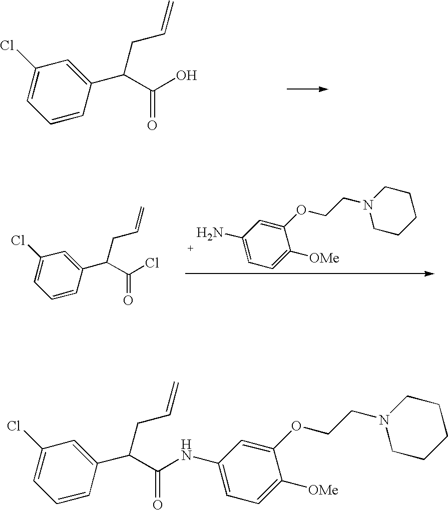

The reaction was carried out on 500 mg of 2-(3-chloro-phenyl)pent-4-enoic acid as described in Preparation 14. The title compound (760 mg) was obtained as a brown oil.

NMR (¹H, CDCl₃): δ 7.35 (bs, 1H), 7.18-7.28 (m, 3H), 7.10 (bs, 1H), 6.83 (dd, 1H), 6.75 (d, 1H), 5.64-5.80 (m, 1H), 4.95-5.12 (m, 2H), 4.10 (t, 2H), 3.78 (s, 3H), 3.45 (t, 1H), 2.85-3.00 (m, 2H), 2.77 (t, 2H), 2.40-2.60 (m, 5H), 1.52-1.65 (m, 4H), 1.36-1.47 (m, 2H). MS (m/z): 443 [MH]⁺, 1Cl.
Preparation 70: 3-(3-Chloro-phenyl)-5-hydroxy-1-[4-methoxy-3-(2-piperidin-1-yl-ethoxy)-phenyl]-pyrrolidin-2-one

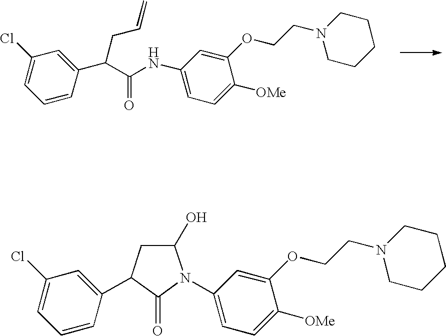

Procedure:

The reaction was carried out on 2-(3-chloro-phenyl)-pent-4-enoic acid [4-methoxy-3-(2-piperidin-1-yl-ethoxy)-phenyl]-amide (700 mg) as described for Example 15 to give the title compound (430 mg, mixture of two diastereomers as a brown foam.

MS (m/z): 445[MH]⁺, 1Cl.
Preparation 71: 2-(3-Trifluoromethyl-phenyl)-pent-4-enoic Acid

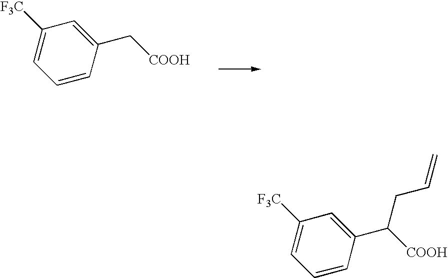

Procedure:

The reaction was carried out as described for Preparation 12 on 1.00 g of 3-trifluoromethylphenylacetic acid. The title compound (1.18 g) was recovered as a yellow gum.

NMR (¹H, DMSO-d6): δ 12.45 (bs, 1H), 7.50-7.70 (m, 4H), 5.60-5.75 (m, 1H), 4.90-5.00 (m, 2H), 3.80 (t, 1H), 2.65-2.80 (m, 1H), 2.40-2.50 (m, 1H).
Preparation 72: 2-(3-Trifluoromethyl-phenyl)-pent-4-enoic Acid [4-methoxy-3-(2-piperidin-1-yl-ethoxy)-phenyl]-amide

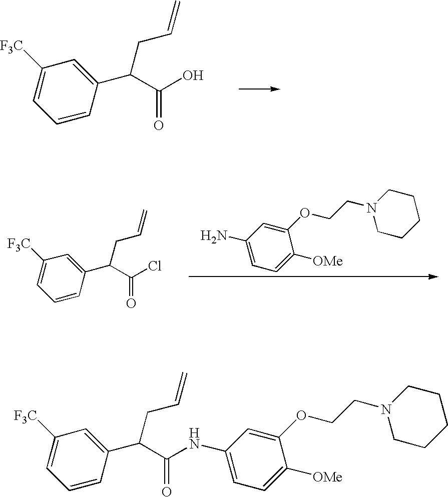

Procedure:

The reaction was carried out on 300 mg of 2-(3-trifluoromethyl-phenyl)pent-4-enoic acid as described in Preparation 14. The title compound (450 mg) was obtained as a white foam.

NMR (¹H, DMSO-d6): δ 10.03 (s, 1H), 7.71 (bs, 1 H), 7.70 (d, 1H), 7.66 (bs, 1H), 7.61 (d, 1H), 7.59 (t, 1H), 7.06 (dd, 1H), 6.86 (d, 1H), 5.72 (m, 1H), 5.09 (d, 1H), 4.99 (d, 1H), 3.96 (bs, 2H), 3.80 (t, 1H), 3.67 (s, 3H), 3.63 (bs, 2H), 2.8 (m, 1H), 2.6 (m, 1H), 2.41 (bs, 4H), 1.47 (bm, 4H), 1.40 (bs, 2H). MS (m/z): 477 [MH]⁺.
Preparation 73: 3-(3-Trifluoromethyl-phenyl)-5-hydroxy-1-[4-methoxy-3-(2-piperidin-1-yl-ethoxy)-phenyl]-pyrrolidin-2-one

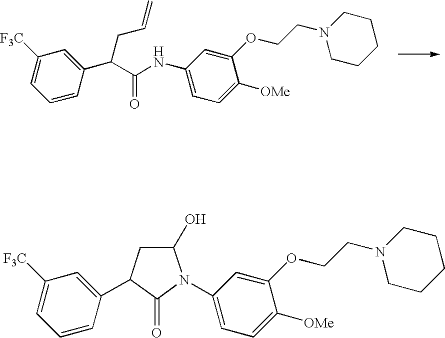

Procedure:

The reaction was carried out on 2-(3-trifluoromethyl-phenyl)-pent-4-enoic acid [4-methoxy-3-(2-piperidin-1-yl-ethoxy)-phenyl]-amide (450 mg) as described for Example 13 to give the title compound (285 mg, mixture of two diastereomers in 2.5/4 ratio) as a colourless oil.

NMR (¹H, CDCl₃) of the major product: δ 7.57-7.79 (m, 4H), 7.13 (dd, 1H), 7.01 (d, 1H), 6.99 (d, 1H), 6.46 (d, 1 H), 5.65 (m, 1H), 4.30 (t, 2H), 3.98 (t, 1H), 3.76 (s, 3H), 2.91 (m, 2H), 2.62 (bs, 4H), 2.50 (m, 2H), 2.39 (m, 2H), 1.58 (bm, 4H), 1.41 (bs, 2H).

MS (m/z): 479 [MH]⁺.
Preparation 74: 2-(4-Fluoro-phenyl)-pent-4-enoic Acid

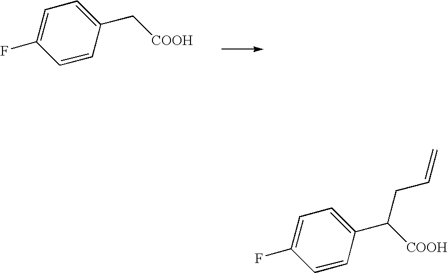

Procedure:

The reaction was carried out as described for Preparation 12 on 1.00 g of 4-fluoromethylphenylacetic acid. The title compound (1.26 g) was recovered as a white foam.

NMR (¹H, DMSO-d6): δ 12.40 (bs, 1H), 7.25-7.40 (m, 2H), 7.05-7.15 (m, 2H), 5.60-5.75 (m, 1H), 4.90-5.05 (m, 2H), 3.60 (t, 1H), 2.60-2.15 (m, 1H), 2.30-2.45 (m, 1H).
Preparation 75: 2-(4-Fluoro-phenyl)-pent-4-enoic Acid [4-methoxy-3-(2-piperidin-1-yl-ethoxy)-phenyl]-amide

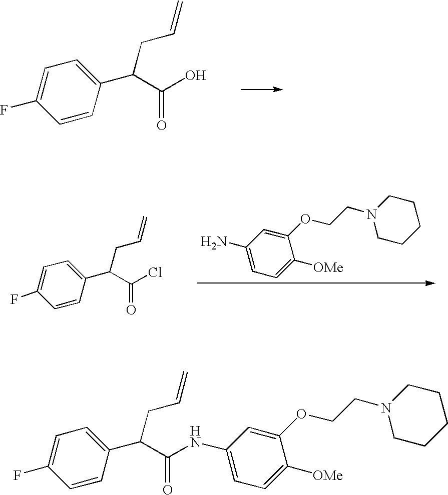

Procedure:

The reaction was carried out on 600 mg of 2-(4-fluoro-phenyl)-pent-4-enoic acid as described in Preparation 14. The title compound (880 mg) was obtained as an orange gum.

NMR (¹H, DMSO-d6): δ 9.94 (s, 1H), 7.41 (t, 2H), 7.28 (d, 1H), 7.14 (t, 2H), 7.05 (dd, 1H), 6.85 (d, 1H), 5.69 (m, 1H), 5.08 (d, 1H), 4.98 (d, 1H), 3.97 (t, 2H), 3.80 (t, 1H), 3.70 (t, 1H), 3.68 (s, 3H), 2.70 (m, 1H), 2.63 (t, 2H), 2.40 (bm, 4H), 1.48 (bm, 4H), 1.37 (bs, 2H). MS (m/z): 427 [MH]⁺.
Preparation 76: 3-(4-Fluoro-phenyl)-5-hydroxy-1-[4-methoxy-3-(2-piperidin-1-yl-ethoxy)-phenyl]-pyrrolidin-2-one

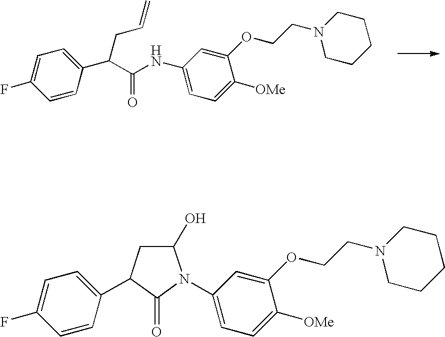

Procedure:

The reaction was carried out on 2-(4-fluoromethyl-phenyl)-pent-4-enoic acid [4-methoxy-3-(2-piperidin-1-yl-ethoxy)-phenyl]-amide (750 mg) as described for Example 13 to give the title compound (570 mg, mixture of two diastereomers) as a brown foam.

MS (m/z): 429 [MH]⁺.

### EXAMPLE 1

### 3-(3,4-Dichloro-phenyl)-3-hydroxy-1-[4-methoxy-3-(2-piperidin-1-yl-ethoxy)-phenyl]-pyrrolidin-2-one

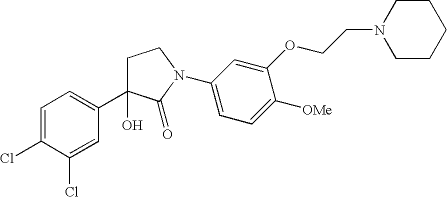

To a solution of methansulfonic acid 2-(5-[3-(3,4-dichloro-phenyl)-2-oxo-pyrrolidin-1-yl]-2-methoxy-phenoxy)-ethyl ether (103 mg, 0.216 mmol) in DMF (5 ml) was added piperidine (1 eq, 22 μl), Nal (cat.) and K₂CO₃ (1 eq, 15.5 μl). The reaction mixture was heated at 60° C. for 5 hours under atm N₂ and then 18 hours at room temperature in the presence of air. The reaction mixture was diluted with water (20 ml) and extracted with AcOEt (3×25 ml). The combined organic extracts were dried over Na₂SO₄, filtered and concentrated in vacuo The crude product was purified by flash chromatography (silica gel, CH₂Cl₂/CH₃OH 18/2) to give 1.6 mg of title product as a yellow oil (3.8%).

NMR (¹H, DMSO-d6): δ 7.68 (d, 1H), 7.62 (d, 1H), 7.48 (d, 1H), 7.40 (dd, 1H), 7.12 (dd, 1H), 6.97 (d, 1H), 6.48 (s, 1H), 4.02 (t, 2H), 3.91-3.80 (m, 2H), 3.74 (s, 3H). 2.64 (m, 2H), 2.51-2.3 (m, 2H), 2.42 (m, 4H), 1.47-1.35 (m, 2H). MS (m/z): 479 [MH]⁺+2Cl

### EXAMPLE 2

### 3-(3,4-Dichloro-phenyl)-3-hydroxy-1-[4-methoxy-3-(2-morpholin-4-yl-ethoxy)-phenyl]-pyrrolidin-2-one

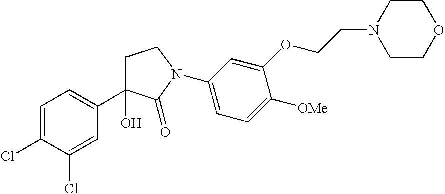

The product was prepared in an analogous manner as for Example 1.

NMR (¹H, DMSO-d6): δ 7.67 (d, 1H), 7.62 (d, 1H), 7.48 (d, 1H), 7.40 (dd, 1H), 7.12 (dd, 1H), 6.98 (d, 1H), 6.48 (s, 1H), 4.05 (t, 2H), 3.90-3.80 (m, 2H), 3.74 (s, 3H), 3.74 (s, 3H), 3.56 (m, 4H), 2.68 (m, 2H), 2.49-2.34 (m, 2H), 2.48 (m, 4H). MS (m/z): 481 [MH]⁺+2Cl

### EXAMPLE 3

### 3-(3,4-Dichloro-phenyl)-1-[4-methoxy-3-(2-piperidin-1-yl-ethoxy)-phenyl]-pyrrolidin-2-one

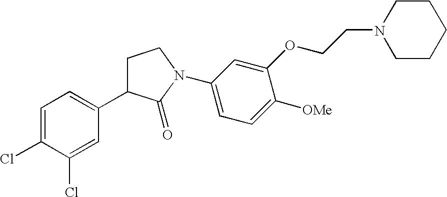

To a solution of crude methansulfonic acid 2-(5-[3-(3,4-dichloro-phenyl)-2-oxo-pyrrolidin-1-yl]-2-methoxy-phenoxy)-ethyl ether (0.09 mmol) in DMF (2 ml) was added piperidine (1 eq, 9 μl), Nal (cat.) and DIPEA (1 eq, 15.5 μl). The reaction mixture was heated at 100° C. for 3 hours. The reaction mixture was diluted with water (20 ml) and extracted with AcOEt (3×25 ml). The combined organic extracts were dried over Na₂SO₄, filtered and concentrated in vacuo The crude product was purified by flash chromatography (silica gel, CH₂Cl₂/CH3OH 18/2) to give 1.6 mg of the title product as a pale yellow solid (3.8%).

NMR (¹H, CDCl₃): δ 0.7.56 (d, 1H), 7.45-7.43 (m, 2H), 7.18 (dd, 1H), 6.96 (dd, 1H), 6.86 (d, 1H), 4.19 (t, 2H), 3.95-3.8 (m, 4H+2H), 2.65-2.57 (m, 1H+4H), 2.25 (m, 1H), 1.9-1.5 (m, 6H). MS (m/z): 463.4 [MH]⁺+2Cl

### EXAMPLE 4

### 1-[4-Chloro-3-(2-piperidin-1-yl-ethoxy)-phenyl]-3-(3,4-dichloro-phenyl)-pyrrolidin-2-one

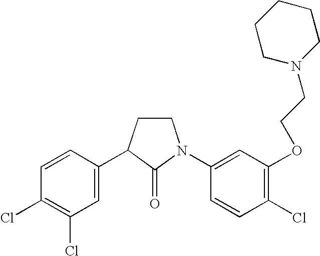

The title compound was prepared in an analogous manner to the compound of Example 3.

NMR (¹H, CDCl₃): δ 7.72 (d, 1H), 7.45 (d, 1H), 7.42 (d, 1H), 7.33 (d, 1H), 7,18 (dd, 1H), 6.90 (dd, 1H), 4.19 (t, 2H), 3.91 (dd, 2H), 3.84 (t, 1H), 2.86 (t, 2H), 2.65 (m, 1H), 2.57 (m, 4H), 2.3 (m, 1H) 1.6-1.4 (m, 6H). MS (m/z): 467 [MH]⁺, 3Cl.

### EXAMPLE 5

### 1-[4-Chloro-3-(2-piperidin-1-yl-ethoxy)-phenyl]-3-(3,4-dichloro-phenyl)-3-hydroxy-pyrrolidin-2-one

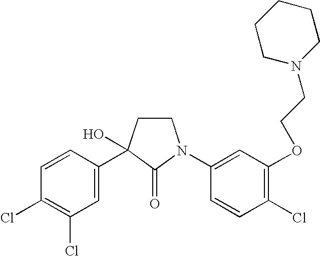

1-[4-Chloro-3-(2-piperidin-1-yl-ethoxy)-phenyl]-3-(3,4-dichloro-phenyl)-pyrrolidin-2-one (26 mg, 0.055 mmol) was dissolved in DMF (1 ml), tBuOK (9 mg, 1.3 eq) was added and the mixture was stirred at room temperature in the presence of air for 30 min. A saturated solution of NH₄Cl was added and extracted with CH₂Cl₂. The extracts were dried over Na₂SO₄ and concentrated in vacuo. The residue was purified by flash chromathography (silica gel, CH₂Cl₂/MeOH 9/1), to give 13 mg of the title product (49% yield).

NMR (¹H, CDCl₃): δ 7.70 (d, 1H), 7.63 (m, 2H), 7.43 (d, 1H), 7.42 (dd, 1H), 7.28 (dd, 1H), 6.57 (s, 1H), 4.14 (t, 2H), 3.98 (m, 1H), 3.85 (m, 1H), 2.69 (m, 2H), 2.53 (m, 1H), 2.45 (m, 4H), 2.35 (m, 1H), 1.47-1.35 (m, 6H). MS (m/z): 483 [MH]⁺, 3Cl.

### EXAMPLE 6

### 3-(3,4-Dichloro-phenyl)-1-(4-methoxy-3-[2-(4-methyl-piperidin-1-yl)-ethoxy]-phenyl)-pyrrolidin-2-one

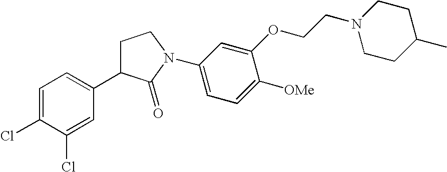

The title compound was prepared in an analogous manner to the compound of Example 3.

NMR (¹H, CDCl₃): δ 0.7.57 (d, 1H), 7.44 (d+d, 1H+1H), 7.19 (dd, 1H), 6.96 (dd, 1H), 6.86 (d, 1H), 4.17 (bt, 2H), 3.90 (m, 2H), 3.83 (t, 1H), 3.86 (s, 3H), 3.00 (m, 2H). 2.84 (bt, 2H), 2.66 (m, 1H), 2.28 (m, 1H), 2.10 (m, 2H), 1.6-1.2 (m, 5H), 0.93 (d, 3H). MS (m/z): 477 [MH]⁺+2Cl

### EXAMPLE 7

### 3-(3,4-Dichloro-phenyl)-1 (4methoxy-3-(2-piperidin-1-yl-ethoxy)-phenyl]-1,5-dihydro-pyrrol-2-one

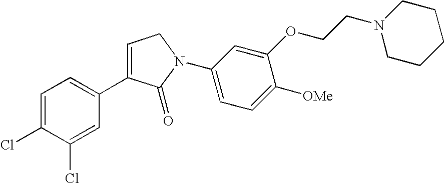

3-(3,4-Dichloro-phenyl)-3-hydroxy-1-[4-methoxy-3-(2-piperidin-1-yl-ethoxy)-phenyl]-pyrrolidin-2-one (17.2 mg, 0.035 mmol) was dissolved in concentrated hydrochloric acid (1.7 ml). The reaction was heated at 40° C. for 3 hours, and then concentrated to dryness in vacuo. A solution of saturated NaHCO₃ was added until pH>7 and the product was extracted with ethylacetate (2×10 ml). The combined extracts were dried over anhydrous Na₂SO₄, filtered and concentrated to dryness in vacuo. The crude product was purified by flash chromatography (silica gel, AcOEt/CH₃OH 4/6) to give 11.8 mg of title product as a brown oil (55.2%).

NMR (¹H, CDCl₃): 8.05 (d, 1H), 7.78 (d, 1H), 7.63 (d, 1H), 7.48 (d, 1H), 7.31 (t, 1H), 7.04 (dd, 1H), 6.88 (d, 1H), 4.45 (d, 2H), 4.20 (t, 2H), 3.87 (s, 3H). 2.85 (t, 2H), 2.54 (m, 4H), 1.62 (m, 4H), 1.45 (m, 2H). MS (m/z): 461.1 [MH]⁺+2Cl

### EXAMPLE 8

### 3-(3,4-Dichloro-phenyl)-1-[4-methoxy-3-(2-morpholin-4-yl-ethoxy)-phenyl]-1,5-dihydro-pyrrol-2-one

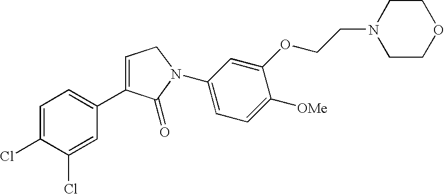

The product was prepared in an analogous manner to Example 7.

NMR (¹H, CDCl₃): δ 0.8.04 (d, 1H), 7.77 (dd, 1H), 7.72 (d, 1H), 7.48 (d, 1H), 7.31 (t, 1H), 6.96 (dd, 1H), 6.88 (d, 1H), 4.45 (d, 2H), 4.21 (t, 2H), 3.86 (s, 3H), 3.74 (m, 4H), 2.87 (t, 2H), 2.60 (m, 4H). MS (m/z): 463 [MH]⁺+2Cl

### EXAMPLE 9

### 1-[4-Chloro-3-(2-piperidin-1-yl-ethoxy)-phenyl]-3-(3,4-dichloro-phenyl)-1,5-dihydro-pyrrol-2-one

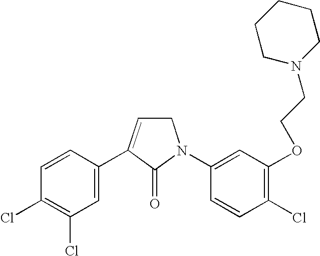

1-[4-Chloro-3-(2-piperidin-1-yl-ethoxy)-phenyl]-3-(3,4-dichloro-phenyl)-5hydroxy-pyrrolidin-2-one (51 mg, 0.1 mmol) was dissolved in TFA (1 ml). The reaction mixture was stirred at room temperature for 6 hours, then concentrated in vacuo. A saturated solution of NaHCO₃ was added and the mixture was extracted with ethyl acetate, dried over Na₂SO₄ and concentrated in vacuo. The residue was purified by flash chromathography over silica gel (CH₂Cl₂/MeOH 95/5) to give 29 mg of the title product as a colorless oil (yield: 60%).

NMR (¹H, CDCl₃): δ 8.0 (d, 1H), 7.83 (d, 1H), 7.73 (dd, 1H), 7.46 (d, 1H), 7,32 (d, 1H), 7.30 (m, 1H), 6.96 (dd, 1H), 4.43 (d, 2H), 4.21 (t, 2H), 2.85 (t, 2H), 2.56 (m, 4H), 1.5-1.4 (m, 6H). MS (m/z): 465 [MH]⁺, 3Cl.

### EXAMPLE 10

### 3-(3,4-Dichloro-phenyl)-1-[4-methoxy-3-(2-pyrrolidin-1-yl-ethoxy)-phenyl]-1,5-dihydro-pyrrol-2-one

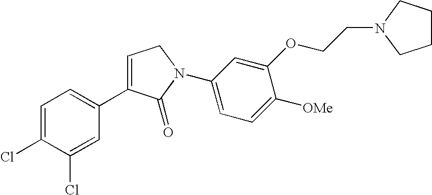

The title product was prepared in an analogous manner to Example 7.

NMR (¹H, CDCl₃): δ 0.8.06 (d, 1H), 7.78 (dd, 1H), 7.64 (d, 1H), 7.49 (d, 1H), 7.32 (t, 1H), 7.05 (dd, 1H), 6.89 (d, 1H), 4.45 (d, 2H), 4.22 (t, 2H), 3.87 (s, 3H), 2.99 (t, 2H), 2.68 (m, 4H), 1.83-1.77 (m, 4H). MS (m/z): 447 [MH]⁺+2Cl

### EXAMPLE 11

### 3-(3-Fluoro-phenyl)-1-[4-methoxy-3-(2-piperidin-1-yl-ethoxy)-phenyl]-1,5-dihydro-pyrrol-2-one

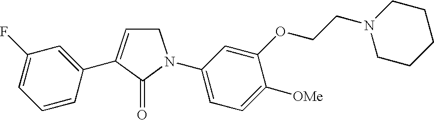

The title compound was prepared in an analogous manner to the compound of Example 3.

NMR (¹H, CDCl₃): δ 7.55-7.70 (m, 3H), 7.25-7.40 (m, 2H), 6.95-7.10 (m, 2H), 6.85 (d, 1H), 4.40 (d, 2H), 4.10-4.20 (m, 2H), 3.85 (s, 3H), 2.85 (t, 2H), 2.45-2.60 (m, 4H), 1.55-1.70 (m, 4H), 1.35-1.50 (m, 2H). MS (m/z): 411[MH]⁺.

### EXAMPLE 12

### 3-(3,4-Dichloro-phenyl)-1-(-3-[2-(4,4-difluoro-piperidin-1-yl)-ethoxy]-4-methoxy-phenyl)-1,5-dihydro-pyrrol-2-one

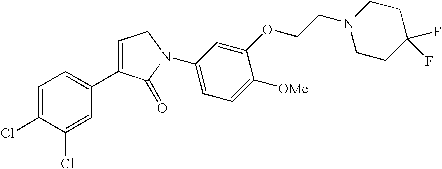

The title compound was prepared in an analogous manner to the compound of Example 3.

NMR (¹H, CDCl₃): δ 8.06 (d, 1H), 7.78 (dd, 1H), 7.76 (d, 1H), 7.50 (d, 1H), 7.33 (t, 1H), 6.99 (bd, 1H), 6.90 (d, 1H), 4.44 (d, 2H), 4.22 (m, 2H), 3.87 (s, 3H), 2.95 (m, 2H), 2.75 (m, 4H), 2.05 (m, 4H). MS (m/z): 497.3 [MH]⁺+2Cl

### EXAMPLE 13

### 3-(3-Fluoro-phenyl)-5-hydroxy-1-[4-methoxy-3-(2-piperidin-1-yl-ethoxy)-phenyl]-pyrrolidin-2-one

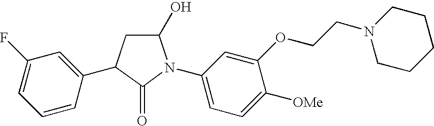

To a solution of 2-(3-fluoro-phenyl)-pent-4-enoic acid [4methoxy-3-(2-piperidin-1-yl-ethoxy)-phenyl]-amide (85 mg, 0.2 mmol) in THF/H₂O (5/1, 4 mL) was added OsO₄ (4% wt in water, 0.02 mmol) and NalO₄ (86 mg, 0.4 mmol). After 18 hours the suspension was diluted with water and the aqueous phase was extracted with EtOAc (2×). The combined organic phases were dried over Na₂SO₄, filtered and concentrated to dryness in vacuo. The crude was purified by flash chromatography (CH₂Cl₂/MeOH/NH₄OH 95/5/0.5) to give 65 mg of title product as a brown oil. (y=75%) MS (m/z): 29[MH]⁺.

### EXAMPLE 14

### 1-[4-Chloro-3-(2-piperidin-1-yl-ethoxy)-phenyl]-3-(3,4-dichloro-phenyl)-5-hydroxy-pyrrolidin-2-one

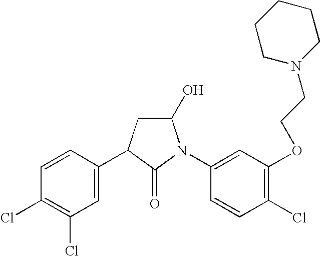

The title compound was prepared in an analogous manner to the compound of Example 13. Two isomers A) and B) are present in 1:2 ratio.

NMR (¹H, DMSO-d6):

A) δ 7.70-7.10 (m, 6H), 6.58 (d, 1H), 4.25 (t, 1H), 5.74 (m, 1H), 4.13 (bs, 2H), 2.63 (bs, 2H), 2.3-2.5 (m, 2H), 1.36-1.50 (bs, 10H).

B) δ 7.70-7.10 (m, 6H), 6.64 (d, 1H), 5.81 (m, 1H), 4.13 (bs, 2H), 3.93 (m, 1H), 2.63 (bs, 2H), 2.92 (m, 1H), 1.93 (m, 1H), 1.36-1.50 (bs, 10H).

MS (m/z): 454 [MH] +2Cl.

### EXAMPLE 15

### 3-3,4-Dichloro-phenyl)-1-(-3-[2-(4,4-difluoro-piperidin-1-yl)-ethoxy]-4-methoxy-phenyl)-5-hydroxy-pyrrolidin-2-one

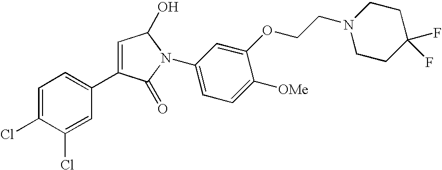

The title compound was prepared in an analogous manner to the compound of Example 13.

NMR (¹H, DMSO-d6): δ 7.7-7.6 (d, 1H), 7.6 (d, 1H), 7.4-7.3 (dd, 1H), 7.25-7.12 (d, 1H), 7.10-6.95 (dd, 1H), 6.9 (d, 1H), 6.55-6.43 (d, 1H), 5.66-5.60 (m, 1H), 4.03 (t, 2H), 4.2-3.85 (m, 1H), 3.74 (s, 3H), 2.77 (t, 2H), 2.9-2.43 (m, 2H), 2.62 (m, 4H), 1.94 (m, 4H). MS (m/z): 515 [MH]⁺+2Cl

### EXAMPLE 16

### 3-(3-Fluoro-phenyl)-1-[4-methoxy-3-(2-piperidin-1-yl-ethoxy)-phenyl]-pyrrolidin-2-one

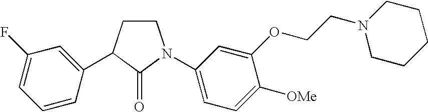

The title compound was prepared in an analogous manner to the compound of Example 3.

NMR (¹H, CDCl₃): δ 7.56 (d, 1H), 7.30 (m, 1H), 7.08 (d, 1H), 7.02 (m, 1 H), 6.95 (m, 1H), 6.93 (dd, 1H), 6.83 (d, 1H), 4.16 (t, 2H), 3.9-3.8 (m, 3H), 3.82 (s, 3H), 2.84 (t, 2H), 2.64 (bs, 4H), 2.62 (m, 1H), 2.24 (m, 1H), 1.60 (m, 4H), 1.43 (m, 2H). MS (m/z): 413[MH]⁺.

### EXAMPLE 17

### 3-(3,4-Dichloro-phenyl)-1-[4-methoxy-3-(2-pyrrolidin-1-yl-ethoxy)-phenyl]-pyrrolidin-2-one Hydrochloride Salt

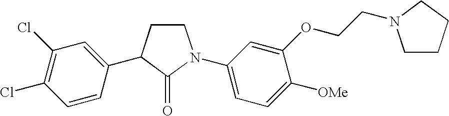

To 3-(3,4-dichloro-phenyl)-5-hydroxy-1-[4-methoxy-3-(2-pyrrolidin-1-yl-ethoxy)phenyl]-pyrrolidin-2-one (17 mg) in DCM (dry, 0.3 ml) and Et₃SiH (0.03 ml) at 0° C. was added dropwise a solution of trifluoroacetic acid (0.028 ml) in DCM (dry, 0.3 ml). The mixture was allowed to warm to 25° C. After 8 h volatiles were removed in vacuo and the residue submitted to column chromatography (silica gel, CH₂Cl₂/MeOH /NH₃) to give, after conversion to the hydrochloride salt by treatment with excess HCl (1 M in Et₂O) followed by evaporation in vacuo and trituraton of the residue with Et₂O, the title compound (8 mg) as a slightly yellow film.

NMR (¹H, CD₃OD): δ 7.60 (d, 1H), 7.54-7.5 (m, 2H), 7.30 (dd, 1H), 7.12 (dd, 1H), 7.08 (d, 1H), 4.35 (t, 2H), 4.02-3.92 (m, 3H), 3.89 (s, 3H), 3.82-3.76 (m, m, 2H), 3.65 (t, 2H), 3.28-3.20 (m, 2H), 2.71-2.63 (m, 1H), 2.32-2.16 (m, 3H), 2.11-2.01 (m, 2H). MS (m/z): 449 [MH]⁺, 2Cl.

### EXAMPLE 18

### 3-(3,4-Dichloro-phenyl)-1-[4-methoxy-3-(2-piperidin-1-yl-ethoxy)-phenyl]-3-methyl-pyrrolidin-2-one Hydrochloride Salt

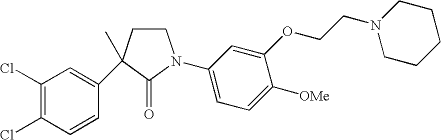

The title compound (racemic, colourless foam. MS (m/z): 477 [MH]⁺, 2Cl.) was prepared from 2-(3,4-dichloro-phenyl)-2-methyl-pent-4-enoic acid and 4-methoxy-3-(2-piperidin-1-yl-ethoxy)-phenylamine in an analogous manner to the one described for Preparation 28 and Example 15.

Separation of the enantiomers was achieved by preparative HPLC using a chiral stationary phase followed by salt formation as described for Example 19 to give Enantiomer 1 (slightly yellow solid) and Enantiomer 2 (slightly yellow solid) of the title compound.

Enantiomer 1: NMR (¹H, CD₃OD): δ 7.60 (d, 1H), 7.56 (d, 1H), 7.48 (d, 1H), 7.36 (dd, 1H), 7.10-7.02 (m, 2H), 4.34 (t, 2H), 3.92-3.82 (m, 4H), 3.80-3.74 (m, 1H), 3.68 (bd, 2H), 3.53 (t, 2H), 3.06 (bt, 2H), 2.57-2.50 (m, 1H), 2.36-2.28 (m, 1H), 2.00-1.93 (m, 2H), 1.88-1.73 (m, 3H), 1.60-1.45 (m, 4H).

Enantiomer 2: NMR (¹H, CD₃OD): Essentially identical to the data for Enantiomer 1.

### EXAMPLE 19

### 3-(3-Chloro-phenyl)-1-{4-methoxy-3-[2-(4-methyl-piperidin-1-yl)-ethoxy]-phenyl}-1,5-dihydro-pyrrol-2-one Hydrochloride Salt

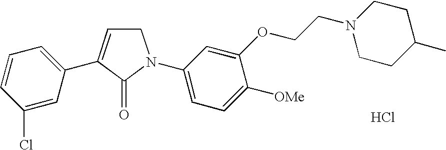

The title compound was prepared, in an analogous manner to the compound of Example 15 (HCl salt formation), in 44 mg yield as yellow solid (y=62%) from 2-(3-chloro-phenyl)-5-hydroxy-1-{4-methoxy-3-[2-(4-methylpiperidin)-1-yl-ethoxy]-phenyl}-pyrrolidin-2-one (90 mg)

NMR (¹H, DMSO): δ 9.85 (bs, 1H), 8.11 (s, 1H), 7.94 (d, 1H), 7.90 (t, 1H), 7.68 (d, 1H), 7.45 (m, 2H), 7.28 (dd, 1H), 7.06 (d, 1H), 4.62 (d, 2H), 4.37 (t, 2H), 3.78 (s, 3H), 3.60 (m, 2H), 3.40 (m, 2H), 3.00 (m, 2H), 1.82 (m, 2H), 1.60 (m, 1H), 1.45 (m, 2H), 0.90 (d, 3H).

MS (m/z): 441 [MH]⁺, 1Cl. Mp=99-101° C.

### EXAMPLE 20

### 3-(3,4-Dichloro-phenyl)-1-[4-methoxy-3-(2-piperidin-1-yl-ethoxy)-phenyl]-3,4-dihydro-1H-pyridin-2-one

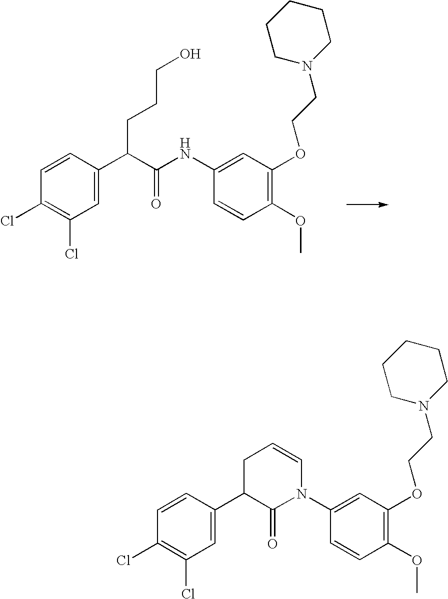

Procedure:

To a solution of 2-(3,4-dichloro-phenyl)-5-hydroxy pentanoic acid [4-methoxy-3-(2-piperidin-1-yl-ethoxy)-phenyl]-amide (330 mg, 0.66 mmol) in anh. CH₂Cl₂ (6 ml), under N₂, Dess-Martin periodane (1.3 eq., 365 mg), was added. The reaction was stirred at room temperature for 2 h. CH₂Cl₂ was added (10 ml) and the organic phase was washed with NaHCO₃ sat.(10 ml), the aqueous phase was extracted with CH₂Cl₂ (3×10 ml), the organic phases were dried over Na₂SO₄ and concentrated in vacuo to give a white foam. To a solution of the white foam in anh. CH₂Cl₂ (6 ml) ), under N₂, at 0° C., triethylamine (2.2 eq., 0.202 ml) and mesyl chloride (1.4 eq., 0.071 ml) were added. The reaction was stirred at room temperature for 16 h. CH₂Cl₂ was added (10 ml) and the organic phase was washed with NaHCO₃ sat. (10 ml), dried over Na₂SO₄ and concentrated in vacuo to obtain 23 mg of the title product as white foam.

NMR (¹H, DMSO-d6): δ 7.6 (s, 1H), 7.58 (d, 1H), 7.33 (dd, 1H), 6.98 (d, 1H), 6.90 (bs, 1H), 6.80 (dd, 1H), 6.43 (d, 1H), 5.35 (m, 1H), 4.01 (t, 2H), 4.00 (m, 1H), 3.76 (s, 1H), 2.70 (m, 1H), 2.60 (m, 1H), 2.58 (t, 2H), 2.48 (bs, 2H), 1.50 (bs, 4H), 1.39 (bs, 2H). MS (m/z): 475 [MH]⁺, 2Cl.

### EXAMPLE 21

### 3-(3,4-Dichloro-phenyl)-1-[4methoxy-3-(2-piperidin-1-yl-ethoxy)-phenyl]-piperidin-2-one Hydrochloride Salt

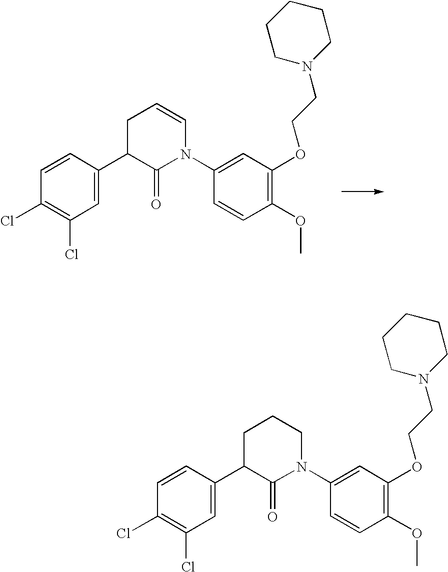

Procedure:

To a solution of 3-(3,4-dichloro-phenyl)-1-[4-methoxy-3-(2-piperidin-1-yl-ethoxy)-phenyl]-3,4-dihydro-1H-pyridin-2-one (50 mg, 0.105 mmol) in anh. CH₂Cl₂ (1 ml), under N₂, was added triethyl silane (3 eq., 0.05 ml). The reaction was stirred at room temperature for 1 h, then trifluoroacetic acid (6 eq., 0.05 ml) was added. The reaction was stirred at room temperature for 16 h. The reaction was concentrated in vacuo. The crude product was purified by flash chromatography (silica gel, CH₂Cl₂/MeOH from 24/1 to 4/1) to obtain 10 mg of desired compound as the free base.

NMR (¹H, CDCl₃): δ 7.36 (d, 1H), 7.34 (d, 1H), 7.10 (dd, 1H), 6.90 (bs, 3H), 4.37 (s, 2H), 3.80 (s, 3H), 3.8-3.6 (m, 5H), 3.42 (s, 2H), 2.83 (t, 2H), 2.30 (m, 1H), 2.1-1.8 (m, 7H), 1.35 (m, 2H). MS (m/z): 477 [MH]⁺, 2Cl.

From this material the hydrochloride salt was obtained as described for Example 17.

MS (m/z): 477 [MH]⁺, 2Cl.

### EXAMPLE 22

### 3-(3,4-Dichloro-phenyl)-1-(4-methoxy-3-[2-(4-methyl-piperidin-1-yl)-ethoxy]-phenyl)-3,4-dihydro-pyrrol-2-one Hydrochloride Salt

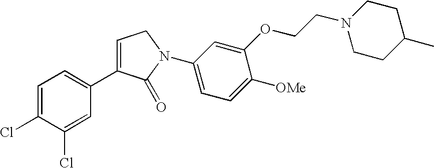

The title compound was prepared in an analogous manner to the one described for the compound of Example 9 and 17 (HCl salt formation).

NMR (¹H, DMSO-d₆): δ 0.9.8+9.5 (2bs, 1H), 8.30 (d, 1H), 7.97 (dd, 1H), 7.94 (t, 1H), 7.68 (d, 1H), 7.65 (d, 1H), 7.23 (dd, 1H), 7.03 (d, 1H), 4.60 (d, 2H), 4.32 (t, 2H), 3.75 (s, 3H), 3.56 (d, 2H), 3.45 (m, 2H), 3.01 (q, 2H), 1.78 (d, 2H), 1.57 (bm, 1H), 1.38 (q, 2H), 0.89 (d, 3H). MS (m/z): 477 [MH]⁺, 2Cl

### EXAMPLE 23

### 3-(3,4-Dichloro-phenyl)-1-(4methoxy-3-[2-(4-methyl-piperidin-1yl)-ethoxy]-phenyl)-4-methyl-1,5-dihydro-pyrrol-2-one Hydrochloride Salt

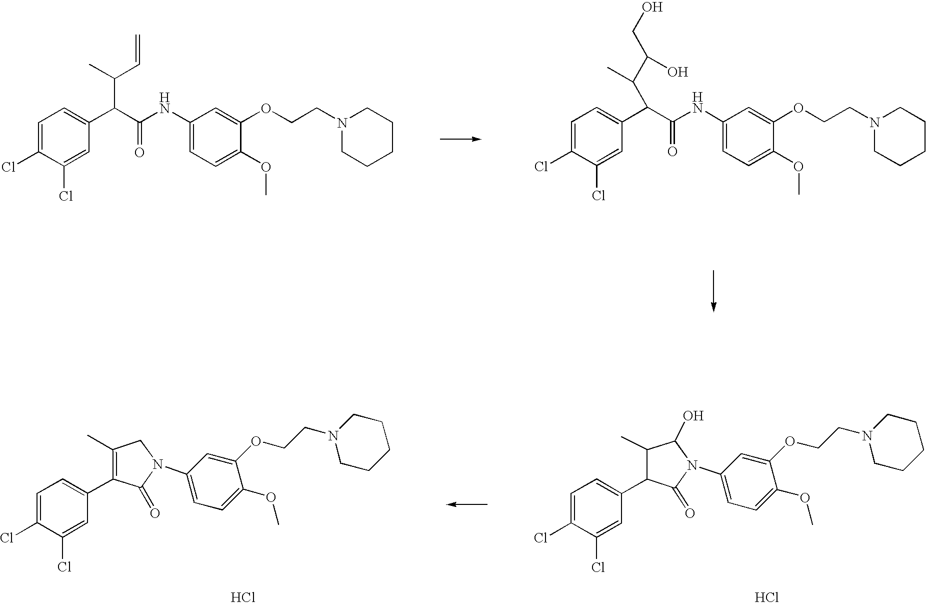

The title compound was prepared in an analogous manner to the one described for the compound of Example 13 followed by Example 9 and 17 (HCl salt formation).

NMR (¹H, CD₃OD): δ 7.74 (d, 1H), 7.63 (d, 1H), 7.71 (d, 1H), 7.44 (dd, 1H), 7.18 (dd, 1H), 7.09 (d, 1H), 4.53 (s, 2H), 4.34 (t, 2H), 3.56 (t, 2H), 3.5-3.1 (bs, 4H), 3.9 (s, 3H), 2.26 (s, 3H), 1.91 (bs, 4H), 1.7 (bs, 2H). MS (m/z): 475 [MH]⁺, 2Cl.

### EXAMPLE 24

### 3-(4-Chloro-phenyl)-1-(4-methoxy-3-[2-(4-methyl-piperidin-1-yl)-ethoxy]-phenyl)-3,4-dihydro-pyrrol-2-one Hydrochloride Salt

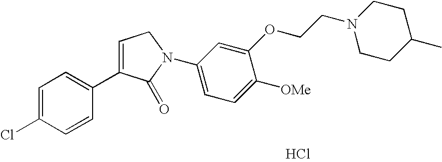

The title compound was prepared in an analogous manner to the one described for the compound of Example 9 and 17 (HCl salt formation).

NMR (¹H, DMSO-d₆): δ 0.9.9+9.76 (2bs, 1H), 8.02 (d, 2H), 7.82 (t, 1H), 7.67 (d, 1H), 7.51 (d, 2H), 7.27 (dd, 1H), 7.06 (d, 1H), 4.61 (d, 2H), 4.36 (t, 2H), 3.78 (s, 3H), 3.59 (d, 2H), 3.48 (m, 2H), 3.04 (q, 2H), 1.81 (m, 1H), 1.60 (bm, 1H), 1.44 (m, 2H), 1.01-0.92 (d-d, 3H). MS (m/z): 441 [MH]⁺, 1Cl.

### EXAMPLE 25

### 3-(4-Chloro-phenyl)-1-(4-methoxy-3-[2-piperidin-1-yl)-ethoxy]-phenyl)-3,4-dihydro-pyrrol-2-one Hydrochloride Salt

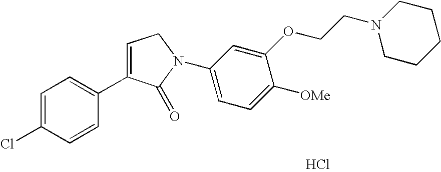

The title compound was prepared in an analogous manner to the one described for the compound of Example 9 and 17 (HCl salt formation).

NMR (¹H, DMSO-d₆): δ 9.8 (bs, 1H), 8.00 (d, 2H), 7.8 (t, 1H), 7.67 (d, 1H), 7.50 (d, 2H), 7.27 (dd, 1H), 7.05 (d, 1H), 4.60 (d, 2H), 4.35 (t, 2H), 3.78 (s, 3H), 3.6/4.4 (m, 4H), 3.00 (m, 2H), 1.85-1.65 (m, 4H), 1.4 (m, 2H). MS (m/z): 427 [MH]⁺, 1Cl

### EXAMPLE 26

### 3-(3,4-Dichloro-phenyl)-1-[4-methoxy-3-(piperidin-3-ylmethoxy)-phenyl]-pyrrolidin-one

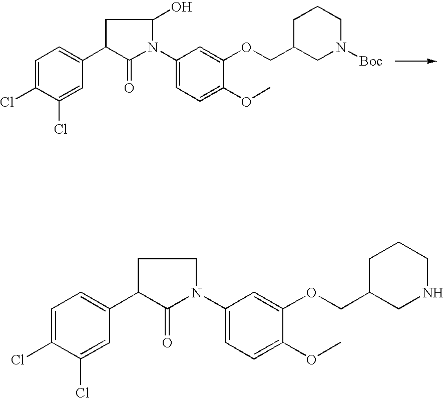

The product was prepared in an analogous manner to Preparation 19.

NMR (¹H, DMSO-d6): δ 7.62-7.60 (m, 2H), 7.47 (t, 1H), 7.33 (dd, 1H), 7.20 (dd, 1H), 6.94 (d, 1H), 3.98 (t, 1H), 3.85 (m, 2H), 3.75 (m, 2H), 3.74 (s, 3H), 3.04 (m, 1H), 2.3 (m, 1H), 2.86 (m, 1H), 2.45 (m, 1H), 2.55 (m, 1H), 2.2 (m, 1H), 1.87 (m, 1H), 1.79 (m, 1H), 1.58 (m, 1H), 1.37 (m, 1H), 1.16 (m, 2H). MS (m/z): 449 [MH]⁺, 2Cl.

### EXAMPLE 27

### 3-(3,4-Dichloro-phenyl)-1-[4-methoxy-3-(1-methyl-piperidin-3-ylmethoxy)-phenyl]-pyrrolidin-one

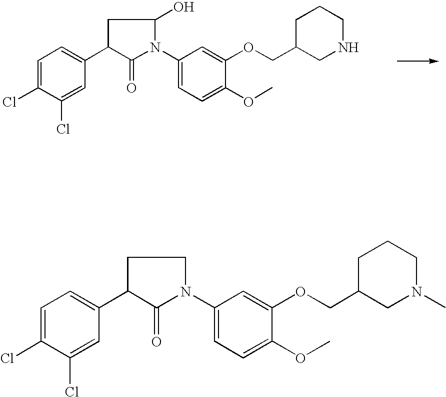

Procedure:

To a solution of 3-(3,4-dichloro-phenyl)-1-[4-methoxy-3-(piperidin-3-ylmethoxy)-phenyl]-pyrrolidin-one (0.064 g, 0.14 mmol) in acetonitrile/dichloromethane 2/1 (3 ml), was added formaldehyde 37% in water (2.5 eq., 0.03 ml) and sodium triacetoxy borohydride (1.5 eq., 45 mg). After 3h the mixture was concentrated, diluited with CH₂Cl₂ and washed with NaHCO₃ 5%. The organic phase was dried over Na₂SO₄ and concentrated in vacuo to give a colorless oil, that was purified by flash chromatography (CH₂Cl₂/MeOH 9/1) to give 52 mg of the title compound (80% yield).

NMR (¹H. DMSO-d6): δ 7.62 (d, 1H), 7.61 (d, 1H), 7.47 (t, 1H), 7.33 (dd, 1H), 7.03 (dd, 1H), 6.96 (d, 1H), 3.99 (t, 1H), 3.86 (dd, 2H), 3.77 (m, 2t), 3.74 (s, 3H), 2.85-2.75 (bs, 1H), 2.7-2.6 (bs, 1H), 2.55 (m, 1H), 2.21 (m, 1H), 2.16 (bs, 3H). 2.0 (bs, 1H), 2.0-1.8 (bs, 2H), 1.69 (m, 1H), 1.62 (m, 1H), 1.6 (m, 1H), 1.48 (m, 1H). MS (m/z): 463 [MH]⁺, 2Cl.

### EXAMPLE 28

### N-{4-[3-(3,4-dichlorophenyl)-2-oxo-2,5-dihydro-1H-pyrrol-1-yl]-2-[2-(1-piperidinyl)ethoxy]phenyl}acetamide Hydrochloride

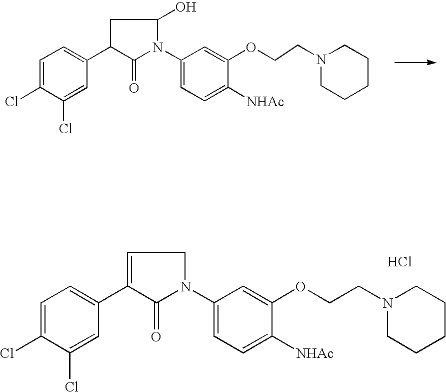

The product was prepared in an analogous manner to Example 9.

NMR (¹H, DMSO-d6): δ 10.2 (bs, 1H), 9.53 (s, 1H), 8.34 (d, 1H), 8.0 (dd, 1H), 8.6(t, 1H), 7.87 (d, 1H) 7.79 (d, 1H), 7.72 (d, 1H), 7.24 (dd, 1H), 4.66 (d, 2H), 4.39 (t, 2H), 3.52 (t, 2H), 3.52 (m, 2H), 2.99 (m, 2H), 2.15 (s, 3H), 1.81 (m, 4H), 1.72 (m, 1H), 1.42 (m, 1H).

MS (m/z): 488 [MH]⁺, 2Cl.

### Example 29

### N-{4-[3-(3,4-dichlorophenyl)-2-oxo-pyrrolidin-1-yl]-2-[2-(1-piperidinyl)ethoxy]phenyl}acetamide Hydrochloride

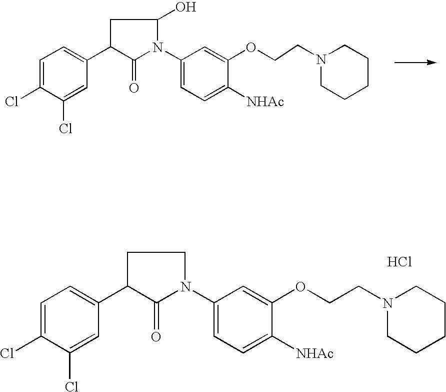

The product was prepared in an analogous manner to Preparation 19.

NMR (¹H, DMSO-d6): δ 10.28 (bs, 1H), 9.55 (s, 1H), 7.89 (d, 1H), 7.63 (d, 1H), 7.63 (m, 2H), 7.34 (dd, 1H), 7.11 (dd, 1H), 4.34 (t, 2H), 4.03 (t, 1H), 3.90 (t, 2H), 3.49 (t, 2H), 3.48 (m, 2H), 2.97 (m, 2H), 2.57 (d, 1H), 2.20 (d, 1H), 2.15 (s, 3H), 1.87 (m, 4H), 1.72 (m, 1H), 1.42 (m, 1H). MS (m/z): 490 [MH]⁺, 2Cl.

### EXAMPLE 30

### 3-(3,4-Dichloro-phenyl)-1-{4-methoxy-3-[2-(4-methyl)-piperidin-1-yl-ethoxy]-phenyl}-3methyl-pyrrolidin-2-one Hydrochloride

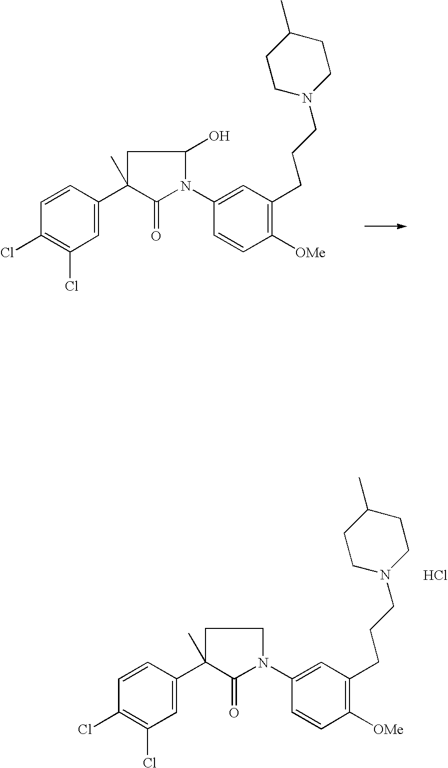

The product was prepared in an analogous manner to Preparation 19

NMR (¹H, DMSO-d6): δ 9.75 (bs, 1H), 7.69 (d, 1H), 7.62 (d, 1H), 7.57 (d, 1H), 7.44 (dd, 1H), 7.15 (dd, 1H), 7.04 (d, 1H), 4.34 (t, 2H), 3.95 (m, 1H), 3.69 (m, 1H), 3.72 (s, 3H), 3.58 (m, 2H), 3.40 (m, 2H), 3.05 (m, 2H), 2.62 (m, 1H), 2.27 (m, 1H), 1.49 (s, 3H), 1.81 (m, 2H), 1.40 (m, 2H), 1.51 (m, 1H), 0.92 (d, 3H). MS (m/z): 491 [MH]⁺, 2Cl.

### EXAMPLE 31

### 3-(3-Chloro-phenyl)-1-[4-methoxy-3-(2-piperidin-1-yl-ethoxy)-phenyl]-pyrrolidin-2-one Hydrochloride Salt

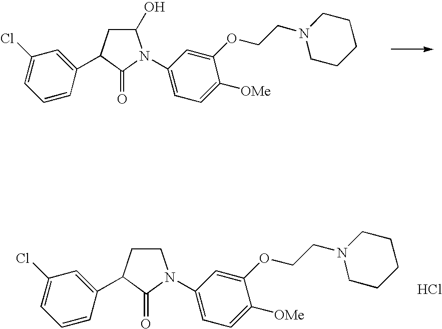

Procedure:

The title compound (52 mg) was obtained as a brown solid in an analogous manner to the one described for Example 21 followed by HCl salt formation as for Example 17.

NMR (¹H, DMSO): δ 9.80 (bs, 1H), 7.57 (d, 1H), 7.42-7.32, 7.28 (m, 4H), 7.16 (dd, 1H), 7.04 (d, 1H), 4.32 (t, 2H), 3.95 (t, 1H), 3.89 (m, 2H), 3.79 (s, 3H), 3.55 (d, 2H), 3.46 (m, 2H), 3.02 (q, 2H), 2.55 (m, 1H), 2.20 (m, 1H), 1.82 (d, 2H), 1.70 (m, 2H), 1.40 (bm, 2H).

MS (m/z, free base): 429 [MH]⁺, 1Cl.

### EXAMPLE 32

### 3-(3-Trifluoromethyl-phenyl)-1-[4-methoxy-3-(2-piperidin-1-yl-ethoxy)-phenyl]-pyrrolidin-2-one

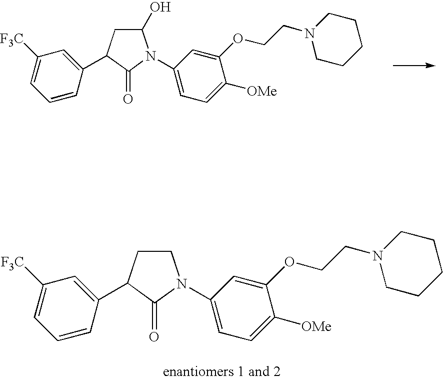

Procedure:

The racemic title compound was obtained in an analogous manner to the one described for Example 21. Separation of the enantiomers was achieved by preparative HPLC using a chiral stationary phase to give Enantiomer 1 (yellow oil, 3 mg) and Enantiomer 2 (colourless gum, 6 mg) of the title compound.

Enantiomer 1: NMR (¹H, CDCl₃): δ 7.56-7.44 (m, 4H), 7.55 (d, 1H), 6.94 (dd, 1H), 6.84 (d, 1 H), 4.17 (t, 2H), 3.88 (m, 3H), 3.83 (s, 3H), 2.84 (m, 2H), 2.66 (m, 1H), 2.54 (bs, 4H), 2.30 (m, 1H), 1.61 (m, 4H), 1.43 (m, 2H).

MS (m/z): 463 [MH]⁺.

Enantiomer 2: NMR (¹H, CDCl₃); MS: Essentially identical to the data for Enantiomer 1.

### EXAMPLE 33

### 3-(3-Trifluoromethyl-phenyl)-1-[4-methoxy-3-(2-piperidin-1-yl-ethoxy)-phenyl]-1,5-dihydro-pyrrol-2-one

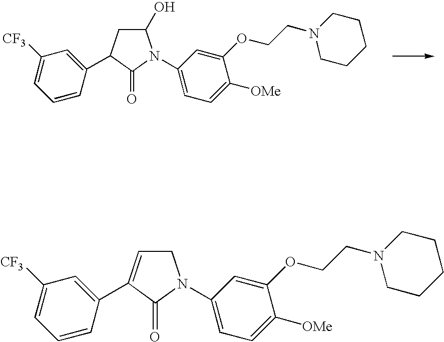

Procedure:

The title compound (20 mg) was obtained as a yellow oil in an analogous manner to the one described for Example 11.

NMR (¹H, DMSO): δ 8.15 (bs, 1H), 8.13 (d, 1H), 7.66 (d, 1H), 7.63 (d, 1H), 7.56 (t, 1H), 7.38 (t, 1H), 7.08 (dd, 1H), 6.90 (d, 1H), 4.49 (d, 2H), 4.23 (t, 2H), 3.88 (s, 3H), 2.87 (t, 2H), 2.55 (bs, 4H), 1.65 (bm, 4H), 1.46 (bs, 2H). MS (m/z): 461 [MH]⁺.

### EXAMPLE 34

### 3-(3-Chloro-phenyl)-5-methoxy-1-[4-methoxy-3-(2-piperidin-1-yl-ethoxy)-phenyl]-pyrrolidin-2-one

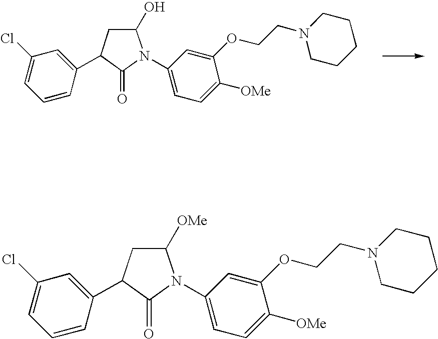

Procedure:

3-(3-Chloro-phenyl)-5-hydroxy-1-[4-methoxy-3-(2-piperidin-1-yl-ethoxy) phenyl]-pyrrolidin-2-one (0.10 g) was treated with MeOH (dry, 4.5 ml) containing pyridinium 4-toluenesulfonate (68 mg) at reflux for 5 h. The mixture was partitioned between aqueous NaHCO₃ and EtOAc. The organic layer was collected, concentrated and submitted to column chromatography (silica gel, CH₂Cl₂/MeOH /NH₃) to give the title compound (3:2 mixture of diastereoisomers, 92 mg) as a brown oil.

NMR (¹H, DMSO): δ 7.5-6.95 (m, 7H), 5.48 (m, 1H), 4.12 (m, 0.6H), 4.02 (t, 2H), 3.90 (m, 0.4H), 3.75 (s, 3H), 3.27 (s, 1.8H), 3.24 (s, 1.2H), 2.85 (m, 0.4H), 2.64 (m, 2H), 2.56 (m, 0.6H), 2.42 (m, 4H), 2.35 (m, 0.6H), 2.05 (m, 0.4H), 1.48 (m, 4H), 1.36 (m, 2H). MS (m/z): 459 [MH]⁺, 1Cl.

### EXAMPLE 35

### 3-(3-Chloro-phenyl)-1-[4-methoxy-3-(2-piperidin-1-yl-ethoxy)-phenyl]-1,5-dihydro-pyrrol-2-one Hydrochloride

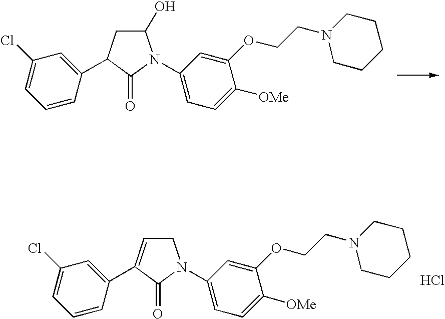

Procedure:

The title compound (52 mg) was obtained as a brown gum in an analogous manner to the one described for Example 9 followed by HCl salt formation as for Example 17.

NMR (¹H, DMSO): δ 9.80 (bs, 1H), 8.11 (t, 1H), 7.94 (dt, 2H), 7.70 (t, 1H), 7.40-7.50 (m, 2H), 7.28 (dd, 1H), 7.07 (d, 1H), 4.63 (d, 2H), 4.35 (t, 2H), 3.79 (s, 3H), 3.57 (d, 2H), 3.50 (m, 2H), 3.03 (q, 2H), 1.85 (d, 2H), 1.71 (m, 2H), 1.20-1.40 (m, 1H).

MS (m/z; free base): 427 [MH]⁺, 1Cl.

### EXAMPLE 36

### 3-(4-Fluoro-phenyl)-1-[4-methoxy-3-(2-piperidin-1-yl-ethoxy)-phenyl]-1,5-dihydro-pyrrol-2-one Hydrochloride

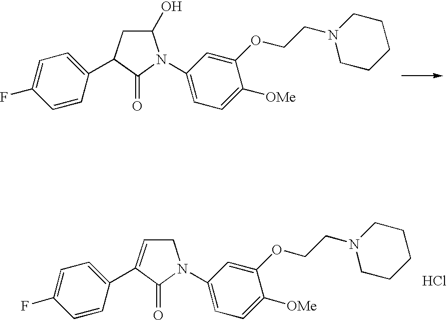

Procedure:

The title compound (28 mg) was obtained as a yellow solid in an analogous manner to the one described for Example 9 followed by HCl salt formation as for Example 17.

NMR (¹H, DMSO): δ 7.96 (m, 2H), 7.55 (t, 1H), 7.23 (d, 1H), 7.21 (dd, 1H), 7.16 (m, 2H), 7.10 (d, 1H), 4.57 (d, 2H), 4.41 (t, 2H), 3.91 (s, 3H), 3.58 (t, 2H), 3.74/3.11 (d/t, 2/2H), 1.99/1.85 (d/t, 2/2H), 1.85/1.60 (m/m, 1/1 H). MS (m/z; free base): 411 [MH]⁺. Mp=207-209° C.

### EXAMPLE 37

### 3-(4-Fluoro-phenyl)-1-[4-methoxy-3(2-piperidin-1-yl-ethoxy)-phenyl]-pyrrolidin-2-one Hydrochloride Salt

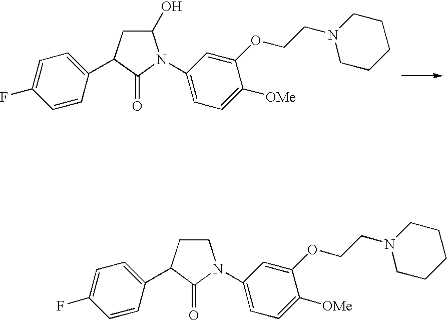

Procedure:

The title compound (52 mg) was obtained as a yellow gum in an analogous manner to the one described for Example 21 followed by HCl salt formation as for Example 17.

NMR (¹H, DMSO-d6): δ 9.8 (s, 1H), 7.59 (d, 1H), 7.34 (m, 2H), 7.17 (m, 2H), 7.16 (dd, 1H), 7.04 (d, 1H), 4.32 (t, 2H), 3.93 (t, 1H), 3.80 (s, 3H), 3.77 (m, 2H), 3.56 (m, 2H), 3.47 (q, 2H), 3.03 (m, 2H), 2.55 (m, 1H), 2.15 (m, 1H), 1.70-1.80 (m, 4H), 1.45 (m, 2H). MS (m/z; free base): 413 [MH]⁺.

### EXAMPLE 38

### 1-{4-Methoxy-3-[2-(4-methyl-piperidin-1-yl)ethoxy]-phenyl}-3-(4-methyl-phenyl)-1,5-dihydro-pyrrol-2-one Hydrochloride Salt

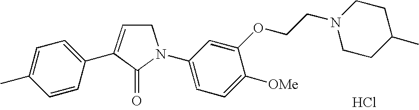

5-Hydroxy-1-{4-methoxy-3-[2-(4-methyl-piperidin-1-yl)-ethoxy]-phenyl}-3-(4-methyl-phenyl)-pyrrolidin-2-one (80 mg, prepared in analogy to Preparations 13, 14 and 46) was dissolved in TFA (2 ml). The reaction mixture was stirred at room temperature overnight, then concentrated in vacuo. A saturated solution of NaHCO₃ was added and the mixture was extracted with ethyl acetate, dried over Na₂SO₄ and concentrated in vacuo. The residue was purified by flash chromathography over silica gel (CH₂Cl₂/MeOH from 1/0 to 98/2) to give 35 mg of the free base of the title compound. The product dissolved in CH₂Cl₂ (1.5ml), at 0° C., was treated with HCl (1 M in Et₂O, 0.17ml) followed by evaporation in vacuo and trituration of the residue with Et₂O to give the title compound (30 mg) as a white solid (40%).

NMR (¹H, CDCl₃): δ 12.4 (bs, 1H), 7.79 (d, 2H), 7.63 (d, 1H), 7.24 (dd, 1H), 7.23 (d, 2H), 7.27 (s, 1H), 6.91 (d, 1H), 4.60 (t, 2H), 4.45 (d, 2H), 3.85 (s, 3H), 3.70 (m, 2H), 3.42 (t, 2H), 2.90 (m, 2H), 2.38 (s, 3H), 2.10/1.80 (m, 4H), 1.6 (m, 1), 1.05 (d, 3H); MS (m/z): 421 [M+H]⁺.

### Examples 39-42 were prepared in analogy to Example 38

### EXAMPLE 39

### 1-{4-Methoxy-3-[2-(piperidin-1-yl)-ethoxy]-phenyl}-3-(4methyl-phenyl)-1,5-dihydro-pyrrol-2-one Hydrochloride Salt

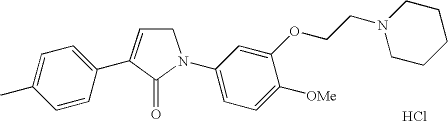

NMR (¹H, DMSO-d₆): δ 10.00 (bs, 1H), 7.87 (d, 2H), 7.68 (s, 1H), 7.67 (s, 1H), 7.28 (dd, 1H), 7.22 (d, 2H), 7.06 (d, 1H), 4.57 (s, 2H), 4.38 (t, 2H), 3.78 (s, 3H), 3.57/3.04 (d/t, 2H/2H), 3.48 (q, 2H), 2.32 (s, 3H), 1.80/1.70 (m, 2H/2H), 1.70/1.40 (m, 1H/1H); MS (m/z): 407 [M+H]⁺.

### EXAMPLE 40

### 3-(4-Bromo-phenyl)-1-{4-methoxy-3-[2-(4-methyl-piperidin-1-yl)-ethoxy]-phenyl}-1,5-dihydro-pyrrol-2-one Hydrochloride Salt

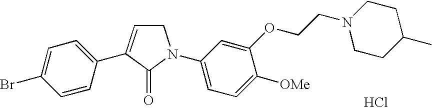

NMR (¹H, DMSO-d₆): δ 10.06 (bs, 1H), 7.95 (d, 2H), 7.83 (t, 1H), 7.67 (d, 1H), 7.63 (d, 2H), 7,28 (dd, 1H), 7.06 (d, 1H), 4.60 (d, 2H), 4.38 (t, 2H), 3.78 (s, 3H), 3.58 (m, 2H), 3.46 (m, 2H), 3.03 (m, 2H), 1.80 (m, 2H), 1.60 (m, 1H), 1.46 (m, 2H), 0.92 (d, 3H); MS (m/z): 485 [M+H]⁺, 1Br.

### EXAMPLE 41

### 1-{4-Methoxy-3-[2-(4-methyl-piperidin-1-yl)ethoxy]-phenyl}-3-(4-trifluoromethyl-phenyl)-1,5-dihydro-pyrrol-2-one Hydrochloride Salt

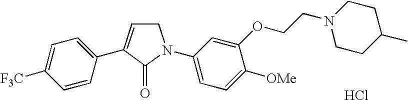

NMR (¹H, DMSO-d₆): δ 10.00/9.83 (bs, 1H), 8.20 (d, 2H), 7.96 (t, 1H), 7.81 (d, 2H), 7.68 (d, 1H), 7,29 (dd, 1H), 7.07 (d, 1H), 4.65 (d, 2H), 4.37 (t, 2H), 3.78 (s, 3H), 3.59 (m, 2H), 3.49 (m, 2H), 3.04 (m, 2H), 1.80/1.40 (m, 4H), 1.60 (m, 1H), 0.91 (d, 3H); MS (m/z): 475 [M+H]⁺.

### EXAMPLE 42

### 3-(2-Chloro-phenyl)-1-{4-methoxy-3-[2-(4-methyl-piperidin-1-yl)-ethoxy]-phenyl}-1,5-dihydro-pyrrol-2-one Hydrochloride Salt

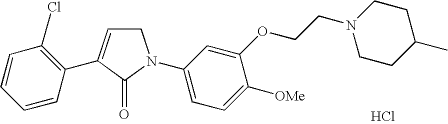

NMR (¹H, DMSO-d₆): δ 9.90 (bs, 1H), 7.61 (d, 1H), 7.56 (t, 1H), 7.50/7.36 (m/m 2H/2H), 7.26 (dd, 1H), 7.04 (d, 1H), 4.64 (d, 2H), 3.75 (s, 3H), 3.34 (t, 2H), 3.56/3.01 (m/m, 2H/2H), 3.44 (m, 2H), 1.78/1.42 (m/m, 2H/2H), 1.55 (bm, 1H), 0.88 (d, 3H); MS (m/z): 441 [M+H]⁺, 1Cl.

### EXAMPLE 43

### 3-(3,4-Dichloro-phenyl)-3-hydroxy-1-{4-methoxy-3-[2-(4-methyl-piperidin-1-yl)-ethoxy]-phenyl}-pyrrolidin-2-one

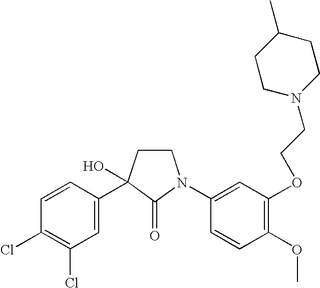

3-(3,4-Dichloro-phenyl)-1-{4-methoxy-3-[2-(4methyl-piperidin-1-yl)-ethoxy]-phenyl}-pyrrolidin-2-one (730 mg, 1.53 mmol) was dissolved in DMF (10 ml), tBuOK (340 mg, 2 eq) was added and the mixture was stirred at room temperature in the presence of air for 15 min. A saturated solution of NaHCO₃ was added and the mixture extracted with CH₂Cl₂. The extracts were dried over Na₂SO₄ and concentrated in vacuo. The residue was purified by column chromathography (silica gel, CH₂Cl₂/MeOH 9/1), to give 260 mg of the title product (34%).

NMR (¹H, CDCl₃): δ 7.55 (m, 2H), 7.40 (d, 1H), 7.25 (d, 1H), 7.00 (d, 1H), 6.85 (d, 1H), 4.15 (m, 2H), 3.80 (s, 3H), 3.70 (m, 1H), 3.00 (m, 2H), 3.85 (m, 2H), 2.50 (m, 1H), 2.40 (m, 1H), 2.10 (m, 1H), 1.90-1.50 (m, 6H), 1.25 (m, 2H), 0.85 (d, 3H). MS (m/z): 493 [MH]⁺ (2Cl).

### EXAMPLE 44

### 3-(3,4-Dichloro-phenyl)-3-fluoro-1-{4-methoxy-3-[2-(4-methyl-piperidin-1-yl)-ethoxy]-phenyl}-pyrrolidin-2-one Hydrochloride Salt

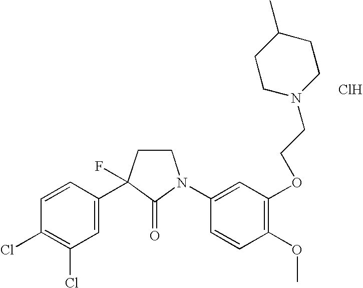

3-(3,4-Dichloro-phenyl)-3-hydroxy-1-{4-methoxy-3-[2-(4-methyl-piperidin-1-yl)ethoxy]-phenyl}-pyrrolidin-2-one (210 mg, 0.43 mmol) was dissolved in CH₂Cl₂ (3 ml), (diethylamino)sulfur trifluoride (DAST, 56 μl, 1 eq) was added at 0° C. and the mixture was stirred for 1 h. A saturated solution of NaHCO₃ was added and the mixture extracted with CH₂Cl₂. The extracts were dried over Na₂SO₄ and concentrated in vacuo. The residue was purified by preparative HPLC to give, after conversion to the hydrochloride salt by treatment with excess HCl (1 M in Et₂O) followed by evaporation in vacuo and trituration of the residue with Et₂O, 72 mg of the title product (34%).

NMR (¹H, Acetone-d₆): δ 13.2 (bs, 1H), 7.81 (d, 1H), 7.69 (m, 2H), 7.61 (dd, 1H), 7.37 (bdd, 1H), 7.09 (d, 1H), 4.66 (bm, 2H), 4.15 (m, 2H), 3.89 (s, 3H), 3.64 (bm, 2H), 3.45 (bm, 2H), 3.09 (bm, 2H), 3.05-2.09 (m, 2H), 2.00-1.6 (bm, 5H), 1.02 (d, 3H). MS (m/z): 495 [MH]⁺(2Cl).

### EXAMPLE 45

### 3-(3,4-Dichloro-phenyl)-1-{4-methoxy-3-[(1-methyl-pyrrolidin-2-yl)-methoxy]-phenyl}-pyrrolidin-2one

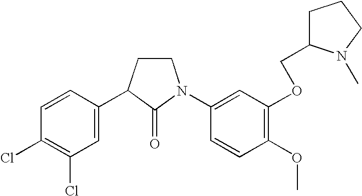

The product was prepared in an analogous manner to Example 27.

NMR (¹H, CD₃OD): δ 7.57 (d, 1H), 7.54 (d, 1H), 7.53 (d, 1H), 7.29 (dd, 1H), 7.12/7.09 (m, 2H), 4.45/4.2 (m, 2H), 3.97 (m, 3H), 3.90 (m, 1H), 3.87 (s, 3H), 3.70/3.25 (m, 2H), 3.14 (s, 3H), 2.65/2.30 (m, 2H), 2.4/2.1 (m, 2H), 2.25/2.1 (m, 2H). MS (m/z): 449 [MH]⁺ (2Cl).

### EXAMPLE 46

### 3-(3,4-Dichloro-phenyl)-1-{4-methoxy-3-[(1-methyl-pyrrolidin-2-yl)-methoxy]-phenyl}-3,4-dihydro-pyrrol-2-one Hydrochloride Salt

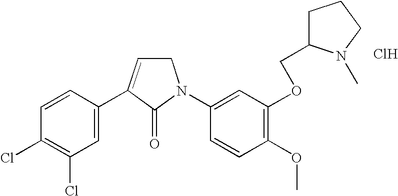

3-(3,4-Dichloro-phenyl)-1-{4-methoxy-3-[(1-methyl-pyrrolidin-2-yl)-methoxy]-phenyl}-pyrrolidin-2-one (134 mg, 0.276 mmol) was dissolved in DMF (1 ml), tBuOK (124 mg, 4 eq) was added and the mixture was stirred at room temperature in the presence of air for 10 min. A saturated solution of NaHCO₃ was added and the mixture extracted with CH₂Cl₂. The extracts were dried over Na₂SO₄ and concentrated in vacuo. The residue was suspended in HCl 37% and heated in a microwave oven to 130° C. for 10 minutes. The solvent was removed and the residue was purified by column chromathography (silica gel, CH₂Cl₂/MeOH 9/1), to give, after conversion to the hydrochloride salt by treatment with excess HCl (1 M in Et₂O) followed by evaporation in vacuo and trituraton of the residue with Et₂O, 10 mg of the title product (8%).

NMR (¹H, CDCl₃): δ 12.65 (bs, 1H), 8.1 (s, 1H), 7.75 (d, 1H), 7.70 (s, 1H), 7.50 (d, 1H), 7.4 (d, 1H), 7.35 (s, 1H), 6.90 (d, 1H), 4.85 (t, 1H), 4.55 (d, 1H), 4.35 (d, 1H), 3.95 (s, 1H), 3.85 (s, 3H), 3.65 (bs, 1H), 3.10 (s, 3H), 2.90 (s, 2H), 2.30 (bm, 2H), 2.10 (bm, 2H). MS (m/z): 447 [MH]⁺ (2Cl).

1. A compound of formula (I) or a pharmaceutically acceptable salt 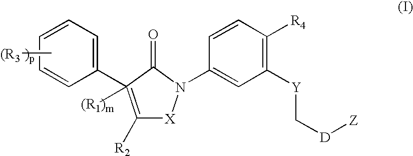
thereof:
wherein:
R₁ is hydrogen, hydroxy, fluoro, chloro, C₁₋₆alkyl, C₃₋₇cycloalkyl, C₃₋₇cycloalkyloxy, C₁₋₆alkoxy or haloC₁₋₆alkoxy;
m is 0 when 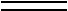
is a double bond and m is 1 when 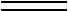
is a single bond;
R₂ is hydrogen, halogen, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy, C₁₋₆alkylthio, amino, mono- or di-C₁₋₆alkylamino or an N-linked 4 to 7 membered heterocyclic group;
X is —(CH₂—CH₂)—, —(CH═CH)—, —(CH₂)₃—, —C(CH₃)₂—, —(CH═CH—CH₂)—, —(CH₂—CH═CH)— or a group —(CHR₅)— wherein R₅ is hydrogen, halogen, hydroxy, cyano, nitro, C₁₋₆alkyl, C₃₋₇cycloalkyl, C₃₋₇cycloalkyloxy, haloC₁₋₆alkyl, C₁₋₆alkoxy, haloC₁₋₆alkoxy or C₁₋₆alkylthio;
R₃ is halogen, cyano, C₁₋₆alkyl, C₃₋₇cycloalkyl, C₃₋₇cycloalkyloxy, C₁₋₆alkoxy, C₁₋₆alkylthio, hydroxy, amino, mono- or di-C₁₋₆alkylamino, an N-linked 4 to 7 membered heterocyclic group, nitro, haloC₁₋₆alkyl, haloC₁₋₆alkoxy, aryl, arylC₁₋₆alkyl, arylC₁₋₆alkyloxy, arylC₁₋₆alkylthio or COOR₆, CONR₇R₈ or COR₉ wherein R₆, R₇, R₈ and R₉ are independently hydrogen or C₁₋₆alkyl;
p is 0, 1 or 2 or 3;
R₄ is hydrogen, halogen, hydroxy, cyano, nitro, C₁₋₆alkyl, C₁₋₆alkanoyl, C₃₋₇cycloalkyl, C₃₋₇cycloalkyloxy, haloC₁₋₆alkyl, C₁₋₆alkoxy, haloC₁₋₆alkoxy,
C₁₋₆alkylthio, amino, mono- or di-C₁₋₆alkylamino or an N-linked 4 to 7 membered heterocyclic group;
Y is oxygen, sulfur, —CH₂— or NR₁₀ wherein R₁₀ is hydrogen or C₁₋₆alkyl;
D is a single bond, —CH₂—, —(CH₂)₂— or —CH═CH—; and
Z is an optionally substituted C-linked 4 to 7 membered heterocyclic group containing at least one nitrogen, an optionally substituted N-linked 4 to 7 membered heterocyclic group, or Z is —NR₁₁R₁₂ where R₁₁ and R₁₂ are independently hydrogen or C₁₋₆alkyl.

2. A compound as claimed in claim 1, wherein X is —CH₂—.

3. A compound as claimed in claim 1, wherein when 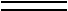
is a single bond, R₁ is hydrogen, hydroxy or C₁₋₆alkoxy.

4. A compound as claimed in claim 1 having the following formula (Ia): 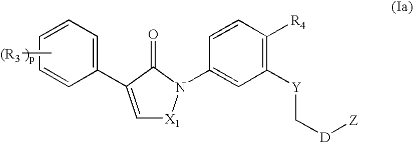
wherein R₃, p, R₄, Y, D, Z, 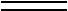
are as defined in claim 1 and X₁ is —CH₂— or —HC(OH)—.

5. A compound as claimed in claim 1, wherein p is 1 or 2 and R₃ is/are halogen, particularly chloro or fluoro, attached at the 3 or the 3,4-positions of the phenyl ring.

6. A compound as claimed in claim 1, wherein R₄ is C₁₋₆alkoxy (particularly methoxy), OCF₃, halogen or cyano.

7. A compound as claimed in claim 1 wherein D is —CH₂—.

8. A compound as claimed in claim 1, wherein Y is oxygen.

9. A compound as claimed in claim 1, wherein Z is an optionally substituted N-linked 4 to 7 membered heterocyclic group.

10. A compound as claimed in claim 9, wherein Z is piperidyl.

11. A compound as claimed in claim 1 which is:
3-(3,4-Dichloro-phenyl)-3-hydroxy-1-[4-methoxy-3-(2-piperidin-1-yl-ethoxy)-phenyl]-pyrrolidin-2-one;
3-(3,4-Dichloro-phenyl)-3-hydroxy-1-[4-methoxy-3-(2-morpholin-4-yl-ethoxy)-phenyl]-pyrrolidin-2-one;
3-(3,4-Dichloro-phenyl)-1-[4-methoxy-3-(2-piperidin-1-yl-ethoxy)-phenyl]-pyrrolidin-2-one;
1-[4-Chloro-3-(2-piperidin-1-yl-ethoxy)-phenyl]-3-(3,4-dichloro-phenyl)-pyrrolidin-2-one;
1-[4-Chloro-3-(2-piperidin-1-yl-ethoxy)-phenyl]-3-(3,4-dichloro-phenyl)-3-hydroxy-pyrrolidin-2-one;
3-(3,4-Dichloro-phenyl)-1-(4-methoxy-3-[2-(4-methyl-piperidin-1-yl)-ethoxy]-phenyl)-pyrrolidin-2-one;
3-(3,4-Dichloro-phenyl)-1-[4-methoxy-3-(2-piperidin-1-yl-ethoxy)-phenyl]-1,5-dihydro-pyrrol-2-one;
3-(3,4-Dichloro-phenyl)-1-[4-methoxy-3-(2-morpholin-4-yl-ethoxy)-phenyl]-1,5-dihydro-pyrrol-2-one;
1-[4-Chloro-3-(2-piperidin-1-yl-ethoxy)-phenyl]-3-(3,4-dichloro-phenyl)-1,5-dihydro-pyrrol-2-one;
3-(3,4-Dichloro-phenyl)-1-[4-methoxy-3-(2-pyrrolidin-1-yl-ethoxy)-phenyl]-1,5-dihydro-pyrrol-2-one;
3-(3-Fluoro-phenyl)-1-[4-methoxy-3-(2-piperidin-1-yl-ethoxy)-phenyl]-1,5-dihydro-pyrrol-2-one;
3-(3,4-Dichloro-phenyl)-1-(-3-[2-(4,4-difluoro-piperidin-1-yl)-ethoxy]-4-methoxy-phenyl)-1,5-dihydro-pyrrol-2-one;
3-(3-Fluoro-phenyl)-5-hydroxy-1-[4-methoxy-3-(2-piperidin-1-yl-ethoxy)-phenyl]-pyrrolidin-2-one;
1-[4-Chloro-3-(2-piperidin-1-yl-ethoxy)-phenyl]-3-(3,4-dichloro-phenyl)-5-hydroxy-pyrrolidin-2-one;
3-(3,4-Dichloro-phenyl)-1-(-3-[2-(4,4-difluoro-piperidin-1-yl)-ethoxy]-4-methoxy-phenyl)-5-hydroxy-pyrrolidin-2-one;
3-(3-Fluoro-phenyl)-1-[4-methoxy-3-(2-piperidin-1-yl-ethoxy)-phenyl]-pyrrolidin-2-one;
3-(3,4-Dichloro-phenyl)-1-[4-methoxy-3-(2-pyrrolidin-1-yl-ethoxy)-phenyl]-pyrrolidin-2-one;
3-(3,4-Dichloro-phenyl)-1-[4-methoxy-3-(2-piperidin-1-yl-ethoxy)-phenyl]-3-methyl-pyrrolidin-2-one hydrochloride salt;
3-(3-Chloro-phenyl)-1-{4-methoxy-3-[2-(4-methyl-piperidin-1-yl)-ethoxy]-phenyl}-1,5-dihydro-pyrrol-2-one hydrochloride salt;
3-(3,4-Dichloro-phenyl)-1-[4-methoxy-3-(2-piperidin-1-yl-ethoxy)-phenyl]-3,4-dihydro-1 H-pyridin-2-one;
3-(3,4-Dichloro-phenyl)-1-[4-methoxy-3-(2-piperidin-1-yl-ethoxy)-phenyl]-piperidin-2-one;
3-(3,4-Dichloro-phenyl)-1-(4-methoxy-3-[2-(4-methyl-piperidin-1-yl)-ethoxy]-phenyl)-3,4-dihydro-pyrrol-2-one;
3-(3,4-Dichloro-phenyl)-1-(4-methoxy-3-[2-(4-methyl-piperidin-1-yl)-ethoxy]-phenyl)-4-methyl-1,5-dihydro-pyrrol-2-one;
3-(4-Chloro-phenyl)-1-(4-methoxy-3-[2-(4-methyl-piperidin-1-yl)-ethoxy]-phenyl)-3,4-dihydro-pyrrol-2-one;
3-(4-Chloro-phenyl)-1-(4-methoxy-3-[2-piperidin-1-yl)-ethoxy]-phenyl)-3,4-dihydro-pyrrol-2-one;
3-(3,4-Dichloro-phenyl)-1-[4-methoxy-3-(piperidin-3-ylmethoxy)-phenyl]-pyrrolidin-one;
3-(3,4-Dichloro-phenyl)-1-[4-methoxy-3-(1-methyl-piperidin-3-ylmethoxy)-phenyl]-pyrrolidin-one;
N-{4-[3-(3,4-dichlorophenyl)-2-oxo-2,5-dihydro-1H-pyrrol-1-yl]-2-[2-(1-piperidinyl)ethoxy]phenyl}acetamide;
N-{4-[3-(3,4-dichlorophenyl)-2-oxo-pyrrolidin-1-yl]-2-[2-(1-piperidinyl)ethoxy]phenyl}acetamide;
3-(3,4-Dichloro-phenyl)-1-{4-methoxy-3-[2-(4-methyl)-piperidin-1-yl-ethoxy]-phenyl}-3-methyl-pyrrolidin-2-one;
3-(3-Chloro-phenyl)-1-[4-methoxy-3-(2-piperidin-1-yl-ethoxy)-phenyl]-pyrrolidin-2-one;
3-(3-Trifluoromethyl-phenyl)-1-[4-methoxy-3-(2-piperidin-1-yl-ethoxy)-phenyl]-pyrrolidin-2-one;
3-(3-Trifluoromethyl-phenyl)-1-[4-methoxy-3-(2-piperidin-1-yl-ethoxy)-phenyl]-1,5-dihydro-pyrrol-2-one;
3-(3-Chloro-phenyl)-5-methoxy-1-[4-methoxy-3-(2-piperidin-1-yl-ethoxy)-phenyl]-pyrrolidin-2-one;
3-(3-Chloro-phenyl)-1-[4-methoxy-3-(2-piperidin-1-yl-ethoxy)-phenyl]-1,5-dihydro-pyrrol-2-one;
3-(4-Fluoro-phenyl)-1-[4-methoxy-3-(2-piperidin-1-yl-ethoxy)-phenyl]-1,5-dihydro-pyrrol-2-one;
3-(4-Fluoro-phenyl)-1-[4-methoxy-3-(2-piperidin-1-yl-ethoxy)-phenyl]-pyrrolidin-2-one;
1-{4-Methoxy-3-[2-(4-methyl-piperidin-1-yl)-ethoxy]-phenyl}-3-(4-methyl-phenyl)-1,5-dihydro-pyrrol-2-one;
1-{4-Methoxy-3-[2-(piperidin-1-yl)-ethoxy]-phenyl}-3-(4-methyl-phenyl)-1,5-dihydro-pyrrol-2-one;
3-(4-Bromo-phenyl)-1-{4-methoxy-3-[2-(4-methyl-piperidin-1-yl)-ethoxy]-phenyl}-1,5-dihydro-pyrrol-2-one;
1-{4-Methoxy-3-[2-(4-methyl-piperidin-1-yl)-ethoxy]-phenyl}-3-(4-trifluoromethyl-phenyl)-1,5-dihydro-pyrrol-2-one;
3-(2-Chloro-phenyl)-1-{4-methoxy-3-[2-(4-methyl-piperidin-1-yl)-ethoxy]-phenyl}-1,5-dihydro-pyrrol-2-one;
3-(3,4-Dichloro-phenyl)-3-hydroxy-1-{4-methoxy-3-[2-(4-methyl-piperidin-1-yl)-ethoxy]-phenyl}-pyrrolidin-2-one;
3-(3,4-Dichloro-phenyl)-3-fluoro-1-{4-methoxy-3-[2-(4-methyl-piperidin-1-yl)-ethoxy]-phenyl}-pyrrolidin-2-one;
3-(3,4-Dichloro-phenyl)-1-{4-methoxy-3-[(1-methyl-pyrrolidin-2-yl)-methoxy]-phenyl}-pyrrolidin-2-one;
3-(3,4-Dichloro-phenyl)-1-{4-methoxy-3-[(1-methyl-pyrrolidin-2-yl)-methoxy]-phenyl}-3,4-dihydro-pyrrol-2-one;
or a pharmaceutically acceptable salt thereof.

12. A process for the preparation of a compound as defined in claim 1, which process comprises:
(a) reacting a compound of formula (II): 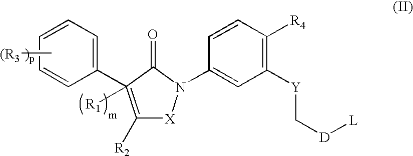
wherein R₁, R₂, R₃, R₄, m, p, X, 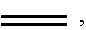
Y and D are as defined for formula (I), and L is a leaving group, with a compound of formula (Ill):
Z-H (III)
wherein Z is as defined for formula (I); or
(b) cyclising a compound of formula (IV): 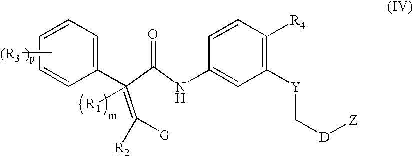
wherein R₁, R₂, m, R₃, p, R4, Y, D, Z and 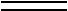
are as defined for formula (I) and G is a group —X═CH₂, wherein X is as defined for formula (I), dehydrogenated as required;
optionally followed by:
removing any protecting groups; and/or
converting a compound of formula (I) into another compound of formula (I); and/or
forming a pharmaceutically acceptable salt.

13. A pharmaceutical composition comprising a compound as defined in claim 1 and a pharmaceutically acceptable carrier or excipient.

14. A process for preparing a pharmaceutical composition as defined in claim 13, the process comprising mixing a compound a compound as defined in claim 1 and a pharmaceutically acceptable carrier or excipient.

15. A compound as defined in claim 1 for use as a therapeutic substance.

16. A compound as defined in claim 1 for use in the treatment of a CNS disorder.

17. A compound as defined in claim 1 for use in the treatment of depression or anxiety.

18. A method of treatment of a CNS disorder in a mammal including a human, which comprises administering to the sufferer a therapeutically safe and effective amount of a compound as defined in claim 1.

19. A method of treatment of depression and or anxiety in a mammal including a human, which comprises administering to the sufferer a therapeutically safe and effective amount of a compound as defined in claim 1.

20. Use of a compound as defined in claim 1 in the manufacture of a medicament for use in the treatment of a CNS disorder.

21. The use of a compound as defined in claim 1 in the manufacture of a medicament for use in the treatment of depression or anxiety.
